(19) United States
(12) Patent Application Publication
(10) Pub. No.: US 2021/0283218 A1
(43) Pub. Date: Sep. 16, 2021

(54) IMMUNOTOLERANCE WITH HEAT SHOCK PROTEINS

(71) Applicant: Serenity Bioworks Inc., Kitchener (CA)

(72) Inventors: Cody SHIRRIFF, Waterloo (CA); Spencer BERG, Waterloo (CA)

(21) Appl. No.: 17/241,310

(22) Filed: Apr. 27, 2021

Related U.S. Application Data
(63) Continuation of application No. PCT/CA2019/051570, filed on Nov. 5, 2019.
(60) Provisional application No. 62/755,955, filed on Nov. 5, 2018.

(51) Int. Cl. A61K 38/17; A61K 35/17; A61K 35/15; A61K 35/28; A61K 31/711; A61K 31/7105; C12N 15/86; A61P 37/02

(52) U.S. Cl. CPC A61K 38/1709; A61K 35/17; A61K 35/15; A61K 35/28; C12N 2750/14143; A61K 31/7105; C12N 15/86; A61P 37/02; A61K 31/711

(57) ABSTRACT

Disclosed herein are compositions comprising at least one heat shock protein or a functional fragment thereof, wherein a therapeutically effective amount of the at least one heat shock protein or the functional fragment thereof induces immunotolerance to a therapeutic agent when the therapeutically effective amount of the composition is administered to a subject. Also, provided herein are methods of inducing immunotolerance in a subject. The methods comprise administering a composition comprising at least one heat shock protein or a functional fragment thereof to the subject, and administering a therapeutic agent, wherein the composition induces immunotolerance to the therapeutic agent in the subject.

### CROSS-REFERENCE TO RELATED APPLICATION

This application is a continuation of International Application No. PCT/CA2019/051570, filed Nov. 5, 2019, which claims priority to U.S. 62/755,955, filed Nov. 5, 2018, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Apr. 15, 2021, is named 55409_701_301_SL.txt and is 127,438 bytes in size.

### BACKGROUND

Many cutting-edge medical treatments, such as gene therapy, enzyme replacement therapy, biologic agent therapy, and cell-based therapy, are plagued with a common side effect of stimulating an unwanted immune response against the administered therapeutic agent. For example, adeno-associated viruses (AAVs), which are one the most effective and well understood vectors for gene therapy, have been shown to induce activation of dendritic cells (DCs), even though AAV is generally thought to be only weakly immunogenic. Even if a vector itself fails to trigger the immune system, it is still possible to later develop immunity against the vector's transgene product. Indeed, every first AAV dose administered to a patient, outside of immune privileged sites, results in increased neutralizing antibodies to AAV; these antibodies may limit the ability of a patient to receive subsequent administrations of the therapy. Similarly, many of the most important and commercially-successful biologics, e.g., insulin, Adalimumab (HUMIRA), and Factor VIII, are immunogenic and generate undesirable immune responses in patients. Increased tolerance to AAV-delivered therapies and to biologics, as examples, could provide patients more effective therapies over the long-term. Accordingly, there remains an unmet need for methods and compositions that help overcome immunogenicity of a therapeutic agent and promote immunotolerance.

### SUMMARY

The present invention addresses this need. Accordingly, the present disclosure provides methods and compositions that overcome immunogenicity of therapeutic agents and promote immunotolerance.

An aspect of the present disclosure is a method for generating an immune tolerizing effect against a therapeutic agent administered to a subject. The method comprises administering to the subject a therapeutic agent and administering to the subject an effective amount of a heat shock protein to generate an immune tolerizing effect against the therapeutic agent.

In embodiments, the therapeutic agent is administered concurrently with the administering of the heat shock protein, the therapeutic agent is administered to the subject before the administering of the heat shock protein, or the therapeutic agent is administered to the subject after the administering of the heat shock protein. In some embodiments, the heat shock protein may be administered multiple times, such as both prior and concurrent with the therapeutic agent, prior and after administration of the therapeutic agent, concurrent and after the administration of the therapeutic agent and before, concurrent with, and after administration with the therapeutic agent.

In embodiments, the immune tolerizing effect comprises an increased amount of antigen-specific regulatory T cells (Tregs) in the subject. The antigen-specific Tregs recognize the therapeutic agent or a portion thereof. In embodiments, the immune tolerizing effect results in an increased amount of an expression level of CD25 and/or FoxP3 on the antigen-specific Tregs.

In embodiments, the administering to the subject a therapeutic agent comprises administering one or more first lower doses of the therapeutic agent and followed by one or more doses of the therapeutic agent at a higher dose. In some embodiments, the one or more first doses are lower in amount than what is understood to be a therapeutically effective amount. In some embodiments, the one or more first doses are administered at a low dosage. In some embodiments, the one or more first doses are administered at a dosage than is less effective than the doses that follow at a higher amount. In embodiments, the one or more higher doses is a therapeutically effective amount. In embodiments, the heat shock protein is administered concurrently with the one or more first low doses of the therapeutic agent; the heat shock protein is administered before the one or more first low doses of the therapeutic agent; the heat shock protein is administered after the one or more first low doses of the therapeutic agent; or the heat shock protein is administered before and following the one or more first low doses of the therapeutic agent. In embodiments, the heat shock protein is also administered before, concurrently with, or after the one or more doses of the therapeutic agent administered at a therapeutically effective amount, if administered.

In embodiments, the immune tolerizing effect comprises a reduction of the amount of an anti-therapeutic-agent antibody in the subject.

In embodiments, the heat shock protein is αB-crystallin (CRYAB), αA-crystallin (CRYAA), HSP60, HSP70, HSP72, HSP84, HSP90, HSP104, GP96, HSP33, HSP27, HSP22, HSP20, HSP12, HSP10, HSP7, or a functional fragment thereof.

In embodiments, the heat shock protein comprises a small heat shock protein (sHsp) or a functional fragment thereof. In embodiments, the sHsp comprises one or more features selected from (i) a subunit molecular mass between about 12 and about 43 kDa, (ii) an α-crystallin domain, (iii) an N-terminal domain, and (iv) C-terminal extension.

In embodiments, the CRYAB comprises a sequence selected from the group consisting of SEQ ID NO: 18-25. In embodiments, the CRYAB comprises a sequence of SEQ ID NO: 18. In embodiments, the CRYAB comprises a sequence that is at least 65%, 70%, 75%, 80%, 85%, 90%, 95%, 96%, 97%, 98%, 99% identical to SEQ ID NO:18. In embodiments, the functional fragment of CRYAB is selected from the group consisting of SEQ ID NO: 46 to SEQ ID NO: 48. In embodiments, the functional fragment of CRYAB is at least 5 amino acids in length, e.g., at least 5, 6, 7, 8, 9, 10, 11, 12, 13, 14, 15, 16, 17, 18, 19, 20, 21, 25, 30, 40, 50, 60, 70, 80, 90, 100, or 125, and any number amino of acids therebetween.

In embodiments, the therapeutic agent comprises a nucleic acid, a peptide, a protein, a compound, a chemotherapeutic, a cell, or any combination thereof.

In embodiments, the therapeutic agent comprising a nucleic acid that is DNA (e.g., plasmid DNA and linear DNA) or that is RNA (e.g., mRNA, antisense RNA, miRNA, siRNA, or gRNA). In embodiments, the nucleic acid comprises a viral vector.

In embodiments, the therapeutic agent is selected from the group consisting of a biologic, an antibody, and an antigen binding fragment. In embodiments, the therapeutic agent is administered as a nucleic acid encoding a peptide/protein-based therapeutic agent, a biologic, an antibody, or an antigen binding fragment; the nucleic acid is transcribed and/or translated by a cell. In embodiments, the nucleic acid is DNA (e.g., plasmid DNA and linear DNA) or is RNA (e.g., mRNA, antisense RNA, miRNA, and siRNA). In embodiments, the nucleic acid is packaged in a viral vector.

In embodiments, the therapeutic agent includes a packaging component.

In embodiments, the packaging component is a viral vector, a virus, or a virus-like particle. The viral vector may be a lentivirus; the viral vector may be an adeno-associated virus (AAV). Examples of AAV include AAV1, AAV2, AAV4, AAV5, AAV6, AAV7, AAV8, AAV9, AAV2/5, AAV2/2, AAV-DJ, and AAV-DJ8, and any combination thereof. In embodiments, the AAV is AAV1, AAV5, AAV6, AAV8, or AAV9. Also, envisioned are AAVs that have been modified to increase cell specificity and/or avoid preexisting immunity to the AAV capsid. See, e.g., Büning & Srivastava (2019), “Capsid modifications for targeting and improving the efficacy of AAV vectors.” Molecular Therapy—Methods & Clinical Development; 12:248-265; Smith and Agbandje-McKenna (2018), “Creating an arsenal of Adeno-associated virus (AAV) gene delivery stealth vehicles.” PLoS Pathog, 14(5): e 1006929; Yang et al., (2011), “Directed evolution of adeno-associated virus (AAV) as vector for muscle gene therapy.” Methods Mol Biol. 2011; 709:127-39; and Schaffer & Maheshri (2004), “Directed evolution of AAV mutants for enhanced gene delivery.” Conf Proc IEEE Eng Med Biol Soc. 2004; 5:3520-3. The contents of each of which is incorporated by reference in its entirety.

In embodiments, the packaging component is a microcapsule. The microcapsule may be a liposome, an albumin microsphere, a microemulsion, a nanoparticle (e.g., a lipid nanoparticle), and a nanocapsule and/or may comprise hydroxylmethylcellulose, gelatin-microcapsules, and/or polymethylmethacrylate.

In embodiments, the immune tolerizing effect (provided by the heat shock protein) is directed against the packaging component or a portion thereof. In some embodiments, the immune tolerizing effect (provided by the heat shock protein) is directed against a cargo carried by a packaging component or a portion thereof.

In some embodiments, a therapeutic agent does not include a packaging agent, such as a naked protein, peptide, antibody, enzyme, nucleic acid or viral vector; in such embodiments, the immune tolerizing effect (provided by the heat shock protein) is directed against the naked protein, peptide, antibody, enzyme, or nucleic acid or a component or portion thereof.

In embodiments, the subject is a mammal. In embodiments, the subject is a human. In embodiments, the subject is a companion animal or a livestock animal.

In embodiments, the method comprises administering to the subject one or more subsequent doses of the therapeutic agent, e.g., a gene therapy, wherein one or more subsequent doses is effective to generate a therapeutic response. In embodiments, the therapeutic response to the subsequent dose of the therapeutic agent is enhanced or improved as compared with the response when the heat shock protein is not administered; the administration of the heat shock protein reduces, prevents, or alleviates an immune response to the subsequent dose of the therapeutic agent; or the administration of the heat shock protein reduces, prevents, or alleviates inactivation of the subsequent dose of the therapeutic agent.

In embodiments, the immune tolerizing effect comprises, but is not limited to, the modulation of the expression or secretion of one or more anti-inflammatory cytokine or related proteins such as IFNγ, IL-10, TGFβ, IL-35, IL-4, IL-12, PTX3, TSG6/TNFAIP6, and CCL20; or the immune tolerizing effect comprises induction of apoptosis through upregulation of one or more of perforin and granzyme AB pathway, Fas/Fas-L pathway, TRAIL, galectin-1, galectin-9/TIM-3 pathway; or the immune tolerizing effect comprises the upregulation of CTLA-4, PD-1, PD-L1, LAG3, SLAMF1 and/or a change in the number and/or ratio of regulatory T cells, Tr1 cells, regulatory B cells, double negative regulatory T cells and/or exhausted T cells (e.g., low IL-2, low proliferation, and low IFNγ T cells); or the immune tolerizing effect comprises a disruption in the metabolic pathways in T effector cells (e.g. cAMP); or through modulation of antigen-presenting cell (e.g. dendritic cells) maturation and function as a consequence of CTLA4:CD80/86 interaction and upregulation of indoleamine 2,3-dioxygenase (IDO).

In embodiments, the method further comprises administering to the subject an immunosuppressant. In embodiments, immunosuppressant is administered to the subject concurrently with the administering of the heat shock protein, the immunosuppressant is administered to the subject before the administering of the heat shock protein, or the immunosuppressant is administered to the subject after the administering of the heat shock protein. In embodiments, the immunosuppressant is administered to the subject concurrently with the administering of the therapeutic agent, the immunosuppressant is administered to the subject before the administering of the therapeutic agent, or the immunosuppressant is administered to the subject after the administering of the therapeutic agent.

In embodiments, the subject does not have a disease or disorder associated with inflammation. In embodiments, the methods and compositions of the present disclosure are not directed towards reducing inflammation associated with a disease.

Administration methods for use with the compositions and methods disclosed herein can be by any suitable route of administration and include, but are not limited to injection, inhalation, absorption, ingestion, or other methods.

Another aspect of the present disclosure is a pharmaceutical composition for use in any herein disclosed method.

Yet another aspect of the present disclosure is a plurality of pharmaceutical compositions for use in any herein disclosed method.

In another aspect, the present disclosure provides a pharmaceutical composition comprising an immune tolerizing effective amount of a heat shock protein. The immune tolerizing effective amount of the heat shock protein reduces and/or inhibits an immune response to a therapeutic agent when administered to a subject.

In embodiments, the immune tolerizing effective amount of a heat shock protein in a pharmaceutical composition increases an amount of antigen-specific regulatory T cells (Tregs) in the subject. The antigen-specific Tregs recognize the therapeutic agent or a portion thereof and/or the immune tolerizing effective amount of a heat shock protein increases an amount of an expression level of CD25 and/or FoxP3 on the antigen-specific Tregs.

In embodiments, the heat shock protein in a pharmaceutical composition is αB-crystallin (CRYAB), αA-crystallin (CRYAA), HSP60, HSP70, HSP72, HSP84, HSP90, HSP104, GP96, HSP33, HSP27, HSP22, HSP20, HSP12, HSP10, HSP7, or a functional fragment thereof.

In embodiments, the heat shock protein in a pharmaceutical composition comprises a small heat shock protein (sHsp) or a functional fragment thereof. In embodiments, the sHsp comprises one or more features selected from (i) a subunit molecular mass between about 12 and about 43 kDa, (ii) an α-crystallin domain, (iii) an N-terminal domain and (iv) C-terminal extension.

In embodiments, the heat shock protein in a pharmaceutical composition is CRYAB or CRYAA.

In embodiments, the CRYAB in a pharmaceutical composition comprises a sequence selected from the group consisting of SEQ ID NO: 18-25. In embodiments, the CRYAB comprises a sequence of SEQ ID NO: 18. In embodiments, the CRYAB comprises a sequence that is at least 65%, 70%, 75%, 80%, 85%, 90%, 95%, 96%, 97%, 98%, 99% identical to SEQ ID NO:18. In embodiments, the functional fragment of CRYAB is selected from the group consisting of SEQ ID NO: 46 to SEQ ID NO: 48. In embodiments, the functional fragment of CRYAB is at least 5 amino acids in length, e.g., at least 5, 6, 7, 8, 9, 10, 11, 12, 13, 14, 15, 16, 17, 18, 19, 20, 21, 25, 30, 40, 50, 60, 70, 80, 90, 100, or 125, and any number of amino acids therebetween.

In embodiments, the immune tolerizing effect is directed against an active agent and/or against a packaging component.

In embodiments, the composition further comprises a therapeutic agent which comprises a nucleic acid, a peptide, a protein, a compound, a chemotherapeutic, a cell, or any combination thereof. In embodiments, the therapeutic agent comprises a packaging component which is a particle, a viral vector, a virus, or a virus-like particle.

In embodiments, the composition further comprises an immunosuppressant.

In another aspect, the present disclosure provides a pharmaceutical composition comprising an immune tolerizing effective amount of a heat shock protein for treating a cardiovascular, endocrine, gastrointestinal, genetic, hematologic, infectious, metabolic, neurological/psychiatric, oncological (e.g., cancer), ophthalmologic, respiratory, and/or urological disease or disorder.

It shall be understood that different aspects and/or embodiments of the invention can be appreciated individually, collectively, or in combination with each other. Various aspects and/or embodiments of the invention described herein may be applied to any of the uses set forth below and in other methods for increasing lifespan in a mammal. Any description herein concerning a specific composition and/or method apply to and may be used for any other specific composition and/or method as disclosed herein. Additionally, any composition disclosed herein is applicable to any herein-disclosed method. In other words, any aspect or embodiment described herein can be combined with any other aspect or embodiment as disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a scheme of one embodiment of the disclosed method for inducing immunotolerance in a subject.

FIG. 2 illustrates a mechanism of converting naive T-cells into Tregs resulting in suppressed immune function in a subject.

FIG. 3A illustrates αA-crystallin's (CRYAA) and αB-crystallin's (CRYAB) effect on cytokine secretion from human dendritic cells.

FIG. 3B illustrates αA-crystallin's (CRYAA) and αB-crystallin's (CRYAB) effect on cytokine secretion from human M1 macrophages.

FIG. 4 illustrates the induction of regulatory T-cells in αB-crystallin (CRYAB) peptide-treated dendritic cell co-culture.

FIG. 5 illustrates AAV gene delivery into HEK 293 cells. HEK 293 cells were transduced with AAV1-GFP, in the presence and absence of αB-crystallin. The average number of GFP-expressing HEK 293 cells per randomly-selected 200× field is shown.

FIG. 6 illustrates anti-AAV1 antibody titers in mice immunized with AAV1, in the presence or absence of αB-crystallin. The data shown represents the averaged anti-AAV1 antibody titers from each group, with 5 mice per group. Error bars represent standard error of the mean.

FIG. 7 illustrates relative neutralizing antibody titers in the pooled sera from mice previously immunized with PBS, αB-crystallin (CRYAB), AAV1, or a combination of αB-crystallin and AAV1 determined by the transduction of HEK 293 cells with AAV1-GFP. Presented are representative histograms from each treatment group.

FIG. 8 illustrates the average number of GFP expressing HEK 293 cells in each treatment group after transduction with AAV1-GFP, as measured by flow cytometry. Error bars represent standard deviation.

FIG. 9 illustrates the induction of regulatory T-cells in αB-crystallin (CRYAB)-treated dendritic cell co-cultures in the presence of AAV1.

FIG. 10A illustrates a transgene re-dosing study design and schedule.

FIG. 10B shows anti-AAV8 IgG titers at day 14 following AAV8 immunization in mice of the transgene re-dosing study. Anti-AAV8 IgG in the serum of the mice was measured by ELISA. Average antibody titers plus standard deviations are plotted for each group.

FIG. 10C shows radiance measurements for luciferase activity at day 70 from immunized mice in the transgene re-dosing study. The average radiance from mice of each group, captured from both the dorsal and ventral positions are shown. Error bars indicate standard deviation.

FIG. 11A illustrates a dose-ratio study design and schedule.

FIG. 11B shows anti-AAV8 IgG titers at day 28 following tolerance induction with 10⁵ vector genomes (VG) of AAV8 and various amounts of αB-crystallin in mice of the dose-ratio study.

FIG. 12A illustrates the design and schedule for a tolerization towards IgG study.

FIG. 12B shows anti-human IgG titer at day 14 in the presence or absence of αB-crystallin in mice of the tolerization towards IgG study.

FIG. 12C shows anti-human IgG titer at day 21 in the presence or absence of αB-crystallin in mice of the tolerization towards IgG study.

### DETAILED DESCRIPTION OF THE INVENTION

The present disclosure is based, in part, on the discovery of methods and compositions that overcome immunogenicity of therapeutic agents and promote immunotolerance. Thus, a subject may be treated with a higher dose of therapeutic agent, multiple doses, and/or for a longer duration than would be possible otherwise due to adverse immune reactions. Alternately, a subject may be treated with a lower dose of therapeutic agent and/or for a shorter duration than would be standard due to increased efficacy of the therapeutic agent. Additionally, the present disclosure provides use of therapeutic agents that were previously disfavored due to adverse immune reactions. Accordingly, the methods and compositions provided both a therapeutic effect and an immune tolerizing effect, which together better treat a subject in need, and provide an improved safety profile.

An aspect of the present disclosure is a method for generating an immune tolerizing effect against a therapeutic agent administered to a subject. The method comprises administering to the subject a therapeutic agent and administering to the subject an effective amount of a heat shock protein to generate an immune tolerizing effect against the therapeutic agent.

In embodiments, the therapeutic agent is administered concurrently with the administering of the heat shock protein, the therapeutic agent is administered to the subject before the administering of the heat shock protein, or the therapeutic agent is administered to the subject after the administering of the heat shock protein.

In embodiments, the immune tolerizing effect comprises an increased amount of antigen-specific regulatory T cells (Tregs) in the subject. The antigen-specific Tregs recognize the therapeutic agent or a portion thereof. In embodiments, the immune tolerizing effect results in an increased amount of an expression level of CD25 and/or FoxP3 on the antigen-specific Tregs.

In embodiments, the administering to the subject a therapeutic agent comprises administering one or more first low doses of the therapeutic agent and followed by one or more doses of the therapeutic agent at a higher amount (e.g., such as a therapeutically effective amount). In embodiments, the heat shock protein is administered concurrently with the one or more first low doses of the therapeutic agent; the heat shock protein is administered before the one or more first low doses of the therapeutic agent; the heat shock protein is administered after the one or more first low doses of the therapeutic agent; or the heat shock protein is administered before and after the one or more first low doses of the therapeutic agent. In embodiments, the heat shock protein is also administered before, concurrently with, or followed by the one or more doses of the therapeutic agent administered at a higher amount, such as a therapeutically effective amount. In some embodiments, the one or more low doses are at a dose that is presumed to be a sub-effective amount. In some embodiments, the one or more low doses are at a level where the therapeutic effect may be less than could be achieved with a higher dose.

The therapeutic agent may be administered before, during, and/or after the onset of disease or injury and/or administered prophylactically. A therapeutic agent comprises an active agent, such as a nucleic acid, a peptide, a protein, a compound, a chemotherapeutic, a cell, or any combination thereof. A therapeutic agent can optionally include a delivery vehicle, e.g., a packaging component, for the active agent. The packaging component may be a virus particle, a non-viral particle, a polymer coating or a molecule co-administered with the active agent. A therapeutic agent also can include an active agent without a packaging component. In some cases, a therapeutic agent can induce an immune response in a subject and such response can be directed to the therapeutic agent or a portion thereof. An immune response can be directed to the packaging component (or a portion or component thereof), to the active agent or a portion thereof, or to a combination of the packaging component and the active agent. In some cases, a therapeutic agent comprises an active agent and is administered without a packaging component, and in such cases an immune response can be directed to the active agent or a portion thereof.

An immune tolerizing effect (also referred to herein as immune tolerance) includes the suppression of an immune response and/or the activation of immune tolerance to one or more antigens and can include one or more of the reduction, inhibition and/or prevention of the generation of antibodies against an antigen, a reduction in or inhibition of inflammatory cytokines, an increase in or generation of anti-inflammatory cytokines, and/or an increase in or generation of antigen-specific T regulatory cells (Tregs). In embodiments, a composition of the present invention or a method comprising the same, provides immune tolerizing effect which comprises a reduction of the amount of an anti-therapeutic-agent antibody expressed/synthesized in the subject.

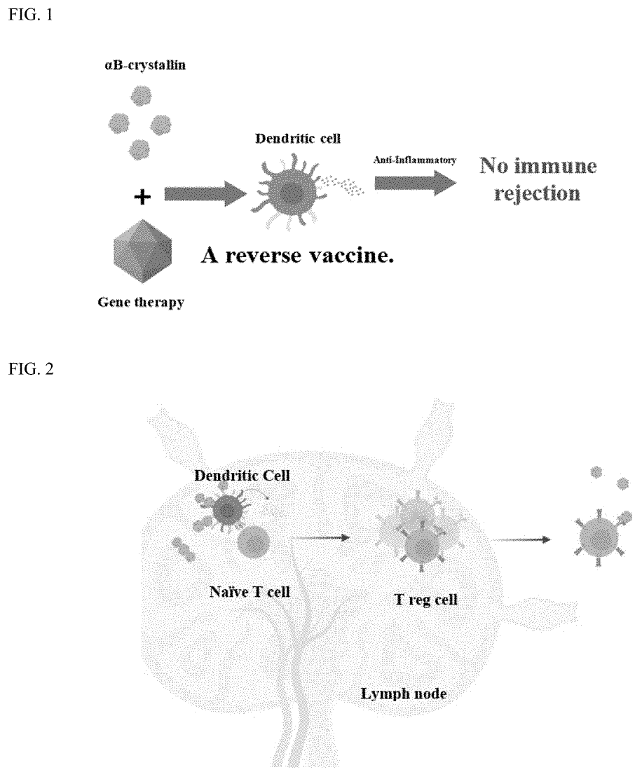

FIG. 1 illustrates a scheme of an embodiment of the disclosed method for inducing immunotolerance in a subject. As shown, a therapeutic agent (here, a viral vector used in a gene therapy) and an agent that promotes immune tolerance (here, αB-crystallin) are administered. If the therapeutic agent is administered alone, the subject's immune system would become activated and generate an immune response against the therapeutic agent. However, when both a therapeutic agent and an agent that promotes immune tolerance are administered, the subject's immune system does not become activated against the therapeutic agent or the activation is favorably reduced. It could be said that the present methods and compositions are the opposite of a vaccine in that the co-therapy does not increase immune response but instead reduces an immune response to the therapeutic agent.

In addition to avoiding an increase in an immune response against the therapeutic agent, the present methods and compositions instead suppress the immune response. FIG. 2 illustrates a mechanism through which the present methods and compositions promote development of naive T-cells into regulatory T cells (Tregs) that suppress immune activity in a subject.

### Heat Shock Proteins

The present methods and compositions comprise at least one heat shock protein (HSP), or a functional fragment thereof, that induces immunotolerance in a subject.

HSPs are generally defined as molecular chaperones which bind unfolded protein and promote proper re-folding. This class of proteins is diverse in terms of their size, subcellular localization, and functional mechanisms and members are found in all kingdoms of life. Generally, HSPs are grouped into categories based on their molecular weight. Major groups include the HSP90s, HSP70s and the small HSPs, such as HSP27 and αB-crystallin (CRYAB). Many HSP genes are stress inducible, which allows for the rapid accumulation of HSPs in response to protein denaturing stresses such as heavy metal stress, hypoxia and the eponymous heat shock. However, HSPs have long been known to function outside this canonical role, instead serving as constitutive aids in the proper folding of newly translated polypeptides or as molecular motors for protein translocation. Furthermore, the accumulation of some classical, stress-inducible, chaperone-type HSPs is now known to have effects on a variety of cell signaling pathways. For instance, both HSP70 family proteins and HSP27 are known to inhibit pro-apoptotic signaling. In the present disclosure, HSPs are used as inducers of tolerogenic mechanisms, such as promoting immunotolerance, activating Tregs (a type of helper T cell aiding in suppressing the immune response to self-antigens) and tolerogenic DCs (a class of antigen presenting cells which generally serve to promote an immunotolerant response), etc.

Provided herein are compositions that comprise at least one heat shock protein or a functional fragment thereof, which when delivered in connection with a therapeutic agent, provide an immunotolerance or otherwise reduce, inhibit, or prevent an immune reaction to the therapeutic agent.

In embodiments, the heat shock protein is αB-crystallin (CRYAB), αA-crystallin (CRYAA), HSP60, HSP70, HSP72, HSP84, HSP90, HSP104, GP96, HSP33, HSP27, HSP22, HSP20, HSP12, HSP10, HSP7, or a functional fragment thereof.

In embodiments, the heat shock protein is CRYAB or CRYAA.

In embodiments, the CRYAB comprises a sequence selected from the group consisting of SEQ ID NO: 18-25. In embodiments, the CRYAB comprises a sequence of SEQ ID NO: 18. In embodiments, the CRYAB comprises a sequence that is at least 65%, 70%, 75%, 80%, 85%, 90%, 95%, 96%, 97%, 98%, 99% identical to SEQ ID NO:18. In embodiments, the functional fragment of CRYAB is selected from the group consisting of SEQ ID NO: 46 to SEQ ID NO: 48. In embodiments, the functional fragment of CRYAB is at least 5 amino acids in length, e.g., at least 5, 6, 7, 8, 9, 10, 11, 12, 13, 14, 15, 16, 17, 18, 19, 20, 21, 25, 30, 40, 50, 60, 70, 80, 90, 100, or 125, and any number of amino acids therebetween.

In embodiments, the CRYAA comprises a sequence selected from the group consisting of SEQ ID NO: 26-33. In embodiments, the CRYAA comprises a sequence of SEQ ID NO: 26. In embodiments, the CRYAB comprises a sequence that is at least 65%, 70%, 75%, 80%, 85%, 90%, 95%, 96%, 97%, 98%, 99% identical to SEQ ID NO:26. In embodiments, the functional fragment of CRYAA is at least 10 amino acids in length, e.g., at least 10, 11, 12, 13, 14, 15, 16, 17, 18, 19, 20, 21, 25, 30, 40, 50, 60, 70, 80, 90, 100, or 125, and any number of amino acids there between.

In embodiments, the at least one heat shock protein or a functional fragment thereof is a member of a class of heat shock proteins known as small heat shock proteins (sHsp). In embodiments, the sHsp comprises one or more features selected from (i) a subunit molecular mass between about 12 and about 43 kDa, (ii) an α-crystallin domain, (iii) an N-terminal domain and (iv) C-terminal extension.

In embodiments, the compositions, and methods using the same, disclosed herein comprise at least one heat shock protein or a functional fragment thereof, which comprises a peptide sequence of any of SEQ ID NOs: 1-52 shown below in TABLE 1. In embodiments, a heat shock protein comprises a sequence that has at least 80% sequence identity (e.g., at least 85%, 86%, 87%, 88%, 89%, 90%, 91%, 92%, 93%, 94%, 95%, 96%, 97%, 98%, or 99% sequence identity) to any one of SEQ ID NOs: 1-52. In embodiments, a composition comprises a functional fragment of any one of SEQ ID NOs: 1-52, or a functional fragment of a sequence having at least 80% sequence identity (e.g., at least 85%, 86%, 87%, 88%, 89%, 90%, 91%, 92%, 93%, 94%, 95%, 96%, 97%, 98%, or 99% sequence identity) to any one of SEQ ID NOs: 1-52, wherein the fragment is at least 5 amino acids in length, e.g., at least 5, 6, 7, 8, 9, 10, 11, 12, 13, 14, 15, 16, 17, 18, 19, 20, 21, 25, 30, 40, 50, 60, 70, 80, 90, 100, 125, 150, 175, 200, 225, 250, 275, 300, 325, or 340, and any number amino acids therebetween. Illustrative fragments have an amino acid sequence of any one of SEQ ID NO: 46-52.

In embodiments, a composition, and methods using the same, comprises at least one heat shock protein, e.g., a combination of heat shock proteins comprising a peptide sequence of any of SEQ ID NOs: 1-52. In embodiments, the at least one heat shock protein comprises a combination of functional fragments of the heat shock proteins, wherein each of the function fragments comprises a fragment of any one of SEQ ID NOs: 1-52. In embodiments, the at least one heat shock protein comprises a combination of heat shock proteins and functional fragments thereof, wherein each of the heat shock protein comprises a peptide sequence of any of SEQ ID NOs: 1-52 and each of the functional fragments comprises a fragment of any one of SEQ ID NOs: 1-52.

In embodiments, the composition, and methods using the same, comprises a heat shock protein comprising a sequence of any of SEQ ID NOs: 18-33, or a functional fragment of a heat shock protein comprising a fragment of a sequence of any of SEQ ID NOs: 18-33, e.g., a fragment comprising the sequence of SEQ ID NO: 46-52.

TABLE 1
Illustrative Polypeptide Sequences

| SEQ | | |
|---|---|---|
| ID | | |
| NO | NAME | Sequence (N-TERMINUS TO C-TERMINUS) |
| 1 | Human | MSVVGLDVGSQSCYIAVARAGGIETIANEFSDRCTPSVISFGSKNRTIGVAAKNQ |
| | HSP110 | QITHANNTVSNFKRFHGRAFNDPFIQKEKENLSYDLVPLKNGGVGIKVMYMGEE |
| | | HLFSVEQITAMLLTKLKETAENSLKKPVTDCVISVPSFFTDAERRSVLDAAQIVGL |
| | | NCLRLMNDMTAVALNYGIYKQDLPSLDEKPRIVVFVDMGHSAFQVSACAFNKG |
| | | KLKVLGTAFDPFLGGKNFDEKLVEHFCAEFKTKYKLDAKSKIRALLRLYQECEK |
| | | LKKLMSSNSTDLPLNIECFMNDKDVSGKMNRSQFEELCAELLQKIEVPLYSLLEQ |
| | | THLKVEDVSAVEIVGGATRIPAVKERIAKFFGKDISTTLNADEAVARGCALQCAI |
| | | LSPAFKVREFSVTDAVPFPISLIWNHDSEDTEGVHEVFSRNHAAPFSKVLTFLRRG |
| | | PFELEAFYSDPQGVPYPEAKIGRFVVQNVSAQKDGEKSRVKVKVRVNTHGIFTIS |
| | | TASMVEKVPTEENEMSSEADMECLNQRPPENPDTDKNVQQDNSEAGTQPQVQT |
| | | DAQQTSQSPPSPELTSEENKIPDADKANEKKVDQPPEAKKPKIKVVNVELPIEAN |
| | | LVWQLGKDLLNMYIETEGKMIMQDKLEKERNDAKNAVEEYVYEFRDKLCGPY |
| | | EKFICEQDHQNFLRLLTETEDWLYEEGEDQAKQAYVDKLEELMKIGTPVKVRFQ |
| | | EAEERPKMFEELGQRLQHYAKIAADFRNKDEKYNHIDESEMKKVEKSVNEVME |
| | | WMNNVMNAQAKKSLDQDPVVRAQEIKTKIKELNNTCEPVVTQPKPKIESPKLER |
| | | TPNGPNIDKKEEDLEDKNNFGAEPPHQNGECYPNEKNSVNMDLD |
| 2 | Mouse | MSVVGLDVGSQSCYIAVARAGGIETIANEFSDRCTPSVISFGSKNRTIGVAAKNQ |
| | HSP110 | QITHANNTVSSFKRFHGRAFNDPFIQKEKENLSYDLVPMKNGGVGIKVMYMDEE |
| | | HFFSVEQITAMLLTKLKETAENNLKKPVTDCVISVPSFFTDAERRSVLDAAQIVG |
| | | LNCLRLMNDMTAVALNYGIYKQDLPNAEEKPRVVVFVDMGHSSFQVSACAFN |
| | | KGKLKVLGTAFDPFLGGKNFDEKLVEHFCAEFKTKYKLDAKSKIRALLRLHQEC |
| | | EKLKKLMSSNSTDLPLNIECFMNDKDVSGKMNRSQFEELCAELLQKIEVPLHSL |
| | | MAQTQLKAEDVSAIEIVGGATRIPAVKERIAKFFGKDVSTTLNADEAVARGCAL |
| | | QCAILSPAFKVREFSVTDAVPFPISLVWNHDSEETEGVHEVFSRNHAAPFSKVLTF |
| | | LRRGPFELEAFYSDPQGVPYPEAKIGRFVVQNVSAQKDGEKSRVKVKVRVNTH |
| | | GIFTISTASMVEKVPTEEEDGSSLEADMECPNQRPTESSDVDKNIQQDNSEAGTQ |
| | | PQVQTDGQQTSQSPPSPELTSEESKTPDADKANEKKVDQPPEAKKPKIKVVNVEL |
| | | PVEANLVWQLGRDLLNMYIETEGKMIMQDKLEKERNDAKNAVEECVYEFRDK |
| | | LCGPYEKFICEQEHEKFLRLLTETEDWLYEEGEDQAKQAYIDKLEELMKMGTPV |
| | | KVRFQEAEERPKVLEELGQRLQHYAKIAADFRGKDEKYNHIDESEMKKVEKSV |
| | | NEVMEWMNNVMNAQAKRSLDQDPVVRTHEIRAKVKELNNVCEPVVTQPKPKI |
| | | ESPKLERTPNGPNIDKKEDLEGKNNLGAEAPHQNGECHPNEKGSVNMDLD |
| 3 | Rat | MSVVGLDVGSQSCYIAVARAGGIETIANEFSDRCTPSVISFGPKNRTIGVAAKNQ |
| | HSP110 | QITHANNTVSSFKRFHGRAFNDPFIQKEKENLSYDLVPMKNGGVGIKVMYMDE |
| | | DHLFSVEQITAMLLTKLKETAENNLKKPVTDCVISVPSFFTDAERRSVLDAAQIV |
| | | GLNCLRLMNDMTAVALNYGIYKQDLPNADEKPRVVVFVDMGHSSFQVSACAF |
| | | NKGKLKVLGTAFDPFLGGKNFDEKLVEHFCAEFKTKYKLDAKSKIRALLRLHQE |
| | | CEKLKKLMSSNSTDLPLNIECFMNDKDVSAKMNRSQFEELCAELLQKIEVPLHLL |
| | | MEQTHLKTEEVSAIEIVGGATRIPAVKERIARFFGKDVSTTLNADEAVARGCALQ |
| | | CAILSPAFKVREFSVTDAVPFPISLVWNHDSEETEGVHEVFSRNHAAPFSKVLTFL |
| | | RRGPFELEAFYSDPQAVPYPEAKIGRFVVQNVSAQKDGEKSKVKVKVRVNTHGI |
| | | FTISTASMVEKVPTEEEDGSSVEADMECPNQKPAESSDVDKNIQQDNSEAGTQP |
| | | QVQTDGQQTSQSPPSPELTSEENKIPDADKANEKKVDQPPEAKKPKIKVVNVELP |
| | | VEANLVWQLGRDLLNMYIEIEGKMIMQDKLEKERNDAKNAVEECVYEFRDKL |
| | | CGPYEKFICEQEHEKFLRLLTETEDWLYEEGEDQAKQAYIDKLEELMKMGTPVK |
| | | VRFQEAEERPRVLEELGQRLQHYAKIAADFRGKDEKYNHIDESEMKKVEKSVNE |
| | | VMEWMNNVMNAQAKRSLHQDPVVRTHEISAKVKELNNVCEPVVTQPKPKIESP |
| | | KLERTPNGPNMDKKEDLEGKSNLGADAPHQNGECHPNEKGSVSMDLD |
| 4 | Human | MPEEVHHGEEEVETFAFQAEIAQLMSLIINTFYSNKEIFLRELISNASDALDKIRYE |
| | HSP90 | SLTDPSKLDSGKELKIDIIPNPQERTLTLVDTGIGMTKADLINNLGTIAKSGTKAF |
| | | MEALQAGADISMIGQFGVGFYSAYLVAEKVVVITKHNDDEQYAWESSAGGSFT |
| | | VRADHGEPIGRGTKVILHLKEDQTEYLEERRVKEVVKKHSQFIGYPITLYLEKER |
| | | EKEISDDEAEEEKGEKEEEDKDDEEKPKIEDVGSDEEDDSGKDKKKKTKKIKEK |
| | | YIDQEELNKTKPIWTRNPDDITQEEYGEFYKSLTNDWEDHLAVKHFSVEGQLEF |
| | | RALLFIPRRAPFDLFENKKKKNNIKLYVRRVFIMDSCDELIPEYLNFIRGVVDSED |
| | | LPLNISREMLQQSKILKVIRKNIVKKCLELFSELAEDKENYKKFYEAFSKNLKLGI |
| | | HEDSTNRRRLSELLRYHTSQSGDEMTSLSEYVSRMKETQKSIYYITGESKEQVAN |
| | | SAFVERVRKRGFEVVYMTEPIDEYCVQQLKEFDGKSLVSVTKEGLELPEDEEEK |
| | | KKMEESKAKFENLCKLMKEILDKKVEKVTISNRLVSSPCCIVTSTYGWTANMER |
| | | IMKAQALRDNSTMGYMMAKKHLEINPDHPIVETLRQKAEADKNDKAVKDLVV |
| | | LLFETALLSSGFSLEDPQTHSNRIYRMIKLGLGIDEDEVAAEEPNAAVPDEIPPLEG |
| | | DEDASRMEEVD |
| 5 | Rat | MPEETQTQDQPMEEEEVETFAFQAEIAQLMSLIINTFYSNKEIFLRELISNSSDALD |
| | HSP90- | KIRYESLTDPSKLDSGKELHINLIPNKQDRTLTIVDTGIGMTKADLINNLGTIAKSG |
| | alpha | TKAFMEALQAGADISMIGQFGVGFYSAYLVAEKVTVITKHNDDEQYAWESSAG |
| | | GSFTVRTDTGEPMGRGTKVILHLKEDQTEYLEERRIKEIVKKHSQFIGYPITLFVE |
| | | KERDKEVSDDEAEEKEEKEEEKEKEEKESDDKPEIEDVGSDEEEEEKKDGDKKK |
| | | KKKIKEKYIDQEELNKTKPIWTRNPDDITNEEYGEFYKSLTNDWEEHLAVKHFS |
| | | VEGQLEFRALLFVPRRAPFDLFENRKKKNNIKLYVRRVFIMDNCEELIPEYLNFIR |
| | | GVVDSEDLPLNISREMLQQSKILKVIRKNLVKKCLELFTELAEDKENYKKFYEQF |
| | | SKNIKLGIHEDSQNRKKLSELLRYYTSASGDEMVSLKDYCTRMKENQKHIYFITG |
| | | ETKDQVANSAFVERLRKHGLEVIYMIEPIDEYCVQQLKEFEGKTLVSVTKEGLEL |
| | | PEDEEEKKKQEEKKTKFENLCKIMKDILEKKVEKVVVSNRLVTSPCCIVTSTYG |
| | | WTANMERIMKAQALRDNSTMGYMAAKKHLEINPDHSIIETLRQKAEADKNDKS |
| | | VKDLVILLYETALLSSGFSLEDPQTHANRIYRMIKLGLGIDEDDPTVDDTSAAVT |
| | | EEMPPLEGDDDTSRMEEVD |
| 6 | Rat | MPEEVHHGEEEVETFAFQAEIAQLMSLIINTFYSNKEIFLRELISNASDALDKIRYE |
| | HSP90- | SLTDPSKLDSGKELKIDIIPNPQERTLTLVDTGIGMTKADLINNLGTIAKSGTKAF |
| | beta | MEALQAGADISMIGQFGVGFYSAYLVAEKVVVITKHNDDEQYAWESSAGGSFT |
| | | VRADHGEPIGRGTKVILHLKEDQTEYLEERRVKEVVKKHSQFIGYPITLYLEKER |
| | | EKEISDDEAEEEKGEKEEEDKEDEEKPKIEDVGSDEEDDSGKDKKKKTKKIKEK |
| | | YIDQEELNKTKPIWTRNPDDITQEEYGEFYKSLTNDWEDHLAVKHFSVEGQLEF |
| | | RALLFIPRRAPFDLFENNIKLYVRRVFIMDSCDELIPEYLNFIRGVVDSED |
| | | LPLNISREMLQQSKILKVIRKNIVKKCLELFSELAEDKENYKKFYEAFSKNLKLGI |
| | | HEDSTNRRRLSELLRYHTSQSGDEMTSLSEYVSRMKETQKSIYYITGESKEQVAN |
| | | SAFVERVRKRGFEVVYMTEPIDEYCVQQLKEFDGKSLVSVTKEGLELPEDEEEK |
| | | KKMEESKAKFENLCKLMKEILDKKVEKVTISNRLVSSPCCIVTSTYGWTANMER |
| | | IMKAQALRDNSTMGYMMAKKHLEINPDHPIVETLRQKAEADKNDKAVKDLVV |
| | | LLFETALLSSGFSLEDPQTHSNRIYRMIKLGLGIDEDEVTAEEPSAAVPDEIPPLEG |
| | | DEDASRMEEVD |
| 7 | Yeast | MASETFEFQAEITQLMSLIINTVYSNKEIFLRELISNASDALDKIRYKSLSDPKQLE |
| | HSP90 | TEPDLFIRITPKPEQKVLEIRDSGIGMTKAELINNLGTIAKSGTKAFMEALSAGAD |
| | | VSMIGQFGVGFYSLFLVADRVQVISKSNDDEQYIWESNAGGSFTVTLDEVNERIG |
| | | RGTILRLFLKDDQLEYLEEKRIKEVIKRHSEFVAYPIQLVVTKEVEKEVPIPEEEK |
| | | KDEEKKDEEKKDEDDKKPKLEEVDEEEEKKPKTKKVKEEVQEIEELNKTKPLW |
| | | TRNPSDITQEEYNAFYKSISNDWEDPLYVKHFSVEGQLEFRAILFIPKRAPFDLFES |
| | | KKKKNNIKLYVRRVFITDEAEDLIPEWLSFVKGVVDSEDLPLNLSREMLQQNKI |
| | | MKVIRKNIVKKLIEAFNEIAEDSEQFEKFYSAFSKNIKLGVHEDTQNRAALAKLL |
| | | RYNSTKSVDELTSLTDYVTRMPEHQKNIYYITGESLKAVEKSPFLDALKAKNFEV |
| | | LFLTDPIDEYAFTQLKEFEGKTLVDITKDFELEETDEEKAEREKEIKEYEPLTKAL |
| | | KEILGDQVEKVVVSYKLLDAPAAIRTGQFGWSANMERIMKAQALRDSSMSSYM |
| | | SSKKTFEISPKSPIIKELKKRVDEGGAQDKTVKDLTKLLYETALLTSGFSLDEPTSF |
| | | ASRINRLISLGLNIDEDEETETAPEASTAAPVEEVPADTEMEEVD |
| 8 | Human | MAKAAAIGIDLGTTYSCVGVFQHGKVEIIANDQGNRTTPSYVAFTDTERLIGDAA |
| | HSP70- | KNQVALNPQNTVFDAKRLIGRKFGDPVVQSDMKHWPFQVINDGDKPKVQVSY |
| | 1A | KGETKAFYPEEISSMVLTKMKEIAEAYLGYPVTNAVITVPAYFNDSQRQATKDA |
| | | GVIAGLNVLRIINEPTAAAIAYGLDRTGKGERNVLIFDLGGGTFDVSILTIDDGIFE |
| | | VKATAGDTHLGGEDFDNRLVNHFVEEFKRKHKKDISQNKRAVRRLRTACERAK |
| | | RTLSSSTQASLEIDSLFEGIDFYTSITRARFEELCSDLFRSTLEPVEKALRDAKLDK |
| | | AQIHDLVLVGGSTRIPKVQKLLQDFFNGRDLNKSINPDEAVAYGAAVQAAILMG |
| | | DKSENVQDLLLLDVAPLSLGLETAGGVMTALIKRNSTIPTKQTQIFTTYSDNQPG |
| | | VLIQVYEGERAMTKDNNLLGRFELSGIPPAPRGVPQIEVTFDIDANGILNVTATD |
| | | KSTGKANKITITNDKGRLSKEEIERMVQEAEKYKAEDEVQRERVSAKNALESYA |
| | | FNMKSAVEDEGLKGKISEADKKKVLDKCQEVISWLDANTLAEKDEFEHKRKEL |
| | | EQVCNPIISGLYQGAGGPGPGGFGAQGPKGGSGSGPTIEEVD |
| 9 | Human | MAKAAAIGIDLGTTYSCVGVFQHGKVEIIANDQGNRTTPSYVAFTDTERLIGDAA |
| | HSP70- | KNQVALNPQNTVFDAKRLIGRKFGDPVVQSDMKHWPFQVINDGDKPKVQVSY |
| | 1B | KGETKAFYPEEISSMVLTKMKEIAEAYLGYPVTNAVITVPAYFNDSQRQATKDA |
| | | GVIAGLNVLRIINEPTAAAIAYGLDRTGKGERNVLIFDLGGGTFDVSILTIDDGIFE |
| | | VKATAGDTHLGGEDFDNRLVNHFVEEFKRKHKKDISQNKRAVRRLRTACERAK |
| | | RTLSSSTQASLEIDSLFEGIDFYTSITRARFEELCSDLFRSTLEPVEKALRDAKLDK |
| | | AQIHDLVLVGGSTRIPKVQKLLQDFFNGRDLNKSINPDEAVAYGAAVQAAILMG |
| | | DKSENVQDLLLLDVAPLSLGLETAGGVMTALIKRNSTIPTKQTQIFTTYSDNQPG |
| | | VLIQVYEGERAMTKDNNLLGRFELSGIPPAPRGVPQIEVTFDIDANGILNVTATD |
| | | KSTGKANKITITNDKGRLSKEEIERMVQEAEKYKAEDEVQRERVSAKNALESYA |
| | | FNMKSAVEDEGLKGKISEADKKKVLDKCQEVISWLDANTLAEKDEFEHKRKEL |
| | | EQVCNPIISGLYQGAGGPGPGGFGAQGPKGGSGSGPTIEEVD |
| 10 | Mouse | MAKNTAIGIDLGTTYSCVGVFQHGKVEIIANDQGNRTTPSYVAFTDTERLIGDAA |
| | HSP70- | KNQVALNPQNTVFDAKRLIGRKFGDAVVQSDMKHWPFQVVNDGDKPKVQVN |
| | 1A | YKGESRSFFPEEISSMVLTKMKEIAEAYLGHPVTNAVITVPAYFNDSQRQATKDA |
| | | GVIAGLNVLRIINEPTAAAIAYGLDRTGKGERNVLIFDLGGGTFDVSILTIDDGIFE |
| | | VKATAGDTHLGGEDFDNRLVSHFVEEFKRKHKKDISQNKRAVRRLRTACERAK |
| | | RTLSSSTQASLEIDSLFEGIDFYTSITRARFEELCSDLFRGTLEPVEKALRDAKMDK |
| | | AQIHDLVLVGGSTRIPKVQKLLQDFFNGRDLNKSINPDEAVAYGAAVQAAILMG |
| | | DKSENVQDLLLLDVAPLSLGLETAGGVMTALIKRNSTIPTKQTQTFTTYSDNQPG |
| | | VLIQVYEGERAMTRDNNLLGRFELSGIPPAPRGVPQIEVTFDIDANGILNVTATDK |
| | | STGKANKITITNDKGRLSKEEIERMVQEAERYKAEDEVQRDRVAAKNALESYAF |
| | | NMKSAVEDEGLKGKLSEADKKKVLDKCQEVISWLDSNTLADKEEFVHKREELE |
| | | RVCSPIISGLYQGAGAPGAGGFGAQAPKGASGSGPTIEEVD |
| 11 | Mouse | MAKNTAIGIDLGTTYSCVGVFQHGKVEIIANDQGNRTTPSYVAFTDTERLIGDAA |
| | HSP70- | KNQVALNPQNTVFDAKRLIGRKFGDAVVQSDMKHWPFQVVNDGDKPKVQVN |
| | 1B | YKGESRSFFPEEISSMVLTKMKEIAEAYLGHPVTNAVITVPAYFNDSQRQATKDA |
| | | GVIAGLNVLRIINEPTAAAIAYGLDRTGKGERNVLIFDLGGGTFDVSILTIDDGIFE |
| | | VKATAGDTHLGGEDFDNRLVSHFVEEFKRKHKKDISQNKRAVRRLRTACERAK |
| | | RTLSSSTQASLEIDSLFEGIDFYTSITRARFEELCSDLFRGTLEPVEKALRDAKMDK |
| | | AQIHDLVLVGGSTRIPKVQKLLQDFFNGRDLNKSINPDEAVAYGAAVQAAILMG |
| | | DKSENVQDLLLLDVAPLSLGLETAGGVMTALIKRNSTIPTKQTQTFTTYSDNQPG |
| | | VLIQVYEGERAMTRDNNLLGRFELSGIPPAPRGVPQIEVTFDIDANGILNVTATDK |
| | | STGKANKITITNDKGRLSKEEIERMVQEAERYKAEDEVQRDRVAAKNALESYAF |
| | | NMKSAVEDEGLKGKLSEADKKKVLDKCQEVISWLDSNTLADKEEFVHKREELE |
| | | RVCSPIISGLYQGAGAPGAGGFGAQAPPKGASGSGPTIEEVD |
| 12 | Human | MGKDYYQTLGLARGASDEEIKRAYRRQALRYHPDKNKEPGAEEKFKEIAEAYD |
| | HSP40 | VLSDPRKREIFDRYGEEGLKGSGPSGGSGGGANGTSFSYTFHGDPHAMFAEFFG |
| | | GRNPFDTFFGQRNGEEGMDIDDPFSGFPMGMGGFTNVNFGRSRSAQEPARKKQ |
| | | DPPVTHDLRVSLEEIYSGCTKKMKISHKRLNPDGKSIRNEDKILTIEVKKGWKEG |
| | | TKITFPKEGDQTSNNIPADIVFVLKDKPHNIFKRDGSDVIYPARISLREALCGCTV |
| | | NVPTLDGRTIPVVFKDVIRPGMRRKVPGEGLPLPKTPEKRGDLIIEFEVIFPERIPQ |
| | | TSRTVLEQVLPI |
| 13 | Mouse | MGKDYYQTLGLARGASDDEIKRAYRRQALRYHPDKNKEPGAEEKFKEIAEAYD |
| | HSP40 | VLSDPRKREIFDRYGEEGLKGGSPSGGSSGGANGTSFSYTFHGDPHAMFAEFFGG |
| | | RNPFDTFFGQRNGEEGMDIDDTFSSFPMGMGGFTNMNFGRSRPSQEPTRKKQDP |
| | | PVTHDLRVSLEEIYSGCTKKMKISHKRLNPDGKSIRNEDKILTIEVKRGWKEGTKI |
| | | TFPKEGDQTSNNIPADIVFVLKDKPHNIFKRDGSDVIYPARISLREALCGCTVNVP |
| | | TLDGRTIPVVFKDVIRPGMRRKVPGEGLPLPKTPEKRGDLVIEFEVIFPERIPVSSR |
| | | TILEQVLPI |
| 14 | Rat | MVDYYEVLGVQRHASPEDIKKAYRKQALKWHPDKNPENKEEAERKFKQVAEA |
| | HSP40 | YEVLSDAKKRDIYDKYGKEGLNGGGGGGGSHFDSPFEFGFTFRNPDDVFREFFG |
| | | GRDPFSFDFFEDPFDDFFGNRRGPRGSRSRGAGSFFSAFSGFPSFGSGFPAFDTGFT |
| | | PFGSLGHGGLTSFSSASFGGSGMGNFKSISTSTKIVNGKKITTKRIVENGQERVEV |
| | | EEDGQLKSLTINGVADENALAEECRRRGQPTPALAPGPAPAPARVPSQARPPTPA |
| | | PTPAPAQTPAPSVSTRPQKPPRPAPTAKLVSKSNWEDEEQDRQRVPGNCDAPMT |
| | | SAGLKEGGKRKKQKQKEDSKKKKSTKGNH |
| 15 | Human | MTERRVPFSLLRGPSWDPFRDWYPHSRLFDQAFGLPRLPEEWSQWLGGSSWPG |
| | HSP27 | YVRPLPPAAIESPAVAAPAYSRALSRQLSSGVSEIRHTADRWRVSLDVNHFAPDE |
| | | LTVKTKDGVVEITGKHEERQDEHGYISRCFTRKYTLPPGVDPTQVSSSLSPEGTL |
| | | TVEAPMPKLATQSNEITIPVTFESRAQLGGPEAAKSDETAAK |
| 16 | Rat | MTERRVPFSLLRSPSWEPFRDWYPAHSRLFDQAFGVPRFPDEWSQWFSSAGWPG |
| | HSP27 | YVRPLPAATAEGPAAVTLARPAFSRALNRQLSSGVSEIRQTADRWRVSLDVNHF |
| | | APEELTVKTKEGVVEITGKHEERQDEHGYISRCFTRKYTLPPGVDPTLVSSSLSPE |
| | | GTLTVEAPLPKAVTQSAEITIPVTFEARAQIGGPESEQSGAK |
| 17 | Mouse | MTERRVPFSLLRSPSWEPFRDWYPAHSRLFDQAFGVPRLPDEWSQWFSAAGWP |
| | HSP27 | GYVRPLPAATAEGPAAVTLAAPAFSRALNRQLSSGVSEIRQTADRWRVSLDVNH |
| | | FAPEELTVKTKEGVVEITGKHEERQDEHGYISRCFTRKYTLPPGVDPTLVSSSLSP |
| | | EGTLTVEAPLPKAVTQSAEITIPVTFEARAQIGGPEAGKSEQSGAK |
| 18 | Human | MDIAIHHPWIRRPFFPFHSPSRLFDQFFGEHLLESDLFPTSTSLSPFYLRPPSFLRAP |
| | CRYAB | SWFDTGLSEMRLEKDRFSVNLDVKHFSPEELKVKVLGDVIEVHGKHEERQDEH |
| | | GFISREFHRKYRIPADVDPLTITSSLSSDGVLTVNGPRKQVSGPERTIPITREEKPA |
| | | VTAAPKK |
| 19 | Bovine | MDIAIHHPWIRRPFFPFHSPSRLFDQFFGEHLLESDLFPASTSLSPFYLRPPSFLRAP |
| | CRYAB | SWIDTGLSEMRLEKDRFSVNLDVKHFSPEELKVKVLGDVIEVHGKHEERQDEHG |
| | | FISREFHRKYRIPADVDPLAITSSLSSDGVLTVNGPRKQASGPERTIPITREEKPAV |
| | | TAAPKK |
| 20 | Rat | MDIAIHHPWIRRPFFPFHSPSRLFDQFFGEHLLESDLFSTATSLSPFYLRPPSFLRAP |
| | CRYAB | SWIDTGLSEMRMEKDRFSVNLDVKHFSPEELKVKVLGDVIEVHGKHEERQDEH |
| | | GFISREFHRKYRIPADVDPLTITSSLSSDGVLTVNGPRKQASGPERTIPITREEKPA |
| | | VTAAPKK |
| 21 | Mouse | MDIAIHHPWIRRPFFPFHSPSRLFDQFFGEHLLESDLFSTATSLSPFYLRPPSFLRAP |
| | CRYAB | SWIDTGLSEMRLEKDRFSVNLDVKHFSPEELKVKVLGDVIEVHGKHEERQDEHG |
| | | FISREFHRKYRIPADVDPLTITSSLSSDGVLTVNGPRKQVSGPERTIPITREEKPAV |
| | | AAAPKK |
| 22 | Chicken | MDITIHNPLIRRPLFSWLTPSRIFDQIFGEHLQESELLPTSPSLSPFLMRSPFFRMPS |
| | CRYAB | WLETGLSEMRLEKDKFSVNLDVKHFSPEELKVKVLGDMIEIHGKHEERQDEHGF |
| | | IAREFSRKYRIPADVDPLTITSSLSLDGVLTVSAPRKQSDVPERSIPITREEKPAIAG |
| | | SQRK |
| 23 | Rabbit | MDIAIHHPWIRRPFFPFHSPSRLFDQFFGEHLLESDLFPTSTSLSPFYLRPPSFLRAP |
| | CRYAB | SWIDTGLSEMRLEKDRFSVNLDVKHFSPEELKVKVLGDVIEVHGKHEERQDEHG |
| | | FISREFHRKYRIPADVDPLTITSSLSSDGVLTVNGPRKQAPGPERTIPITREEKPAVT |
| | | AAPKK |
| 24 | Pig | MRRRLRSEVRPQQSQRDPSSCRRRARLSEYWKLHKTAYIRGWLELQLKELTGQL |
| | CRYAB | TLYIHPAAMDIAIHHPWIRRPFFPFHSPSRLFDQFFGEHLLESDLFPASTSLSPFYFR |
| | | PPSFLRAPSWIDTGLSEMRLEKDRFSVNLDVKHFSPEELKVKVLGDVIEVHGKHE |
| | | ERQDEHGFISREFHRKYRIPADVDPLTITSSLSSDGVLTVNGPRRQASGPERTIPIT |
| | | REEKPAVTAAPKK |
| 25 | Sheep | MDIAIHHPWIRRPFFPFHSPSRLFDQFFGEHLLESDLFPASTSLSPFYLRPPSFLRAP |
| | CRYAB | SWIDTGLSEVRLEKDRFSVNLDVKHFSPEELKVKVLGDVIEVHGKHEERQDEHG |
| | | FISREFHRKYRIPADVDPLTITSSLSSDGVLTMNGPRKQASGPERTIPITREEKPAV |
| | | TAAPKK |
| 26 | Human | MDVTIQHPWFKRTLGPFYPSRLFDQFFGEGLFEYDLLPFLSSTISPYYRQSLFRTV |
| | CRYAA | LDSGISEVRSDRDKFVIFLDVKHFSPEDLTVKVQDDFVEIHGKHNERQDDHGYIS |
| | | REFHRRYRLPSNVDQSALSCSLSADGMLTFCGPKIQTGLDATHAERAIPVSREEK |
| | | PTSAPSS |
| 27 | Rat | MDVTIQHPWFKRALGPFYPSRLFDQFFGEGLFEYDLLPFLSSTISPYYRQSLFRTV |
| | CRYAA | LDSGISELMTHMWFVMHQPHAGNPKNNPGKVRSDRDKFVIFLDVKHFSPEDLT |
| | | VKVLEDFVEIHGKHNERQDDHGYISREFHRRYRLPSNVDQSALSCSLSADGMLT |
| | | FSGPKVQSGLDAGHSERAIPVSREEKPSSAPSS |
| 28 | Bovine | MDIAIQHPWFKRTLGPFYPSRLFDQFFGEGLFEYDLLPFLSSTISPYYRQSLFRTVL |
| | CRYAA | DSGISEVRSDRDKFVIFLDVKHFSPEDLTVKVQEDFVEIHGKHNERQDDHGYISR |
| | | EFHRRYRLPSNVDQSALSCSLSADGMLTFSGPKIPSGVDAGHSERAIPVSREEKPS |
| | | SAPSS |
| 29 | Mouse | MDVTIQHPWFKRALGPFYPSRLFDQFFGEGLFEYDLLPFLSSTISPYYRQSLFRTV |
| | CRYAA | LDSGISELMTHMWFVMHQPHAGNPKNNPVKVRSDRDKFVIFLDVKHFSPEDLT |
| | | VKVLEDFVEIHGKHNERQDDHGYISREFHRRYRLPSNVDQSALSCSLSADGMLT |
| | | FSGPKVQSGLDAGHSERAIPVSREEKPSSAPSS |
| 30 | Chicken | MDITIQHPWFKRALGPLIPSRLFDQFFGEGLLEYDLLPLFSSTISPYYRQSLFRSVL |
| | CRYAA | ESGISEVRSDRDKFTIMLDVKHFSPEDLSVKIIDDFVEIHGKHSERQDDHGYISREF |
| | | HRRYRLPANVDQSAITCSLSSDGMLTFSGPKVPSNMDPSHSERPIPVSREEKPTSA |
| | | PSS |
| 31 | Dog | MDIAIQHPWFKRALGPFYPSRLFDQFFGEGLFEYDLLPFLSSTISPYYRQSLFRTVL |
| | CRYAA | DSGISEVRSDRDKFVIFLDVKHFSPEDLTVKVLEDFVEIHGKHNERQDDHGYISR |
| | | EFHRRYRLPSNVDQSALSCSLSADGMLTFSGPKVPSGVDAGHSERAIPVSREEKP |
| | | SSAPSS |
| 32 | Cat | MDIAIQHPWFKRALGPFYPSRLFDQFFGEGLFEYDLLPFLSSTISPYYRQSLFRTVL |
| | CRYAA | DSGISEVRSDRDKFVIFLDVKHFSPEDLTVKVLEDFVEIHGKHNERQDDHGYISR |
| | | EFHRRYRLPSNVDQSALSCSLSADGMLTFSGPKVPSGVDAGHSERAIPVSREEKP |
| | | SSAPSS |
| 33 | PIG | MDIAIQHPWFKRALGPFYPSRLFDQFFGEGLFEYDLLPFLSSTISPYYRQSLFRTVL |
| | CRYAA | DSGVSEVRSDRDKFVIFLDVKHFSPEDLTVKVQEDFVEIHGKHNERQDDHGYISR |
| | | EFHRRYRLPSNVDQSALSCSLSADGMLTFSGPKVPSGVDAGHSERAIPVSREEKP |
| | | SSAPTS |
| 34 | Human | MADGQMPFSCHYPSRLRRDPFRDSPLSSRLLDDGFGMDPFPDDLTASWPDWALP |
| | HSPB8 | RLSSAWPGTLRSGMVPRGPTATARFGVPAEGRTPPPFPGEPWKVCVNVHSFKPE |
| | | ELMVKTKDGYVEVSGKHEEKQQEGGIVSKNFTKKIQLPAEVDPVTVFASLSPEG |
| | | LLIIEAPQVPPYSTFGESSFNNELPQDSQEVTCT |
| 35 | E. Coli | MRNFDLSPLMRQWIGFDKLANALQNAGESQSFPPYNIEKSDDNHYRITLALAGF |
| | sHSP | RQEDLEIQLEGTRLSVKGTPEQPKEEKKWLHQGLMNQPFSLSFTLAENMEVSGA |
| | IbpB | TFVNGLLHIDLIRNEPEPIAAQRIAISERPALNS |
| 36 | Yeast | MSFNSPFFDFFDNINNEVDAFNRLLGEGGLRGYAPRRQLANTPAKDSTGKEVAR |
| | HSP26 | PNNYAGALYDPRDETLDDWFDNDLSLFPSGFGFPRSVAVPVDILDHDNNYELKV |
| | | VVPGVKSKKDIDIEYHQNKNQILVSGEIPSTLNEESKDKVKVKESSSGKFKRVITL |
| | | PDYPGVDADNIKADYANGVLTLTVPKLKPQKDGKNHVKKIEVSSQESWGN |
| 37 | Human | MSHRTSSTFRAERSFHSSSSSSSSSTSSSASRALPAQDPPMEKALSMFSDDFGSFM |
| | HSPB7 | RPHSEPLAFPARPGGAGNIKTLGDAYEFAVDVRDFSPEDIIVTTSNNHIEVRAEKL |
| | | AADGTVMNTFAHKCQLPEDVDPTSVTSALREDGSLTIRARRHPHTEHVQQTFRT |
| | | EIKI |
| 38 | Rat | MEIRVPVQPSWLRRASAPLPGFSTPGRLFDQRFGEGLLEAELASLCPAAIAPYYLR |
| | HSPB6 | APSVALPTAQVPTDPGYFSVLLDVKHFSPEEISVKVVGDHVEVHARHEERPDEH |
| | | GFIAREFHRRYRLPPGVDPAAVTSALSPEGVLSIQATPASAQASLPSPPAAK |
| 39 | ACD | DRFSVNLDVKHFSPEELKVK |
| 40 | Mycobac- | MATTLPVQRHPRSLFPEFSELFAAFPSFAGLRPTFDTRLMRLEDEMKEGRYEVRA |
| | terium | ELPGVDPDKDVDIMVRDGQLTIKAERIEQKDFDGRSEFAYGSFVRTVSLPVGAD |
| | tuberculo- | EDDIKATYDKGILTVSVAVSEGKPTEKHIQIRSTN |
| | sis Acr1 | |
| 41 | Mycobact | MNNLALWSRPVWDVEPWDRWLRDFFGPAATTDWYRPVAGDFTPAAEIVKDGD |
| | erium | DAVVRLELPGIDVDKDVNVELDPGQPVSRLVIRGEHRDEHTQDAGDKDGRTLR |
| | tuberculo | EIRYGSFRRSFRLPAHVTSEAIAASYDAGVLTVRVAGAYKAPAETQAQRIAITK |
| | sis Acr2 | |
| 42 | Human | MAGQAFRKFLPLFDRVLVERSAAETVTKGGIMLPEKSQGKVLQATVVAVGSGS |
| | HSP10 | KGKGGEIQPVSVKVGDKVLLPEYGGTKVVLDDKDYFLFRDGDILGKYVD |
| 43 | Human | MADGQMPFSCHYPSRLRRDPFRDSPLSSRLLDDGFGMDPFPDDLTASWPDWALP |
| | HSPB8 | RLSSAWPGTLRSGMVPRGPTATARFGVPAEGRTPPPFPGEPWKVCVNVHSFKPE |
| | | ELMVKTKDGYVEVSGKHEEKQQEGGIVSKNFTKKIQLPAEVDPVTVFASLSPEG |
| | | LLIIEAPQVPPYSTFGESSFNNELPQDSQEVTCT |
| 44 | Strongylo- | MNDRWMTPFVRDPLSVCPLGYGGPANLFNEMNMLERKMMNSLNMVDRNLTN |
| | ides ratti | NMELMEPCPEVVNNDKEFRVKMDVSHYGPNELKVTVRDNYLQVEGKHEEKTD |
| | HSP17.1 | KYGTIQRSFVRKYALPKGLTEENVKSELTKDGVLTVGGNKMAIEDKNVKTVPIE |
| | | YRK |
| 45 | Loa loa | MSLFRYNPRDYFYTSPMERFIVNLLDNTFDDRSYRPLQSVAPYWLHQPILNECNI |
| | HSP | GNALGEVLDEKDKFGVQVDVSHFHPKELSVSVRDRELTIEGHHKERTDQSGHGS |
| | | IERHFVRKYVMPEEVQPDTIESHLSDKGVLTICAAKTTVGTPAARNIPIRASPKEP |
| | | EAGDKSTSNSTEQSK |
| 46 | Human | DRFSVNLDVKHFSPEELKVK |
| | CRYAB | |
| | peptide7 | |
| | 3-92 | |
| 47 | Human | WIRRPFFPF |
| | CRYAB | |
| | peptide | |
| | 9-17 | |
| 48 | Human | EKDRFSVNLDVKHFS |
| | CRYAB | |
| | peptide | |
| | 71-85 | |
| 49 | Human | IFLDVKHFSPEDLTVKVQDD |
| | CRYAA | |
| | peptide7 | |
| | 3-92 | |
| 50 | Human | FVIFLDVKHFSPEDL |
| | CRYAA | |
| | peptide | |
| | 71-85 | |
| 51 | Human | YSRALSRQLSSGVSEIRHTA |
| | HSP27 | |
| | peptide7 | |
| | 3-92 | |
| 52 | Human | PAYSRALSRQLSSGV |
| | HSP27 | |
| | peptide | |
| | 71-85 | |

The art describes uses of CRYAB as a therapeutic agent, e.g., for treating an inflammatory disease. However, in compositions and methods of the present disclosure, CRYAB is delivered in connection with a therapeutic agent to provide immunotolerance or otherwise reduce, inhibit, or prevent an immune reaction to the therapeutic agent (including its packaging component). Thus, the dosage of CRYAB effective in providing immunotolerance to the therapeutic agent (including its packaging component), is less than the dosage of CRYAB effective in acting as a therapeutic agent. In the present methods and compositions, CRYAB is not intended to be a species of therapeutic agent.

### Therapeutic Agent

The compositions and methods include a therapeutic agent. In embodiments, the therapeutic agent comprises a nucleic acid, a peptide, a protein, a compound, a chemotherapeutic, a cell, or any combination thereof.

In embodiments, the therapeutic agent, when administered in the absence of a heat shock protein or functional fragment thereof, generates an immune response, such as an immune response in a subject. In contrast, the inclusion of at least one heat shock protein or functional fragment thereof, reduces, inhibits or prevents an immune response and/or confers immune tolerance to the therapeutic agent.

A therapeutic agent comprises an active agent, such as a nucleic acid, a peptide, a protein, a compound, a chemotherapeutic, a cell, or any combination thereof. In embodiments, a therapeutic agent includes cellular materials used in cell-based therapies, nucleic acid-based therapies, including but not limited to DNA and RNA-based therapeutics, as well as delivery of nucleic acids providing regulatory sequences, and nucleic acid editing sequences and protein-based tools (e.g., nucleases such as CAS-type nucleases), protein-based therapeutics, including polypeptides, proteins, antibodies and fragments thereof, and nucleic acids encoding protein-based therapeutics, to obtain a desired pharmacologic and/or physiologic effect. The effects could include: (a) preventing the disease from occurring in a subject which may be predisposed to the disease but has not yet been diagnosed as having it; (b) inhibiting the disease, i.e., arresting its development; or (c) relieving the disease, i.e., causing regression of the disease.

A therapeutic agent can optionally include a delivery vehicle, e.g., a packaging component, for the active agent. The packaging component may be a virus particle, a non-viral particle, a polymer coating or a molecule co-administered with the active agent. A therapeutic agent also can include an active agent without a packaging component. In some cases, a therapeutic agent can induce an immune response in a subject and such response can be directed to the therapeutic agent or a portion thereof. An immune response can be directed to the packaging component (or a portion or component thereof), to the active agent or a portion thereof, or to a combination of the packaging component and the active agent. In some cases, a therapeutic agent comprises an active agent and is administered without a packaging component, and in such cases an immune response can be directed to the active agent or a portion thereof.

In embodiments, a therapeutic agent useful in a herein disclosed method and composition may be presently undergoing regulatory approval and/or clinical development. Alternately, a therapeutic agent may have received regulatory approval and/or undergone clinical development.

The therapeutic agent may have previously received regulatory approval but was withdrawn from the market due to complications, e.g., due to unwanted immune responses in patients.

### Nucleic Acid Therapeutic Agents

In compositions and methods of the present disclosure, a therapeutic agent comprises a therapeutically effective amount of an RNA. The RNA comprises a small interfering RNA (siRNA), a microRNA (miRNA), a small hairpin RNA (shRNA), a messenger RNA (mRNA), an anti-sense nucleic acid (asRNA), and/or a guide RNA (gRNA).

In embodiments, the siRNA, miRNA, shRNA, asRNA, mRNA, and/or gRNA is a synthetic RNA. Any non-natural RNA of the present disclosure may be understood to be a “synthetic RNA”.

A synthetic RNA may be transcribed using any method (or kit) known in the art. For example, a commercially-available kit or components thereof may be used to synthesize RNA. In one example, a DNA template may be transcribed using the T7 High Yield RNA Synthesis Kit (New England Biolabs, Inc.), according to the manufacturer's instructions. Synthetic RNA can be diluted with nuclease-free water and an RNase inhibitor (e.g., Superase⋅In, Life Technologies Corporation) may be added.

In embodiments, the synthetic RNA may comprise one or more non-canonical nucleotides. In embodiments, the one or more non-canonical nucleotides avoids substantial cellular toxicity. Examples of non-canonical nucleotides include one or more of 5-hydroxycytidine, 5-methylcytidine, 5-hydroxyme thylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine, optionally at an amount of at least 50%, or at least 60%, or at least 70%, or at least 80%, or at least 90%, or 100% of the non-canonical nucleotides. A synthetic RNA may include one type of non-canonical nucleotide to replace a specific nucleotide, e.g., all cytidines may be replaced with 5-methylcytidine. Alternately, a synthetic RNA may include a mix of natural nucleotides and non-canonical nucleotides; the non-canonical nucleotides may be of one type or more than one type, e.g., only 5-methylcytidine and a mixture of 5-methylcytidine and 5-hydroxymethylcytidine. A synthetic RNA may have non-canonical nucleotides replacing other natural nucleotides. For example, a synthetic RNA may have all or some of its uridines replaced with non-canonical uridine residues and all or some of its cytidines replaced with non-canonical cytidines residues.

In embodiments, the synthetic RNA may comprise a 5′ cap structure. In any of the herein-disclosed embodiments and aspects, the synthetic RNA may comprise a Kozak consensus sequence. The synthetic RNA may comprise or further comprise a 5′-UTR which comprises a sequence that increases RNA stability in vivo, and the 5′-UTR optionally comprises an alpha-globin or beta-globin 5′-UTR. The synthetic RNA may comprise or further comprise a 3′-UTR which comprises a sequence that increases RNA stability in vivo, and the 3′-UTR optionally comprises an alpha-globin or beta-globin 3′-UTR. The synthetic RNA may comprise or further comprise a 3′ poly(A) tail. These additions to a synthetic RNA may be included in the DNA sequence encoding the RNA. Where appropriate, these additions may be added using any method (or kit) known in the art. For example, a commercially-available kit or components thereof may be used.

RNA interference (RNAi) can be useful for reducing the expression level of a target gene, e.g., in a method of gene therapy. As provided herein, the compositions and methods can include use of small hairpin RNA (shRNA) for suppressing expression of the target gene in a mammal. shRNA molecules are believed to direct sequence-specific degradation of mRNA in cells of various types after first undergoing processing by an RNase III enzyme called DICER into smaller dsRNA molecules comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. shRNAs can include RNA strands containing two complementary elements that hybridize to one another to form a stem, a loop, and optionally an overhang, e.g., a 3′ overhang. The stem can be approximately 19 bp long, the loop about 1-20, e.g., about 4-10, and about 6-8 nt long, and/or the overhang about 1-20, e.g., about 2-15 nt long. In certain cases, the stem can be minimally 19 nucleotides in length and can be up to approximately 29 nucleotides in length.

RNAi, useful for reducing the expression level of target mRNA, can be mediated by short interfering RNAs (siRNA), which typically comprise a double-stranded region approximately 19 nucleotides in length with 1-2 nucleotide 3′ overhangs on each strand, resulting in a total length of between approximately 21 and 23 nucleotides. An siRNA can comprise two RNA strands hybridized together, or can alternatively, comprise a single RNA strand that includes a self-hybridizing portion. siRNAs can include one or more free strand ends, which can include phosphate and/or hydroxyl groups. siRNAs typically can include a portion that hybridizes under stringent conditions with a target transcript. One strand of the siRNA (or, the self-hybridizing portion of the siRNA) can be precisely complementary with a region of the target transcript (e.g., a target mRNA transcript), meaning that the siRNA hybridizes to the target transcript without a single mismatch. In certain cases, perfect complementarity is not achieved. In some cases, the mismatches are located at or near the siRNA termini. siRNAs as provided herein can trigger degradation of mRNAs to which they are targeted (e.g., a target mRNA transcript), thereby also reducing the rate of protein synthesis.

In some cases, certain microRNAs (miRNAs), which bind to the 3′ UTR of an mRNA transcript can inhibit expression of a protein encoded by the template transcript by a mechanism related to but distinct from classic RNA interference, e.g., by reducing translation of the transcript rather than decreasing its stability. MicroRNAs can be between approximately 20 and 26 nucleotides in length, e.g., 22 nt in length. MicroRNAs can be used to destabilize target transcripts and/or block their translation (e.g., expression of the target gene).

In embodiments, a nucleic acid containing a DNA sequence encoding a desired siRNA sequence is delivered into a target cell via transfection or virally-mediated infection. Once in the cell, the DNA sequence is continuously transcribed into RNA molecules that loop back on themselves and form hairpin structures through intramolecular base pairing. These hairpin structures, once processed by the cell, are equivalent to transfected siRNA molecules and are used by the cell to mediate RNAi of the desired protein. The use of shRNA has an advantage over siRNA transfection as the former can lead to stable, long-term inhibition of protein expression. Inhibition of protein expression by transfected siRNAs is a transient phenomenon that does not occur for time periods longer than several days. In some cases, this can be preferable and desired. In cases where longer periods of protein inhibition are necessary, shRNA-mediated inhibition is preferable.

Antisense nucleic acids (e.g., DNA, RNA, i.e., asRNA, modified DNA, or modified RNA) are generally single-stranded nucleic acids complementary to a portion of a target nucleic acid (e.g., a target mRNA transcript) and, therefore, can bind to the target to form a duplex. Antisense nucleic acids can pair with a target mRNA to render the RNA a substrate for cleavage by the intranuclear enzyme RNase H. In some cases, antisense nucleic acids can mediate target mRNA degradation for extended period, e.g., weeks, months, or years. As provided herein, antisense nucleic acids that can be used in the compositions and methods provided herein are typically oligonucleotides that range from 15 to 35 nucleotides in length but can range from 10 up to approximately 50 nucleotides in length. Binding can reduce or inhibit the function of the target nucleic acid. For example, antisense nucleic acids can block transcription when bound to genomic DNA (e.g., the target gene), inhibit translation when bound to mRNA (e.g., an mRNA transcript), and/or lead to degradation of the nucleic acid. Reduction in expression of target genes can be achieved by the administration of antisense nucleic acids or peptide nucleic acids comprising sequences complementary to those of the mRNA that encodes the gene's polypeptide. Antisense technology and its applications are well known in the art.

Messenger RNA (mRNA) is a large family of RNA molecules that convey genetic information from DNA to the ribosome, where they specify the amino acid sequence of the protein products of gene expression. The RNA polymerase enzyme transcribes genes into primary transcript mRNA (known as pre-mRNA) leading to processed, mature mRNA. This mature mRNA is then translated into a polymer of amino acids: a protein, as summarized in the central dogma of molecular biology.

In methods and compositions of the present disclosure, mRNA can be useful for increasing the expression level of a target gene, e.g., as a variation of gene therapy. In this embodiment, a target gene is defective (e.g., contains a mutation which reduces protein expression or produces a mis-functioning protein) and the therapeutic agent in an mRNA that encodes for the protein that should have been expressed by the target gene.

In other embodiments, a therapeutic benefit is obtained when a protein is overexpressed.

In embodiments, the mRNA encodes a gene editing protein.

### Gene Editing Proteins

In compositions and methods of the present disclosure, a therapeutic agent comprises a therapeutically effective amount of a gene-editing protein or a nucleic acid encoding a gene-editing protein. The gene-editing protein recognizes, binds to, and/or creates a single- or double-stranded break in a gene's DNA sequence and reduces transcription of the gene. The gene-editing protein may be a CRISPR-associated protein 9 (Cas9), a Transcription Activator-Like Effector Nucleases (TALEN), or a Zinc Finger Nuclease (ZFN). Use of such gene-editing proteins may be considered a gene therapy.

By “gene-editing protein” is meant a protein that can, either alone or in combination with one or more other molecules, alter the DNA sequence of a cell, by way of non-limiting example, a nuclease, a TALEN, ZFN, a meganuclease, a nickase, a clustered regularly interspaced short palindromic repeat (CRISPR)-associated protein, a DNA-repair protein, a DNA-modification protein, a base-modification protein, a DNA methyltransferase, a protein that causes DNA demethylation, an enzyme for which DNA is a substrate or a natural or engineered variant, family-member, orthologue, domain, fragment or fusion construct thereof. The gene-editing protein may be modified by adding one or more Fc regions, PEGylation, and/or by additions that increase the protein's half-life.

Several naturally-occurring proteins contain DNA-binding domains that can recognize specific DNA sequences. For example, zinc fingers (ZFs) and transcription activator-like effectors (TALEs).

ZFNs are artificial restriction enzymes generated by fusing a zinc finger DNA-binding domain to a DNA-cleavage domain. Zinc finger domains can be engineered to target desired DNA sequences, e.g., a target gene, and this enables zinc-finger nucleases to target unique sequences within complex genomes. By taking advantage of endogenous DNA repair machinery, these reagents can be used to alter the genomes of higher organisms. ZFNs may be used in methods for inactivating genes.

TALEN are restriction enzymes that can be engineered to cut specific sequences of DNA. They are made by fusing a TAL effector DNA-binding domain to a DNA cleavage domain (a nuclease which cuts DNA strands). TALEs can be engineered to bind to practically any desired DNA sequence, e.g., a target gene, so when combined with a nuclease, DNA can be cut at specific locations. TALENs can be introduced into cells, for use in gene-editing.

Fusion proteins containing one or more of these ZFN or TALE DNA-binding domains and the cleavage domain of Fokl, Stsl, Stsl-HA, Stsl-HA2, Stsl-UHA, Stsl-UHA2, Stsl-HF, or Stsl-UHF endonuclease can be used to create a single- or double-strand break in a desired region of DNA in a cell.

Other gene-editing proteins include clustered regularly interspaced short palindromic repeat (CRISPR)-associated proteins. Cas9 (or “CRISPR-associated protein 9”) is an enzyme that uses CRISPR sequences as a guide to recognize and cleave specific strands of DNA that are complementary to the CRISPR sequence. Cas9 enzymes together with CRISPR sequences form the basis of a technology known as CRISPR-Cas9 that can be used to edit genes within organisms. Other CRISPR-associated proteins, including xCas9, Cas12a (Cpf1), Cas13a, Cas14, CasX, CasY, a Class 1 Cas protein, a Class 2 Cas protein, and MAD7, may be used to edit genes.

When an in vitro translated Cas9 protein is included in a composition, the composition may further comprise a guide RNA (gRNA) that recognizes and binds to a target gene. Alternately, a first composition may comprise an in vitro translated Cas9 protein and a second composition may comprise a gRNA that recognizes and binds to the target gene.

When a nucleic acid that encodes cas9 is included in a composition, the composition may further comprise a guide RNA (gRNA) that recognizes and binds to a target gene. In embodiments, when a nucleic acid that encodes cas9 is included in a composition, the composition may further comprise a second nucleic acid encoding a gRNA that recognizes and binds to the target gene. Alternately, a nucleic acid may encode cas9 and encode a gRNA that recognizes and binds to the target gene.

### Peptide and Protein-Based Therapeutic Agents

In compositions and methods of the present disclosure, a therapeutic agent comprises a therapeutically effective amount of a peptide or a protein.

Classes of protein therapeutic agents useful in the present disclosure include, but are not limited to, antibodies, peptides/proteins comprising antigen binding fragments, antibody-based drugs (e.g., antibody-drug conjugates (ADC), Fc fusion proteins, anticoagulants, blood factors, bone morphogenetic proteins, engineered protein scaffolds, enzymes, growth factors, hormones, interferons, interleukins, and thrombolytics.

Non-limiting examples of protein therapeutic agents useful in the present invention include Agalsidase alfa, Agalsidase beta, Alglucosidase alfa, Alpha-galactosidase, Chromogranin A, GDP-L-fucose synthase, Glucagon Like Peptide-1, glucose-6-phosphatase catalytic subunit-related protein, Glutamic acid decarboxylase 65, Granulocyte-colony stimulating factor, Granulocyte-macrophage colony-stimulating factor, Human islet amyloid polypeptide precursor protein, Human Plasma-derived Factor IX, Human Plasma-derived Factor VIII, Human Plasma-derived Factor VIII and Von Willebrand factor, IFNα, IFNβ, Insulin, Islet-specific glucose-6-phosphatase catalytic subunit related protein, Laronidase, Myelin oligodendrocyte glycoprotein, Preproinsulin, Proteolipid protein, Pseudomonas Exotoxin PE38, Recombinant Factor IX, Recombinant Factor VIII, Recombinant Interferon β-1a, Recombinant Interferon β-1b, Recombinant myelin basic protein, ß-glucocerebrosidase, Tyrosine phosphatase like protein, Tyrosine phosphatase-related islet antigen 2, Zinc Transporter 8, and Zinc transporter ZnT8, or therapeutically-effective peptide fragments thereof.

Antibody-based therapies, which may comprise administering an antibody (or a protein comprising antigen binding fragments) and/or administering an antibody-drug conjugate (ADC), often produce unwanted immune responses, e.g., directed to the antigen-binding component itself.

In embodiments, a subject may have disease or disorder characterized by a deficit of a specific protein. This deficit may be due to a gene mutation or an epigenetic cause in which the protein is insufficiently expressed. In embodiments, the therapeutic agent comprises the protein that is insufficiently expressed.

In embodiments, a subject may have a disease and disorder that would benefit from an overabundance of a specific protein. In embodiments, the therapeutic agent comprises the specific protein that provides the benefit.

Examples of proteins comprising antigen binding fragments include a single-chain antibody (scFv); a recombinant camelid heavy-chain-only antibody (WH); a shark heavy-chain-only antibody (VNAR); a microprotein; a darpin; an anticalin; an adnectin; an aptamer; an Fv; an Fab; an Fab; and an F(ab′)2; and an antibody or antigen binding domain thereof from an IgA (including subclasses IgA1 and IgA2), IgD, IgE, IgG (including subclasses IgG1, IgG2, IgG3, and IgG4), or IgM Fc domain, optionally a human Fc domain, or a hybrid and/or variant thereof.

There are numerous commercially-available therapeutic agents comprising antigen binding fragments which may be used in methods and compositions of the present invention. Examples include 3f8, 8h9, Abagovomab, Abciximab (REOPRO), Abituzumab, Abrezekimab, Abrilumab, Actoxumab, Adalimumab (HUMIRA amjevita), Adecatumumab, Ado-Trastuzumab Emtansine, Ado-Trastuzumab Emtansine (KADCYLA), Aducanumab, Afasevikumab, Afelimomab, Alacizumab pegol, Alefacept (AMEVIVE), Alemtuzumab, Alemtuzumab (CAMPATH), Alirocumab (PRALUENT), Alpelisib (PIQRAY), Altumomab pentetate, Amatuximab, Anatumomab mafenatox, Andecaliximab, Anetumab ravtansine, Anifrolumab, Anrukinzumab (IMA-638), Apolizumab, Aprutumab ixadotin, Arcitumomab, Ascrinvacumab, Aselizumab, Atezolizumab (TECENTRIQ), Atidortoxumab, Atinumab, Atorolimumab, Avelumab (BAVENCIO), Axicabtagene Ciloleucel (YESCARTA), Azintuxizumab vedotin, Bapineuzumab, Basiliximab (SIMULECT), Bavituximab, Bcd-100, Bectumomab, Begelomab, Belantamab mafodotin, Belimumab (BENLYSTA), Bemarituzumab, Benralizumab, Berlimatoxumab, Bermekimab, Bersanlimab, Bertilimumab, Besilesomab, Bevacizumab (AVASTIN), Bezlotoxumab (ZINPLAVA), Biciromab, Bimagrumab, Bimekizumab, Birtamimab, Bivatuzumab mertansine, Bleselumab, Blinatumomab (BLINCYTO), Blontuvetmab, Blosozumab, Bococizumab, Brazikumab, Brentuximab Vedotin (ADCETRIS), Briakinumab, Brodalumab (SILIQ), Brolucizumab, Brontictuzumab, Burosumab, Cabiralizumab, Camidanlumab tesirine, Camrelizumab, Canakinumab (ILARIS), Cantuzumab mertansine, Cantuzumab ravtansine, Caplacizumab, Caplacizumab-yhdp (CABLIVI), Capromab pendetide, Carlumab, Carotuximab, Catumaxomab, Cbr96-doxorubicin immunoconjugate, Cedelizumab, Cemiplimab, Cemiplimab-rwlc (LIBTAYO), Cergutuzumab amunaleukin, Certolizumab pegol (CIMZIA), Cetrelimab, Cetuximab (ERBITUX), Cibisatamab, Cirmtuzumab, Citatuzumab bogatox, Cixutumumab, Claudiximab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Cr6261, Crenezumab, Crizanlizumab, Crotedumab, Cusatuzumab, Dacetuzumab, Daclizumab (ZINBRYTA, ZENAPAX), Dalotuzumab, Dapirolizumab pegol, Daratumumab (DARZALEX), Dectrekumab, Demcizumab, Denintuzumab mafodotin, Denosumab (PROLIA, XGEVA), Depatuxizumab mafodotin, Derlotuximab biotin, Detumomab, Dezamizumab, Dinutuximab (UNITUXIN), Diridavumab, Domagrozumab, Dorlimomab aritox, Dostarlimab, Drozitumab, Ds-8201, Duligotuzumab, Dupilumab, Durvalumab (IMFINZI), Dusigitumab, Duvortuxizumab, Ecromeximab, Eculizumab (SOURIS), Edobacomab, Edrecolomab, Efalizumab (RAPTIVA), Efungumab, Eldelumab, Elezanumab, Elgemtumab, Elotuzumab (EMPLICITI), Elsilimomab, Emactuzumab, Emapalumab-lzsg (GAMIFANT), Emibetuzumab, Emicizumab, Enapotamab vedotin, Enavatuzumab, Enfortumab vedotin, Enlimomab pegol, Enoblituzumab, Enokizumab, Enoticumab, Ensituximab, Epitumomab cituxetan, Epratuzumab, Eptinezumab, Erenumab, Erlizumab, Ertumaxomab, Etaracizumab, Etigilimab, Etrolizumab, Evinacumab, Evolocumab (REPATHA), Exbivirumab, Fanolesomab, Faralimomab, Faricimab, Farletuzumab, Fasinumab, Fbta05, Felvizumab, Fezakinumab, Fibatuzumab, Ficlatuzumab, Figitumumab, Firivumab, Flanvotumab, Fletikumab, Flotetuzumab, Folfiri-Bevacizumab, Folfiri-Cetuximab, Fontolizumab, Foralumab, Foravirumab, Fremanezumab, Fresolimumab, Frovocimab, Frunevetmab, Fulranumab, Futuximab, Galcanezumab, Galiximab, Gancotamab, Ganitumab, Gantenerumab, Gatipotuzumab, Gavilimomab, Gedivumab, Gemtuzumab Ozogamicin (MYLOTARG), Gevokizumab, Gilvetmab, Gimsilumab, Girentuximab, Glembatumumab vedotin, Golimumab (SIMPONI, SIMPONI ARIA), Gomiliximab, Gosuranemab, Guselkumab, Ianalumab, Ibalizumab, Ibi308, Ibritumomab Tiuxetan (ZEVALIN), Icrucumab, Idarucizumab (PRAXBIND), Ifabotuzumab, Igovomab, Iladatuzumab vedotin, Imalumab, Imaprelimab, Imciromab, Imgatuzumab, Inclacumab, Indatuximab ravtansine, Indusatumab vedotin, Inebilizumab, Inflectra (REMICADE), Infliximab (REMICADE), Infliximab-dyyb (INFLEC IRA), Inolimomab, Inotuzumab Ozogamicin (BESPONSA), Intetumumab, Iomab-b, Ipilimumab (YERVOY), Iratumumab, Isatuximab, Iscalimab, Istiratumab, Itolizumab, Ixekizumab (TALTZ), Keliximab, Labetuzumab, Lacnotuzumab, Ladiratuzumab vedotin, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Lilotomab satetraxetan, Lintuzumab, Lirilumab, Lodelcizumab, Lokivetmab, Loncastuximab tesirine, Lorvotuzumab mertansine, Losatuxizumab vedotin, Lucatumumab, Lulizumab pegol, Lumiliximab, Lumretuzumab, Lupartumab amadotin, Lutikizumab, Mapatumumab, Margetuximab, Marstacimab, Maslimomab, Matuzumab, Mavrilimumab, Mepolizumab (NUCALA), Metelimumab, Milatuzumab, Minretumomab, Mirikizumab, Mirvetuximab soravtansine, Mitumomab, Modotuximab, Mogamulizumab, Mogamulizumab-kpkc (POTELIGEO), Monalizumab, Morolimumab, Mosunetuzumab, Motavizumab, Moxetumomab pasudotox, Moxetumomab Pasudotox-tdfk (LUMOXITI), Muromonab-cd3, Nacolomab tafenatox, Namilumab, Naptumomab estafenatox, Naratuximab emtansine, Narnatumab, Natalizumab (TYSABRI), Navicixizumab, Navivumab, Naxitamab, Nebacumab, Necitumumab (PORTRAZZA), Nemolizumab, Neod001, Nerelimomab, Nesvacumab, Netakimab, Nimotuzumab, Nirsevimab, Nivolumab, Nivolumab (OPDIVO), Nofetumomab merpentan, Obiltoxaximab (ANTHIM), Obinutuzumab (GAZYVA), Ocaratuzumab, Ocrelizumab, Odulimomab, Ofatumumab (ARZERRA), Olaratumab (LARTRUVO), Oleclumab, Olendalizumab, Olokizumab, Omalizumab (XOLAIR), Omburtamab, Oms721, Onartuzumab, Ontuxizumab, Onvatilimab, Opdivo (NIVOLUMAB), Opicinumab, Oportuzumab monatox, Oregovomab, Orticumab, Otelixizumab, Otilimab, Otlertuzumab, Oxelumab, Ozanezumab, Ozoralizumab, Pagibaximab, Palivizumab (SYNAGIS), Pamrevlumab, Panitumumab (VECTIBIX), Pankomab, Panobacumab, Parsatuzumab, Pascolizumab, Pasotuxizumab, Pateclizumab, Patritumab, Pdr001, PEG-Intron (Peginterferon Alfa-2b), Pembrolizumab (KEYTRUDA), Pemetrexed Disodium, Pemtumomab, Perakizumab, Pertuzumab (PERJETA), Pexelizumab, Pidilizumab, Pinatuzumab vedotin, Pintumomab, Placulumab, Plerixafor, Plozalizumab, Pogalizumab, Polatuzumab vedotin, Polatuzumab Vedotin-piiq (POLIVY), Ponezumab, Porgaviximab, Prasinezumab, Prezalizumab, Priliximab, Pritoxaximab, Pritumumab, Pro 140, Quilizumab, Racotumomab, Radretumab, Rafivirumab, Ralpancizumab, Ramucirumab, Ramucirumab (CYRAMZA), Ranevetmab, Ranibizumab (LUCENTIS), Ravagalimab, Ravulizumab, Ravulizumab-cwvz (ULTOMIRIS), Raxibacumab, Refanezumab, Regavirumab, Regn-eb3, Relatlimab, Remtolumab, Reslizumab (CINQAIR), Rilotumumab, Rinucumab, Risankizumab, Rituximab (RITUXAN), Rituximab (TRUXIMA), Rituximab and Hyaluronidase Human (RITUXAN HYCELA), Rivabazumab pegol, Rmab, Robatumumab, Roledumab, Romilkimab, Romosozumab, Rontalizumab, Rosmantuzumab, Rovalpituzumab tesirine, Rovelizumab, Rozanolixizumab, Ruplizumab, Sa237, Sacituzumab govitecan, Samalizumab, Samrotamab vedotin, Sarilumab, Satralizumab, Satumomab pendetide, Secukinumab (COSENTYX), Selicrelumab, Seribantumab, Setoxaximab, Setrusumab, Sevirumab, Sgn-cd19a, Shp647, Sibrotuzumab, Sifalimumab, Siltuximab (SYLVANT), Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Talacotuzumab, Talizumab, Tamtuvetmab, Tanezumab, Taplitumomab paptox, Tarextumab, Tavolimab, Tefibazumab, Telimomab aritox, Telisotuzumab vedotin, Tenatumomab, Teneliximab, Teplizumab, Tepoditamab, Teprotumumab, Tesidolumab, Tetulomab, Tezepelumab, Tgn1412, Tibulizumab, Tigatuzumab, Tildrakizumab, Timigutuzumab, Timolumab, Tiragotumab, Tislelizumab, Tisotumab vedotin, Tnx-650, Tocilizumab (ACTEMRA), Tomuzotuximab, Toralizumab, Tosatoxumab, Tositumomab, Tovetumab, Tralokinumab, Trastuzumab (HERCEPTIN), Trastuzumab and Hyaluronidase-oysk (HERCEPTIN HYLECTA), Trastuzumab emtansine, Trbs07, Tregalizumab, Tremelimumab, Trevogrumab, Tucotuzumab celmoleukin, Tuvirumab, Ublituximab, Ulocuplumab, Urelumab, Urtoxazumab, Ustekinumab (STELARA), Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Vunakizumab, Xentuzumab, Xmab-5574, Zalutumumab, Zanolimumab, Zatuximab, Zenocutuzumab, Ziralimumab, Zolbetuximab (IMAB362), and Zolimomab aritox.

A fragment, e.g., comprising the protein-binding domain, of an aforementioned protein may be used in a method or composition of the present disclosure. Moreover, the fragment may be included in a fusion protein which further comprises peptide domains that enhance stability and longevity of the fusion protein when compared to the fragment alone. In embodiments, two or more fragments may be combined to form a bi-functional/bi-valent fusion protein.

### Biologics

In embodiments, a therapeutic agent is a biologic drug. A biologic is a therapeutic product that is produced from living organisms or contain components of living organisms. Non-limiting examples of biologics that may be used in methods and compositions of the present invention include abatacept (Orencia), abobotulinumtoxinA (Dysport), aflibercept (Eylea), agalsidase beta (Fabrazyme), albiglutide (Tanzeum), aldesleukin (Proleukin), alglucosidase alfa (Myozyme, Lumizyme), alteplase (Cathflo Activase, Activase), anakinra (Kineret), asfotase alfa (Strensiq), asparaginase (Elspar), asparaginase Erwinia chrysanthemi (Erwinaze), becaplermin (Regranex), belatacept (Nulojix), collagenase (Santyl), collagenase Clostridium histolyticum (Xiaflex), dulaglutide (Trulicity), ecallantide (Kalbitor), elosulfase alfa (Vimizim), epoetin alfa (Epogen/Procrit), etanercept (Enbrel), etanercept-szzs (Erelzi), follitropin alpha (Gonal f), galsulfase (Naglazyme), glucarpidase (Voraxaze), iaronidase (Aldurazyme), idursulfase (Elaprase), incobotulinumtoxinA (Xeomin), interferon alfa-2b (Intron A), interferon alfa-n3 (Alferon N Injection), interferon beta-1a (Avonex), interferon beta-1a (Rebif), interferon beta-1b (Betaseron), interferon beta-1b (Extavia), interferon gamma-1b (Actimmune), methoxy polyethylene glycol-epoetin beta (Mircera), metreleptin (Myalept), ocriplasmin (Jetrea), onabotulinumtoxinA (Botox), oprelvekin (Neumega), palifermin (Kepivance), parathyroid hormone (Natpara), pegaptanib (Macugen), pegaspargase (Oncaspar), pegfilgrastim (Neulasta), peginterferon alfa-2a (Pegasys), peginterferon alfa-2b (Peglntron, Sylatron), peginterferon beta-1a (Plegridy), pegloticase (Krystexxa), rasburicase (Elitek), reteplase (Retavase), Rilonacept (Arcalyst), rimabotulinumtoxinB (Myobloc), romiplostim (Nplate), sargramostim (Leukine), sebelipase alfa (Kanuma), tenecteplase (TNKase), and ziv-aflibercept (Zaltrap).

### Chemotherapeutics

In embodiments, a therapeutic agent is a chemotherapeutic. Examples of chemotherapeutics include Actemra (Tocilizumab), Adcetris (Brentuximab Vedotin), Ado-Trastuzumab Emtansine, Alemtuzumab, Arzerra (Ofatumumab), Atezolizumab, Avastin (Bevacizumab), Avelumab, Bavencio (Avelumab), Besponsa (Inotuzumab Ozogamicin), Bevacizumab, Blinatumomab, Blincyto (Blinatumomab), Brentuximab Vedotin, Cablivi (Caplacizumab-yhdp), Campath (Alemtuzumab), Caplacizumab-yhdp, Cemiplimab-rwlc, Cetuximab, Cyramza (Ramucirumab), Daratumumab, Darzalex (Daratumumab), Denosumab, Dinutuximab, Durvalumab, Emapalumab-lzsg, Empliciti (Elotuzumab), Erbitux (Cetuximab), Folfiri-Bevacizumab, Folfiri-Cetuximab, Gamifant (Emapalumab-lzsg), Gazyva (Obinutuzumab), Gemtuzumab Ozogamicin, Herceptin Hylecta (Trastuzumab and Hyaluronidase-oysk), Herceptin (Trastuzumab), Ibritumomab Tiuxetan, Imfinzi (Durvalumab), Inotuzumab Ozogamicin, Ipilimumab, Kadcyla (Ado-Trastuzumab Emtansine), Keytruda (Pembrolizumab), Libtayo (Cemiplimab-rwlc), Lumoxiti (Moxetumomab Pasudotox-tdfk), Mogamulizumab-kpkc, Moxetumomab Pasudotox-tdfk, Mvasi (Bevacizumab), Mylotarg (Gemtuzumab Ozogamicin), Necitumumab, Nivolumab, Obinutuzumab, Ofatumumab, Opdivo (Nivolumab), Panitumumab, PEG-Intron (Peginterferon Alfa-2b), Pembrolizumab, Pemetrexed Disodium, PO eta (Pertuzumab), Pertuzumab, Piqray (Alpelisib), Plerixafor, Polatuzumab Vedotin-piiq, Polivy (Polatuzumab Vedotin-piiq), Portrazza (Necitumumab), Poteligeo (Mogamulizumab-kpkc), Prolia (Denosumab), Ramucirumab, Ravulizumab-cwvz, Rituxan (Rituximab), Rituxan Hycela (Rituximab and Hyaluronidase Human), Rituximab, Rituximab and Hyaluronidase Human, Siltuximab, Sylvant (Siltuximab), Tecentriq (Atezolizumab), Tocilizumab, Trastuzumab, Trastuzumab and Hyaluronidase-oysk, Truxima (Rituximab), Ultomiris (Ravulizumab-cwvz), Unituxin (Dinutuximab), Vectibix (Panitumumab), Xgeva (Denosumab), Yervoy (Ipilimumab), Yescarta (Axicabtagene Ciloleucel), and Zevalin (Ibritumomab Tiuxetan).

### Cell-Based Therapeutic Agents

In embodiments, a composition or method of the present disclosure comprises a cell-based therapeutic agent. Examples of cell-based therapeutic agents relate to Tumor-Infiltrating Lymphocyte (TIL) therapy, Engineered T Cell Receptor (TCR) therapy, Chimeric Antigen Receptor (CAR) T Cell therapy, Treg Cell therapy, CAR-Treg cell therapy, Dendritic Cell therapy, Natural Killer (NK) Cell therapy, and Stem-cell therapy. A cell used as a therapeutic agent may be allogenic or autogenic. The stem cell may have been reprogrammed from a somatic cell.

### Packaging Components

In some embodiments, a therapeutic agent comprises a packaging component that provides a means for containing, protecting, delivering, and/or stabilizing an active agent to a target cell, tissue or organ, such as in a subject.

In embodiments, the packaging component is a microcapsule, a particle, a viral vector, a virus, or a virus-like particle. In some cases, a packaging component or portion thereof can raise an immune response in a subject, and the administration of a heat shock protein as described herein can confer an immune tolerizing effect to such packaging component or portion thereof.

In embodiments, a therapeutic agent comprising a nucleic acid is DNA (e.g., plasmid DNA and linear DNA) or is RNA (e.g., mRNA, antisense RNA, miRNA, siRNA, and gRNA) that includes a packaging component. In embodiments, the therapeutic agent is administered as a nucleic acid encoding a peptide/protein-based therapeutic agents, a biologic, an antibody, or an antigen binding fragment, as disclosed herein, and includes a packaging component; the nucleic acid is transcribed and/or translated by a cell (e.g., in the subject). In embodiments, the therapeutic agent is administered as a nucleic acid encoding a peptide/protein-based therapeutic agent, a biologic, an antibody, or an antigen binding fragment as disclosed herein, and without a packaging component; the nucleic acid is transcribed and/or translated by a cell (e.g., in the subject).

In embodiments, a therapeutic agent comprises a viral gene therapy vector and such viral gene therapy vectors or portions thereof raise an immune response when administered to a subject.

One of the currently most effective and well understood vectors for use in gene therapy is the adeno-associated viruses (AAVs). Even though AAVs are generally thought to be only weakly immunogenic, it has been shown that AAVs can induce activation of dendritic cells (DCs). Several factors may influence the nature and severity of AAV induced immunogenicity. The organ or tissue type targeted for transduction may have an effect on the nature of the immune response to AAV treatment. In trials examining AAV mediated delivery of human clotting factor IX, it was found that transduction of liver tissue resulted in more stable expression of the transgene compared to transduction of skeletal muscle; this was subsequently determined to be a consequence of a lower immune response being triggered in hepatic delivery. Dosage seems to be another major determinant of the immunogenic response to AAV therapy. Higher doses of AAV particles have been shown to increase capsid specific T-cell activation, while lower doses are more susceptible to neutralization by pre-existing anti-AAV antibodies.

Inflammation at the site of delivery can also lead to negative outcomes with AAV therapy. For example, one study showed that IL-12 induced inflammation in the livers of mice concurrent with AAV. Similarly, treatment with pro-inflammatory molecules, such as LPS or CpG, or pro-inflammatory cytokines, such as IL-6 or TNFa, resulted in extinction of transgene expression in mice. The presence of ligands for various molecular pattern receptors, such as CpG sites in some AAV serotypes, may serve to promote inflammation in some cases. Without wishing to be bound by theory, these factors may combine/interact and contribute, in an additive fashion, to changes in the local cytokine environment, which will ultimately determine the nature of the immune response (immunogenic or tolerant) to the therapy.

Risk of an unwanted immune response may be greater when an AAV therapy is re-administered. Turnover of transfected cells results in the loss of transgene expression which can prompt re-administration of the AAV therapy to restore this lost expression. The re-administration increases the risk of an immune response, as antibody production by memory cells generated during the initial dose may be triggered upon redosing.

Methods to curb gene therapy-induced immunity and inflammation have been explored. However, these efforts in overcoming immunogenicity and promoting immunotolerance (the state of non-reactivity of the immune system to substances that have the capacity to induce an immune response) have not been successful. For example, T-cell depletion has been suggested as a method of enhancing the success of gene therapy techniques. However, T-cell depletion broadly suppresses immune activity leaving subjects at risk to opportunistic infections. One strategy being investigated is the use of empty vectors to “soak up” circulating anti-AAV antibodies. By adding a quantity of empty AAV capsids in doses of transgene carrying AAV vectors, it is possible to overcome antibody mediated inhibition to AAV therapy. However, increasing the overall dose of AAV runs the risk of triggering a cytotoxic T-cell response. This method also cannot prevent immunity developing in response to the transgene product.

In embodiments of the present disclosure overcoming immunogenicity and promoting immunotolerance of a viral gene-therapy is achieved by administration of a heat shock protein as described herein.

In embodiments, the viral gene therapy vector is an AAV vector. In embodiments, the viral gene therapy vector encapsulates one or more nucleic acids. In embodiments, the therapeutic agent is an rAAV vector. In embodiments, the therapeutic agent is rAAV vector encapsulating polynucleotides encoding multiple endocrine neoplasia type 1 and type 2 proteins (MEN-1 and MEN-2). In other particular embodiments, a therapeutic agent is rAAV vector encapsulating polynucleotides encoding hemophilia A or hemophilia B. In embodiments, other types of viral vectors not limiting to lentiviruses, adenoviruses, Herpes simplex viruses, and retroviruses can be utilized. In embodiments, therapeutic agents are used to treat Duchenne muscular dystrophy, Charcot-Marie Tooth Disease, Pompe's disease, other lysosomal storage diseases, ADA-SCID, and any other genetic diseases that are candidates for gene therapies.

In the context of viral vectors, a “therapeutic agent” is a viral vector comprising a nucleic acid. Alternately, the “therapeutic agent” is the nucleic acid that is packaged (e.g., contained) in a viral vector.

In some embodiments, a heat shock protein is co-formulated with a therapeutic agent such as viral vector.

In other embodiments, a heat shock protein is encoded by a nucleic acid that is packaged (e.g., contained) in a viral vector.

Many viral vectors useful in the herein disclosed methods and compositions, e.g., for gene therapy, have been described (see, e.g., Lundstrom, Trends Biotechnol., 21: 1 17, 122, 2003. Illustrative viral vectors include those selected from Antiviruses (LV), retroviruses (RV), adenoviruses (AV), adeno-associated viruses (AAV), and a viruses, although other viral vectors may also be used. In some in vivo uses, viral vectors that do not integrate into the host genome are suitable, such as a viruses and adenoviruses. Illustrative types of a viruses include Sindbis virus, Venezuelan equine encephalitis (VEE) virus, and Semliki Forest virus (SFV). For other uses, viral vectors that integrate into the host genome are suitable, such as retroviruses, AAV, and Antiviruses.

“AAV” is an abbreviation for adeno-associated virus, and may be used to refer to the virus itself or derivatives thereof. The term covers all subtypes and both naturally occurring and recombinant forms, except where required otherwise. The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), avian AAV, bovine AAV, canine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals. An “AAV virus” or “AAV viral particle” or “rAAV vector particle” refers to a viral particle composed of at least one AAV capsid protein (typically by all of the capsid proteins of a wild-type AAV) and an encapsulated polynucleotide rAAV vector. If the particle comprises a heterologous polynucleotide (i.e. a polynucleotide other than a wild-type AAV genome such as a transgene to be delivered to a mammalian cell), it is typically referred to as an “recombinant adeno-associated virus”, “rAAV vector particle” or simply an “rAAV vector”. Thus, production of rAAV particle necessarily includes production of rAAV vector, as such a vector is contained within an rAAV particle. Accordingly, the AAV may be a variant of a naturally-occurring AAV1, AAV2, AAV4, AAV5, AAV6, AAV7, AAV8, AAV9, AAV2/5, AAV2/2, AAV-DJ, or AAV-DJ8. By an AAV variant, is meant an AAV having a sequence identity of 70% or more to AAV1, AAV2, AAV4, AAV5, AAV6, AAV7, AAV8, AAV9, AAV2/5, AAV2/2, AAV-DJ, or AAV-DJ8, for example, a sequence identity of 80%, 85%, or 90% or more; of 91%, 92%, 93%, 94%, 95% or more, in some instances a sequence identity of 96%, 97%, 98%, or 99% to AAV1, AAV2, AAV4, AAV5, AAV6, AAV7, AAV8, AAV9, AAV2/5, AAV2/2, AAV-DJ, and AAV-DJ8.

In embodiments, the packaging component is a viral vector, a virus, or a virus-like particle. The viral vector may be a lentivirus; the viral vector may be an adeno-associated virus (AAV). Examples of AAV include AAV1, AAV2, AAV4, AAV5, AAV6, AAV7, AAV8, AAV9, AAV2/5, AAV2/2, AAV-DJ, and AAV-DJ8, and any combination thereof.

In embodiments, the AAV is AAV1, AAV5, AAV6, AAV8, or AAV9. AAVs are increasingly used for gene delivery in therapeutic applications because of their ability to transduce both dividing and non-dividing cells, their long-term persistence as episomal DNA in infected cells, and their low immunogenicity. These characteristics make them appealing for applications in gene therapy, including according to the therapeutic agents of the present disclosure.

Methods of producing and packaging AAVs are well known in the art. In embodiments, plasmid vectors are triple-transfected into mammalian cells (e.g., HEK293 cells) using standard transfection protocols. The first plasmid contains a transgene cassette (e.g., containing a nucleic acid which encodes or comprises an mRNA, shRNA, siRNA, miRNA, asRNA, and/or gRNA or containing a nucleic acid that encodes a gene-editing protein) flanked by inverted terminal repeat (ITR) sequences from a parental AAV virus. The transgene cassette has a promoter sequence and that drives transcription of a heterologous nucleic acid in the nucleus of a target cell. The second plasmid contains nucleic acids encoding an AAV Rep gene and a Cap gene. The third plasmid contains nucleic acids encoding helper virus proteins needed for viral assembly, and packaging of the heterologous nucleic acid into the modified capsid structure.

In embodiments, the packaging component is a microcapsule. The microcapsule may be a liposome, an albumin microsphere, a microemulsion, a nanoparticle (e.g., a lipid nanoparticle), and a nanocapsule and/or may comprise hydroxylmethylcellulose, gelatin-microcapsules, and/or polymethylmethacrylate.

In the context of microcapsule, a “therapeutic agent” is a microcapsule comprising a nucleic acid, a peptide, a protein, a compound, a chemotherapeutic, a cell, or any combination thereof. Alternately, the “therapeutic agent” is the nucleic acid, the peptide, the protein, the protein complex, the compound, the chemotherapeutic, the cell, or any combination thereof that is packaged (e.g., contained) in a microcapsule.

In embodiments, a heat shock protein is administered in a microcapsule, as described herein.

In embodiments, a nucleic acid (e.g., an mRNA or a plasmid DNA) encoding a heat shock protein is administered in a microcapsule, as described herein.

In embodiments, the microcapsule is a lipid nanoparticle or liposome.

By “lipid nanoparticle” or “liposome” is meant an entity containing amphiphilic molecules, hydrophobic molecules, or a mixture thereof, that is at least transiently stable in an aqueous environment, by way of non-limiting example, a micelle, a unilamellar bilayer with aqueous interior, a multilamellar bilayer, a lipid nanoparticle, any of the foregoing complexed with one or more nucleic acids, or a stable nucleic acid lipid particle.

Lipid nanoparticles and liposomes comprise one or more lipids and/or polymers that enhance uptake of their cargo (protein or nucleic acid) by cells. See, e.g., Prui et al., Crit Rev Ther Drug Carrier Syst., 2009; 26(6): 523-580; Wakasar, J Drug Target, 2018, 26(4):311-318, Langer, 1990, Science 249:1527-1533; Treat et al., in “Liposomes in the Therapy of Infectious Disease and Cancer”, Lopez-Berestein and Fidler (eds.), Liss, New York, pp. 353-365 (1989); the contents of each of which is incorporated herein by reference in its entirety.

In embodiments, microcapsules, e.g., lipid nanoparticles and liposomes, comprise lipids selected from one or more of the following categories: cationic lipids; anionic lipids; neutral lipids; multi-valent charged lipids; and zwitterionic lipids. In some cases, a cationic lipid may be used to facilitate a charge-charge interaction with nucleic acids and proteins or peptides contained therein.

In embodiments, a therapeutic agent and/or a heat shock protein, or a functional fragment thereof, included in a composition and/or method comprises a cationic liposome and/or cationic polymer formulation.

In embodiments, the microcapsule further comprises a PEGylated lipid.

In embodiments, a microcapsule comprises a Lipofectamine reagent (Life Technologies Corporation), e.g., Lipofectamine 2000, Lipofectamine 3000, Lipofectamine Stem, Lipofectamine RNAiMAX, and Invivofectamine 3.0.

Using Lipofectamine reagents as an example, a therapeutic agent or a heat shock protein (alternately, a nucleic acid encoding the same) and a Lipofectamine transfection reagent are diluted separately in a suitable complexation medium, mixed, and incubated together, according to the manufacturer's instructions.

Microcapsule preparations can be made according to standard protocols. For example, suitable lipids are diluted from stocks in ethanol to a desired concentration. A therapeutic agent or a heat shock protein (alternately, a nucleic acid encoding the same) is diluted to an appropriate concentration. Both solutions are transferred to syringes and mixed, e.g., using a Nanoassemblr Benchtop (Precision Nanosystems) as directed by the manufacturer. The resulting liposomes may then be formulated for in vitro or in vivo uses.

In embodiments, the heat shock protein and the therapeutic agent are provided in the same packaging component type. As examples, the heat shock protein is administered in a microcapsule and the therapeutic agent is also administered in a microcapsule or a nucleic acid encoding the heat shock protein is administered in a viral vector and the therapeutic agent is also administered in a viral vector.

In embodiments, the heat shock protein and the therapeutic agent are provided in different packaging component types. As examples, the heat shock protein is administered in a microcapsule and the therapeutic agent is administered in a viral vector or a nucleic acid encoding the heat shock protein is administered in a viral vector and the therapeutic agent is administered in a microcapsule.

In embodiments, the heat shock protein and/or the therapeutic agent are provided without a packaging component, e.g., in a pharmaceutical composition comprising an excipient but not a viral vector or microcapsule. As examples, the heat shock protein and the therapeutic agent are administered without a packaging component; a nucleic acid encoding the heat shock protein and the therapeutic agent are administered without a packaging component; the heat shock protein is administered without a packaging component and the therapeutic agent is administered in a viral vector; or a nucleic acid encoding the heat shock protein is administered without a packaging component and the therapeutic agent is administered in a liposome/lipid nanoparticle.

In embodiments, the immune tolerizing effect (provided by the heat shock protein or functional fragment thereof) is directed against the packaging component (e.g., a viral protein or a component of a microcapsule).

As used herein, a packaging component is not considered to be an excipient.

### Compositions

In embodiments, a composition disclosed herein is administered to a subject concurrently or during an overlapping time period with an additional at least one therapeutic agent. In other embodiments, the composition is administered first to the subject, after a time period has passed, then the additional at least one therapeutic agent is administered.

The compositions disclosed herein comprising at least one heat shock protein or a functional fragment thereof, induce immune tolerance and/or reduce, inhibit, or prevent an immune response of a subject to a therapeutic agent. Immune response and immune tolerance (collectively referred to as “immune effect”) can be monitored by any suitable method such as the qualitative or quantitative monitoring of biochemical, physical, and physiological markers and any combination thereof. In embodiments, immune effect is monitored by the visible symptoms of a subject. In embodiments, immune effect is monitored by measuring antibodies generated by the subject to the therapeutic agent after administration of at least one heat shock protein or a functional fragment and the therapeutic agent. In embodiments, immune effect is monitored by measuring or otherwise identifying the presence of antibodies (e.g., neutralizing antibodies) in a subject or in a biological sample from a subject. In embodiments, immune effect is monitored by measuring cytokine concentrations, for example, cytokine concentrations from monocyte-derived dendritic cells. In embodiments, immune effect is monitored by measuring induction of regulatory T-cells, such as by biomarkers, for example CD4, CD25, and FoxP3 or other available T cell markers.

The compositions of the present disclosure are formulated to be suitable for in vivo administration to a mammal. Such compositions can optionally comprise a suitable amount of a pharmaceutically acceptable excipient to provide the form for proper administration. In embodiments, acceptable excipients in the pharmaceutical compositions are preferably nontoxic to recipients at the dosages and concentrations employed. Pharmaceutical excipients can be liquids, such as water or saline. Acceptable excipients may include buffers such as phosphate, citrate, Ringer's, TBS, PBS, HEPES, HBSS, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, dried skim milk, and lysine, and carbohydrates such as starch, glucose, lactose, mannose, sucrose, sorbitol, glycerol, glycerol monostearate, and/or glycol. A pharmaceutical excipient may comprise sodium chloride, propylene, ethanol and the like. Pharmaceutical compositions of the disclosure may be administered locally or systemically using an injectable formulation. Pharmaceutical compositions for injection can be formulated using a sterile solution or any pharmaceutically acceptable liquid as a vehicle. Pharmaceutically acceptable vehicles comprise, but are not limited to, sterile water and physiological saline. In a preferred embodiment, a pharmaceutical composition provided herein is administered by injection or infusion.

Any composition described herein, if desired, can also comprise pH buffering agents.

The pharmaceutical composition can be in an acceptable diluent, or can comprise a slow release matrix in which the therapeutic agent and/or heat shock protein (with or without a packaging component) undergoes delayed release.

Dosage forms suitable for parenteral administration (e.g., intravenous injection or infusion, intraarterial injection or infusion, intra-lymphatic injection or infusion, intramuscular injection, intraperitoneal injection, subcutaneous injection, intra-dermal injection, epi-cutaneous injection, intra-peritoneal, and intra-arterial injection or infusion) or enteral administration (e.g., rectal) include, for example, solutions, suspensions, dispersions, emulsions, and the like.

Furthermore, pharmaceutical compositions disclosed herein may be formulated according to different methods of delivery. For examples, the pharmaceutical compositions can be formulated for inhalation administration, intratracheal administration, parenteral administration, subcutaneous administration, epi-cutaneous administration, intra-dermal administration, intravenous administration, intra-lymphatic administration, intramuscular administration, intra-arterial administration, intrathecal administration, intra-peritoneal administration, or intraperitoneal administration. The pharmaceutical composition may also be formulated for, or administered via, nasal, spray, oral, aerosol, rectal, or vaginal administration. Exemplary tissue targets may include liver, skeletal muscle, lung, vascular endothelium, epithelial, and/or hematopoietic cells.

In embodiments, a route of administering the therapeutic agent and/or the heat shock protein is selected from the group consisting of mucosal, intra-nasal, oral, intra-vaginal, pulmonary, transdermal, intra-venous, sublingual, intra-dermal, epi-cutaneous, intra-lymphatic, intra-peritoneal, rectal, and intra-muscular.

In another aspect, the present disclosure provides a pharmaceutical composition comprising an immune tolerizing effective amount of a heat shock protein. The immune tolerizing effective amount of the heat shock protein reduces and/or inhibits an immune response to a therapeutic agent when administered to a subject.

In embodiments, the immune tolerizing effective amount of a heat shock protein in a pharmaceutical composition increases an amount of antigen-specific regulatory T cells (Tregs) in the subject. The antigen-specific Tregs recognize the therapeutic agent or a portion thereof and/or the immune tolerizing effective amount of a heat shock protein increases an amount of an expression level of CD25 and/or FoxP3 on the antigen-specific Tregs.

In embodiments, the heat shock protein in a pharmaceutical composition is αB-crystallin (CRYAB), αA-crystallin (CRYAA), HSP60, HSP70, HSP72, HSP84, HSP90, HSP104, GP96, HSP33, HSP27, HSP22, HSP20, HSP12, HSP10, HSP7, or a functional fragment thereof.

In embodiments, the heat shock protein comprises a small heat shock protein (sHsp) or a functional fragment thereof. In embodiments, the sHsp comprises one or more features selected from (i) a subunit molecular mass between about 12 and about 43 kDa, (ii) an α-crystallin domain, (iii) an N-terminal domain and (iv) C-terminal extension.

In embodiments, the heat shock protein in a pharmaceutical composition is CRYAB or CRYAA.

In embodiments, the CRYAB in a pharmaceutical composition comprises a sequence selected from the group consisting of SEQ ID NO: 18-25. In embodiments, the CRYAB comprises a sequence of SEQ ID NO: 18. In embodiments, the CRYAB comprises a sequence that is at least 65%, 70%, 75%, 80%, 85%, 90%, 95%, 96%, 97%, 98%, 99% identical to SEQ ID NO:18. In embodiments, the functional fragment of CRYAB is selected from the group consisting of SEQ ID NO:46 to SEQ ID NO:48. In embodiments, the functional fragment of CRYAB is at least 50 amino acids in length, e.g., at least 5, 6, 7, 8, 9, 10, 11, 12, 13, 14, 15, 16, 17, 18, 19, 20, 21, 25, 30, 40, 50, 60, 70, 80, 90, 100, or 125, and any number amino acids therebetween.

In embodiments, the immune tolerizing effect is directed against a therapeutic agent and/or a packaging component of the therapeutic agent.

In embodiments, the composition further comprises a therapeutic agent which comprises a nucleic acid, a peptide, a protein, a compound, a chemotherapeutic, a cell, or any combination thereof. In embodiments, the therapeutic agent comprises a packaging component which is a microcapsule, a particle, a viral vector, a virus, or a virus-like particle.

In embodiments, the composition further comprises an immunosuppressant.

In another aspect, the present disclosure provides a pharmaceutical composition comprising an immune tolerizing effective amount of a heat shock protein for treating a cardiovascular, endocrine, gastrointestinal, genetic, hematologic, infectious, metabolic, neurological/psychiatric, oncological (e.g., cancer), ophthalmologic, respiratory, and/or urological disease or disorder.

### Formulations/Dosages

The dosage of any herein-disclosed composition (comprising a therapeutic agent and/or a heat shock protein) can depend on several factors including the characteristics of the mammal to be administered. Examples of characteristics include species, strain, sex, age, weight, health, and/or disease status. In addition, the dosage of the pharmaceutical compositions depends on factors including the route of administration and the disease to be treated. Dosage may be adjusted to provide the optimum therapeutic response. Typically, a dosage may be an amount that effectively treats a disease without inducing significant toxicity and/or inducing an undesirable immune response.

The doses of each of the therapeutic agent and the heat shock protein will depend on the specific therapeutic agent and heat shock protein.

The timing of doses (of either or both the therapeutic agent and the heat shock protein) will depend on the specific therapeutic agent and heat shock protein administered.

Dosages and timings of administrations can be determined on a case-by-case basis.

In some embodiments, a dosage or administration regimen is determined by administering to a subject a therapeutic agent and the subject's immune response is determined. A heat shock protein is then administered, and the subject's immune response is determined. The amount, frequency of dosing or multiplicity of doses of the therapeutic agent is increased until an effective amount of the therapeutic agent is reached with an acceptable safety profile. In some embodiments, the amount, frequency and/or number of doses of the heat shock protein is increased to maintain immune tolerance.

In embodiments, the effectiveness of the heat shock protein to induce immune tolerance is measured by detecting and quantifying serum markers representative of an immune response. Examples of serum markers representative of an immune response include C-reactive protein (CRP), erythrocyte sedimentation rate (ESR), and procalcitonin (PCT). Other markers include serum amyloid A, cytokines (e.g., interleukin-6 (IL-6), IL-35, IL-4, IL-10, IL-12, TGF-β, TNF-α, IFNγ, PTX3, TSG6/TNFAIP6, and CCL20), alpha-1-acid glycoprotein, plasma viscosity, ceruloplasmin, hepcidin, haptoglobin, IgM antibody levels, anti-antigen IgG levels, and IgG antibody levels.

In embodiments, am immune tolerizing effect comprises a reduction of the amount of an anti-therapeutic-agent antibody in the subject.

In embodiments, the effectiveness of the heat shock protein to induce immune tolerance is measured by detecting and quantifying a change in the quantity of immune-related cells overall or a change in a subset of immune-related cells, e.g., an increase in Treg cell populations and/or a reduction in T cytolytic cell populations) may be used to measure the effectiveness of the immune tolerizing agent to induce immune tolerance. Here, in vitro differentiation assays may be used with or without flow cytometry assays.

Other suitable assays, such as anti-antigen neutralizing antibody analysis, transgene level analysis, analysis of cytokine release, analysis of CD8/CD4 T cell responses, adoptive transfer experiments, antigen specificity experiments, immunohistochemistry, anti-AAV T and B cell ELISpot assays, and RT-PCR can be utilized.

Additionally, well-known symptoms of inflammation may be informative when determining dosage and/or timing of the therapeutic agent and/or the heat shock protein. Common symptoms of inflammation include, but are not limited to, fatigue, fever, mouth sores, rashes, abdominal pain, and chest pain.

In embodiments, the immune tolerizing effect comprises the modulation of the expression of one or more of IL-10, TGFbeta, IL-35, perforin, granzyme, Fas, Fas-L, galectin-1, galectin-9, TIM-3, TRAIL, PD-1, CTLA-4, PD-L1, SLAMF1, and LAG3.

In embodiments, the immune tolerizing effect comprises a change in the number and/or ratio regulatory T cells, Tr1 cells, and/or exhausted T cells (e.g., low IL-2, low proliferation, and low IFN-g T cells).

In embodiments, the immune tolerizing effect comprises disruption in the metabolic pathways in T effector cells (e.g. cAMP).

In embodiments, the immune tolerizing effect comprises modulation of antigen-presenting cell (e.g. dendritic cells) maturation and function as a consequence of CTLA4:CD80/86 interaction and upregulation of indoleamine 2,3-dioxygenase (IDO).

In embodiments, the method comprises administering to the subject one or more subsequent doses of the therapeutic agent, e.g., a gene therapy, wherein one or more subsequent doses is effective to generate a therapeutic response. In embodiments, the therapeutic response to the subsequent dose of the therapeutic agent is enhanced or improved as compared with the response when the heat shock protein is not administered; the administration of the heat shock protein reduces, prevents, or alleviates an immune response to the subsequent dose of the therapeutic agent; or the administration of the heat shock protein reduces, prevents, or alleviates inactivation of the subsequent dose of the therapeutic agent.

Generally, immune tolerizing effect of the heat shock proteins allows a higher dose of the therapeutic composition, more frequent dosing, and/or a longer duration of the treatment. Additionally, due to a reduction of an adverse immune response by the heat shock proteins, the overall method provides a greater therapeutic benefit than a regimen without the heat shock protein.

In embodiments, a method of the present disclosure, which includes heat shock proteins, allows administration of higher doses of the therapeutic agent to a subject in need thereof. For example, an undesirable immune response may result from a standard method when the therapeutic agent is administered at a typical dose; however, in a method of the present disclosure, which includes heat shock proteins, the therapeutic agent may be administered a higher dose without an undesirable immune response or with reduced amounts of an undesirable immune response. The higher dose is at least 5% greater than the standard dose for the therapeutic agent. As examples, the higher dose is at least 5% greater, 6% greater, 7% greater, 8% greater, 9% greater, 10% greater, 11% greater, 12% greater, 13% greater, 14% greater, 15% greater, 16% greater, 17% greater, 18% greater, 19% greater, 20% greater, 25% greater, 30% greater, 35% greater, 40% greater, 45% greater, 50% greater, 55% greater, 60% greater, 65% greater, 70% greater, 75% greater, 80% greater, 85% greater, 90% greater, 95% greater, 100% greater, 125% greater, 150% greater, 175% greater, 200% greater, 300% greater, 400% greater, 500% greater, or 1000% greater, and any value therebetween, than the typical dose for the therapeutic agent.

In embodiments, a method of the present disclosure, which includes heat shock proteins, allows administration of the therapeutic agent to a subject for a longer overall duration. For example, a standard method may continue for three months and need to be halted due to an undesirable immune response; however, a method of the present disclosure may continue for an additional month or a few extra months due to the absence of or reduction in undesirable immune response. The longer duration is at least 5% longer than the duration that is typical for the therapeutic agent. As examples, the longer duration is at least 5% longer, 6% longer, 7% longer, 8% longer, 9% longer, 10% longer, 11% longer, 12% longer, 13% longer, 14% longer, 15% longer, 16% longer, 17% longer, 18% longer, 19% longer, 20% longer, 25% longer, 30% longer, 35% longer, 40% longer, 45% longer, 50% longer, 55% longer, 60% longer, 65% longer, 70% longer, 75% longer, 80% longer, 85% longer, 90% longer, 95% longer, 100% longer, 125% longer, 150% longer, 175% longer, 200% longer, 300% longer, 400% longer, 500% longer, or 1000% longer, and any value therebetween, than the typical duration for the therapeutic agent.

In embodiments, a method of the present disclosure, which includes heat shock proteins, allows more frequent administration of the therapeutic agent. For example, an undesirable immune response may result from a standard method when the therapeutic agent is administered at the typical frequency (e.g., once per week); however, a method of the present disclosure may be administered a higher frequency (e.g., twice per week) without an undesirable immune response or with reduced amounts of an undesirable immune response. The higher frequency is at least 5% more frequent than the frequency that is typical for the therapeutic agent. As examples, the higher frequency is at least 5% higher, 6% higher, 7% higher, 8% higher, 9% higher, 10% higher, 11% higher, 12% higher, 13% higher, 14% higher, 15% higher, 16% higher, 17% higher, 18% higher, 19% higher, 20% higher, 25% higher, 30% higher, 35% higher, 40% higher, 45% higher, 50% higher, 55% higher, 60% higher, 65% higher, 70% higher, 75% higher, 80% higher, 85% higher, 90% higher, 95% higher, 100% higher, 125% higher, 150% higher, 175% higher, 200% higher, 300% higher, 400% higher, 500% higher, or 1000% higher, and any value therebetween, than the typical frequency for the therapeutic agent.

In embodiments, a method of the present disclosure, which includes heat shock proteins, results in a decrease in immune response relative to a method that lacks a heat shock protein. For example, a standard method may produce a quantifiable immune response (e.g., a defined increase in an immune-related serum marker and a particular change in the number/ratio of immune cell types) that is normalized to an untreated subject; however, a method of the present disclosure may produce a normalized decrease in the quantifiable immune response relative to a subject that received the therapeutic agent but not the heat shock protein. The normalized decrease in immune response is an at least 5% decrease. As examples, the decrease is at least a 5% decrease, a 6% decrease, a 7% decrease, a 8% decrease, a 9% decrease, a 10% decrease, a 11% decrease, a 12% decrease, a 13% decrease, a 14% decrease, a 15% decrease, a 16% decrease, a 17% decrease, a 18% decrease, a 19% decrease, a 20% decrease, a 25% decrease, a 30% decrease, a 35% decrease, a 40% decrease, a 45% decrease, a 50% decrease, a 55% decrease, a 60% decrease, a 65% decrease, a 70% decrease, a 75% decrease, a 80% decrease, a 85% decrease, a 90% decrease, a 95% decrease, a 100% decrease, a 125% decrease, a 150% decrease, a 175% decrease, a 200% decrease, a 300% decrease, a 400% decrease, a 500% decrease, or a 1000% decrease, and any value therebetween, relative to the normalized quantifiable immune response resulting from the therapeutic agent.

Additionally, the methods of the present disclosure, which includes heat shock proteins, are more effective (at a given dose, timing, and/or duration) than a method lacking a heat shock protein. Thus, a subject may be administered a composition comprising a therapeutic agent at a lower dosage or one that is understood to be effective if delivered without an immune tolerizing agent such as a heat shock protein. By administering a lower dosage, the cost in administering a therapeutic agent can be reduced. Moreover, the frequency of the dosing of the therapeutic agent may be reduced; thereby further reducing costs.

In embodiments, a subsequent administration (e.g., of a therapeutic agent relative to a heat shock protein, of a therapeutic agent relative to an immunosuppressant, or of a heat shock protein relative to an immunosuppressant, and vice versa) may be 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, 5 years, or 10 years after the immediately previous administration.

In embodiments, a prior administration (e.g., of a therapeutic agent relative to a heat shock protein, of a therapeutic agent relative to an immunosuppressant, or of a heat shock protein relative to an immunosuppressant, and vice versa) may be 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, 5 years, or 10 years before the immediately previous administration.

### Immunosuppressant Co-Therapy

In embodiments, a method of the present disclosure further comprises administration of an immunosuppressant.

In embodiments, the immunosuppressant is administered to the subject concurrently with the administering of the heat shock protein and concurrently with the administering of the therapeutic agent.

In embodiments, the immunosuppressant is administered to the subject concurrently with the administering of the heat shock protein and before the administering of the therapeutic agent.

In embodiments, the immunosuppressant is administered to the subject concurrently with the administering of the heat shock protein and after the administering of the therapeutic agent.

In embodiments, the immunosuppressant is administered to the subject before the administering of the heat shock protein and concurrently with the administering of the therapeutic agent.

In embodiments, the immunosuppressant is administered to the subject before the administering of the heat shock protein and before the administering of the therapeutic agent, e.g., the heat shock protein is before the therapeutic agent or vice versa.

In embodiments, the immunosuppressant is administered to the subject before the administering of the heat shock protein and after the administering of the therapeutic agent.

In embodiments, the immunosuppressant is administered to the subject after the administering of the heat shock protein and concurrently with the administering of the therapeutic agent.

In embodiments, the immunosuppressant is administered to the subject after the administering of the heat shock protein and before the administering of the therapeutic agent.

In embodiments, the immunosuppressant is administered to the subject after the administering of the heat shock protein and after the administering of the therapeutic agent, e.g., the heat shock protein is after the therapeutic agent or vice versa.

Non-limiting example of immunosuppressants include cyclosporine, prednisone, dexamethasone, hydrocortisone, methotrexate, azathioprine, mercaptopurine, dactinomycin, mycophenolate, mitomycin C, bleomycin, mithramycin, rapamycin, tacrolimus, deforolimus, everolimus, temsirolimus, zotarolimus, biolimus A9, pemecrolimus, voclosporin and sirolimus. Also, antibody therapeutics that modulate immune system may be used. Non-limiting examples include the use of an anti-TNFalpha antibody (e.g., adalimumab), an anti-CD20 antibody (e.g., rituximab), and anti-human thymocyte immunoglobulins (e.g., Thymoglobulin).

### Diseases

In embodiments, the subject treated by a method of the present disclosure has a disease or disorder selected from a cardiovascular, endocrine, gastrointestinal, genetic, hematologic, infectious, metabolic, neurological/psychiatric, oncological (e.g., cancer), ophthalmologic, respiratory, and/or urological disease or disorder.

In embodiments, the subject does not have a disease or disorder associated with inflammation. In embodiments, the methods and compositions of the present disclosure are not directed towards reducing inflammation associated with a disease.

In embodiments, the subject has a cardiovascular disease selected from the group consisting of Angina; Atherosclerosis; Cerebrovascular Accident (Stroke); Cerebrovascular disease; Congestive Heart Failure; Coronary Artery Disease; Myocardial infarction (Heart Attack); and Peripheral vascular disease.

In embodiments, the subject has an endocrine disease selected from the group consisting of Adrenal disorders; Glucose homeostasis disorders; Thyroid disorders; Calcium homeostasis disorders and Metabolic bone disease; Pituitary gland disorders; and Sex hormone disorders.

In embodiments, the subject has a gastrointestinal disease selected from Irritable Bowel Syndrome, biliary colic, renal colic, diarrhea-dominant irritable bowel syndrome, and pain associated with GI distension.

In embodiments, the subject has a genetic disorder selected from the group consisting of: 18p deletion syndrome, 1p36 deletion syndrome, 21-hydroxylase deficiency, AAA syndrome (achalasia-addisonianism-alacrima syndrome), Aarskog-Scott syndrome, ABCD syndrome, Aceruloplasminemia, Acheiropodia, Achondrogenesis type II, achondroplasia, Acute intermittent porphyria, adenylosuccinate lyase deficiency, Adrenoleukodystrophy, ADULT syndrome, Aicardi-Goutières syndrome, Alagille syndrome, Albinism, Alexander disease, alkaptonuria, Alpha 1-antitrypsin deficiency, Alport syndrome, Alstrom syndrome, Alternating hemiplegia of childhood, Alzheimer's disease, Amelogenesis imperfecta, Aminolevulinic acid dehydratase deficiency porphyria, Amyotrophic lateral sclerosis—Frontotemporal dementia, Androgen insensitivity syndrome, Angelman syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Birt-Hogg-Dubè syndrome, Björnstad syndrome, Bloom syndrome, Brody myopathy, Brunner syndrome, CADASIL syndrome, Campomelic dysplasia, Canavan disease, CARASIL syndrome, Carpenter Syndrome, Cerebral dysgenesis-neuropathy-ichthyosis-keratoderma syndrome (SEDNIK), Charcot-Marie-Tooth disease, CHARGE syndrome, Chédiak-Higashi syndrome, Chronic granulomatous disorder, Cleidocranial dysostosis, Cockayne syndrome, Coffin-Lowry syndrome, Cohen syndrome, collagenopathy, types II and XI, Color blindness, Congenital insensitivity to pain with anhidrosis (CIPA), Congenital Muscular Dystrophy, Cornelia de Lange syndrome (CDLS), Cowden syndrome, CPO deficiency (coproporphyria), Cranio-lenticulo-sutural dysplasia, Cri du chat syndrome, Crohn's disease, Crouzon syndrome, Crouzonodermoskeletal syndrome (Crouzon syndrome with acanthosis nigricans), Cystic fibrosis, Darier's disease, De Grouchy syndrome, Dent's disease (Genetic hypercalciuria), Denys-Drash syndrome, DiGeorge syndrome, Distal hereditary motor neuropathies, multiple types, Distal muscular dystrophy, Dravet syndrome, Duchenne muscular dystrophy, Edwards Syndrome, Ehlers-Danlos syndrome, Emery-Dreifuss syndrome, Epidermolysis bullosa, Erythropoietic protoporphyria, Fabry disease, Factor V Leiden thrombophilia, Familial adenomatous polyposis, Familial Creutzfeld-Jakob Disease, Familial dysautonomia, Familial hypercholesterolemia, Fanconi anemia (FA), Fatal familial insomnia, Feingold syndrome, FG syndrome, Fragile X syndrome, Friedreich's ataxia, G6PD deficiency, Galactosemia, Gaucher disease, Gerstmann-Sträussler-Scheinker syndrome, Gillespie syndrome, Glutaric aciduria, type I and type 2, GRACILE syndrome, Griscelli syndrome, Hailey-Hailey disease, Harlequin type ichthyosis, Hemochromatosis, hereditary, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary neuropathy with liability to pressure palsies (HNPP), Hereditary spastic paraplegia (infantile-onset ascending hereditary spastic paralysis), Hermansky-Pudlak syndrome, Heterotaxy, Homocystinuria, Hunter syndrome, Huntington's disease, Hurler syndrome, Hutchinson-Gilford progeria syndrome, Hyperlysinemia, Hyperoxaluria, primary, Hyperphenylalaninemia, Hypoalphalipoproteinemia (Tangier disease), Hypochondrogenesis, Hypochondroplasia, Immunodeficiency-centromeric instability-facial anomalies syndrome (ICF syndrome), Incontinentia pigmenti, Ischiopatellar dysplasia, Isodicentric 15, Jackson-Weiss syndrome, Joubert syndrome, Juvenile primary lateral sclerosis (JPLS), Keloid disorder, Kniest dysplasia, Kosaki overgrowth syndrome, Krabbe disease, Kufor-Rakeb syndrome, LCAT deficiency, Lesch-Nyhan syndrome, Li-Fraumeni syndrome, Limb-Girdle Muscular Dystrophy, lipoprotein lipase deficiency, Lynch syndrome, Malignant hyperthermia, Maple syrup urine disease, Marfan syndrome, Maroteaux-Lamy syndrome, McCune-Albright syndrome, McLeod syndrome, Mediterranean fever, familial, MEDNIK syndrome, Menkes disease, Methemoglobinemia, Methylmalonic acidemia, Micro syndrome, Microcephaly, Morquio syndrome, Mowat-Wilson syndrome, Muenke syndrome, Multiple endocrine neoplasia type 1 (Wermer's syndrome), Multiple endocrine neoplasia type 2, Muscular dystrophy, Muscular dystrophy, Duchenne and Becker type, Myostatin-related muscle hypertrophy, myotonic dystrophy, Natowicz syndrome, Neurofibromatosis, type I or type II, Niemann-Pick disease, Nonketotic hyperglycinemia, Nonsyndromic deafness, Noonan syndrome, Norman-Roberts syndrome, Ogden syndrome, Omenn syndrome, Osteogenesis imperfecta, Pantothenate kinase-associated neurodegeneration, PCC deficiency (propionic acidemia), Pendred syndrome, Peutz-Jeghers syndrome, Pfeiffer syndrome, Phenylketonuria, Pipecolic acidemia, Pitt-Hopkins syndrome, Polycystic kidney disease, Polycystic ovary syndrome (PCOS), Porphyria, Porphyria cutanea tarda (PCT), Prader-Willi syndrome, Primary ciliary dyskinesia (PCD), Primary pulmonary hypertension, Protein C deficiency, Protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, Shprintzen-Goldberg syndrome, Sickle cell disease, Siderius X-linked mental retardation syndrome, Sideroblastic anemia, Sjogren-Larsson syndrome, Sly syndrome, Smith-Lemli-Opitz syndrome, Smith-Magenis syndrome, Snyder-Robinson syndrome, Spinal muscular atrophy, Spinocerebellar ataxia (types 1-29), Spondyloepiphyseal dysplasia congenita (SED), SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thanatophoric dysplasia, Treacher Collins syndrome, Tuberous sclerosis complex (TSC), Turner syndrome, Usher syndrome, Variegate porphyria, von Hippel-Lindau disease, Waardenburg syndrome, Weissenbacher-Zweymüller syndrome, Williams syndrome, Wilson disease, Wolf-Hirschhorn syndrome, Woodhouse-Sakati syndrome, Xeroderma pigmentosum, X-linked intellectual disability and macroorchidism (fragile X syndrome), X-linked severe combined immunodeficiency (X-SCID), X-linked sideroblastic anemia (XLSA), X-linked spinal-bulbar muscle atrophy (spinal and bulbar muscular atrophy), Xp11.2 duplication syndrome, and Zellweger syndrome.

In embodiments, the subject has a hematological disease selected from acute myeloid leukemia, acute promyelocytic leukemia, acute lymphoblastic leukemia, chronic myelogenous leukemia, myelodysplastic syndromes, and sickle cell anemia.

In embodiments, the subject has an infectious disease selected from viral, bacterial or protozoological infectious diseases, selected from influenza, malaria, SARS, yellow fever, AIDS, Lyme borreliosis, Leishmaniasis, anthrax, meningitis, viral infectious diseases such as AIDS, Condyloma acuminata, hollow warts, Dengue fever, three-day fever, Ebola virus, cold, early summer meningoencephalitis (FSME), flu, shingles, hepatitis, herpes simplex type I, herpes simplex type II, Herpes zoster, influenza, Japanese encephalitis, Lassa fever, Marburg virus, measles, foot-and-mouth disease, mononucleosis, mumps, Norwalk virus infection, Pfeiffer's glandular fever, smallpox, polio (childhood lameness), pseudo-croup, fifth disease, rabies, warts, West Nile fever, chickenpox, cytomegalic virus (CMV), bacterial infectious diseases, anthrax, appendicitis, borreliosis, botulism, Campylobacter, Chlamydia trachomatis, cholera, diphtheria, donavanosis, epiglottitis, typhus fever, gas gangrene, gonorrhea, rabbit fever, Helicobacter pylori, whooping cough, climatic bubo, osteomyelitis, Legionnaire's disease, leprosy, listeriosis, pneumonia, meningitis, bacterial meningitis, anthrax, otitis media, Mycoplasma hominis, neonatal sepsis (Chorioamnionitis), noma, paratyphus, plague, Reiter's syndrome, Rocky Mountain spotted fever, Salmonella paratyphus, Salmonella typhus, scarlet fever, syphilis, tetanus, tripper, tsutsugamushi disease, tuberculosis, typhus, vaginitis (colpitis), soft chancre, and infectious diseases caused by parasites, protozoa or fungi, such as amoebiasis, bilharziosis, Chagas disease, Echinococcus, fish tapeworm, fish poisoning (Ciguatera), fox tapeworm, athlete's foot, canine tapeworm, candidosis, yeast fungus spots, scabies, cutaneous Leishmaniosis, lambliasis (giardiasis), lice, malaria, microscopy, onchocercosis (river blindness), fungal diseases, bovine tapeworm, schistosomiasis, sleeping sickness, porcine tapeworm, toxoplasmosis, trichomoniasis, trypanosomiasis (sleeping sickness), visceral Leishmaniosis, nappy/diaper dermatitis, miniature tapeworm, or prion diseases, including Creutzfeldt-Jakob disease, BSE, scrapie, and Kuru.

In embodiments, the subject has a metabolic disorder selected from the group consisting of type 2 diabetes mellitus, impaired glucose tolerance (IGT), impaired fasting blood glucose (IFG), hyperglycemia, postprandial hyperglycemia, overweight, obesity, metabolic syndrome and gestational diabetes.

In embodiments, the subject has a neurological/psychiatric disorder selected from the group consisting of Abarognosis; Acquired Epileptiform Aphasia; Acute disseminated encephalomyelitis; Adrenoleukodystrophy; Agenesis of the corpus callosum; Agnosia; Aicardi syndrome; Alexander disease; Alien hand syndrome; Allochiria; Alpers' disease; Alternating hemiplegia; Alzheimer's disease; Amyotrophic lateral sclerosis (see Motor Neuron Disease); Anencephaly; Angelman syndrome; Angiomatosis; Anoxia; Aphasia; Apraxia; Arachnoid cysts; Arachnoiditis; Amold-Chiari malformation; Arteriovenous malformation; Ataxia Telangiectasia; Attention deficit hyperactivity disorder; Auditory processing disorder; Autonomic Dysfunction; Back Pain; Batten disease; Behcet's disease; Bell's palsy; Benign Essential Blepharospasm; Benign Intracranial Hypertension; Bilateral frontoparietal polymicrogyria; Binswanger's disease; Blepharospasm; Bloch-Sulzberger syndrome; Brachial plexus injury; Brain abscess; Brain damage; Brain injury; Brain tumor; Brown-Sequard syndrome; Canavan disease; Carpal tunnel syndrome; Causalgia; Central pain syndrome; Central pontine myelinolysis; Centronuclear myopathy; Cephalic disorder; Cerebral aneurysm; Cerebral arteriosclerosis; Cerebral atrophy; Cerebral gigantism; Cerebral palsy; Cerebral vasculitis; Cervical spinal stenosis; Charcot-Marie-Tooth disease; Chiari malformation; Chorea; Chronic fatigue syndrome; Chronic pain; Coffin Lowry syndrome; Coma; Complex regional pain syndrome; Compression neuropathy; Congenital facial diplegia; Corticobasal degeneration; Cranial arteritis; Craniosynostosis; Creutzfeldt-Jakob disease; Cumulative trauma disorders; Cushing's syndrome; Cytomegalic inclusion body disease (CIBD); Cytomegalovirus Infection; Dandy-Walker syndrome; Dawson disease; De Morsier's syndrome; Dejerine-Klumpke palsy; Dejerine-Sottas disease; Delayed sleep phase syndrome; Dementia; Dermatomyositis; Developmental dyspraxia; Diabetic neuropathy; Diffuse sclerosis; Dravet syndrome; Dysautonomia; Dyscalculia; Dysgraphia; Dyslexia; Dystonia; Empty sella syndrome; Encephalitis; Encephalocele; Encephalotrigeminal angiomatosis; Encopresis; Epilepsy; Erb's palsy; Erythromelalgia; Essential tremor; Fabry's disease; Fahr's syndrome; Fainting; Familial spastic paralysis; Febrile seizures; Fisher syndrome; Friedreich's ataxia; Fibromyalgia; Gaucher's disease; Gerstmann's syndrome; Giant cell arteritis; Giant cell inclusion disease; Globoid Cell Leukodystrophy; Gray matter heterotopia; HTLV-1 associated myelopathy; Hallervorden-Spatz disease; Head injury; Headache; Hemifacial Spasm; Hereditary Spastic Paraplegia; Heredopathia atactica polyneuritiformis; Herpes zoster oticus; Herpes zoster; Hirayama syndrome; Holoprosencephaly; Huntington's disease; Hydranencephaly; Hydrocephalus; Hypercortisolism; Hypoxia; Inclusion body myositis; Incontinentia pigmenti; Infantile phytanic acid storage disease; Infantile Refsum disease; Infantile spasms; Intracranial cyst; Intracranial hypertension; Joubert syndrome; Karak syndrome; Keams-Sayre syndrome; Kennedy disease; Kinsbourne syndrome; Klippel Feil syndrome; Krabbe disease; Kugelberg-Welander disease; Kuru; Lafora disease; Lambert-Eaton myasthenic syndrome; Landau-Kleffner syndrome; Lateral medullary (Wallenberg) syndrome; Learning disabilities; Leigh's disease; Lennox-Gastaut syndrome; Lesch-Nyhan syndrome; Leukodystrophy; Lewy body dementia; Lissencephaly; Locked-In syndrome; Lou Gehrig's disease (See Motor Neurone Disease); Lumbar disc disease; Lumbar spinal stenosis; Lyme disease—Neurological Sequelae; Machado-Joseph disease (Spinocerebellar ataxia type 3); Macrencephaly; Macropsia; Megalencephaly; Melkersson-Rosenthal syndrome; Menieres disease; Meningitis; Menkes disease; Metachromatic leukodystrophy; Microcephaly; Micropsia; Migraine; Miller Fisher syndrome; Mini-stroke (transient ischemic attack); Mitochondrial myopathy; Mobius syndrome; Monomelic amyotrophy; Motor Neuron Disease; Motor skills disorder; Moyamoya disease; Mucopolysaccharidoses; Multi-infarct dementia; Multifocal motor neuropathy; Multiple sclerosis; Multiple system atrophy; Muscular dystrophy; Myalgic encephalomyelitis; Myasthenia gravis; Myelinoclastic diffuse sclerosis; Myoclonic Encephalopathy of infants; Myoclonus; Myopathy; Myotubular myopathy; Myotonia congenita; Narcolepsy; Neurofibromatosis; Neuroleptic malignant syndrome; Neurological manifestations of AIDS; Neurological sequelae of lupus; Neuromyotonia; Neuronal ceroid lipofuscinosis; Neuronal migration disorders; Niemann-Pick disease; Non 24-hour sleep-wake syndrome; Nonverbal learning disorder; O'Sullivan-McLeod syndrome; Occipital Neuralgia; Occult Spinal Dysraphism Sequence; Ohtahara syndrome; Olivopontocerebellar atrophy; Opsoclonus myoclonus syndrome; Optic neuritis; Orthostatic Hypotension; Overuse syndrome; Palinopsia; Paresthesia; Parkinson's disease; Paramyotonia Congenita; Paraneoplastic diseases; Paroxysmal attacks; Parry-Romberg syndrome; Pelizaeus-Merzbacher disease; Periodic Paralyses; Peripheral neuropathy; Persistent Vegetative State; Pervasive developmental disorders; Photic sneeze reflex; Phytanic acid storage disease; Pick's disease; Pinched nerve; Pituitary tumors; PMG; Polio; Polymicrogyria; Polymyositis; Porencephaly; Post-Polio syndrome; Postherpetic Neuralgia (PEN); Postinfectious Encephalomyelitis; Postural Hypotension; Prader-Willi syndrome; Primary Lateral Sclerosis; Prion diseases; Progressive hemifacial atrophy; Progressive multifocal leukoencephalopathy; Progressive Supranuclear Palsy; Pseudotumor cerebri; Rabies; Ramsay-Hunt syndrome (Type I and Type II); Rasmussen's encephalitis; Reflex neurovascular dystrophy; Refsum disease; Repetitive motion disorders; Repetitive stress injury; Restless legs syndrome; Retrovirus-associated myelopathy; Rett syndrome; Reye's syndrome; Rhythmic Movement Disorder; Romberg syndrome; Saint Vitus dance; Sandhoff disease; Schizophrenia; Schilder's disease; Schizencephaly; Sensory integration dysfunction; Septo-optic dysplasia; Shaken baby syndrome; Shingles; Shy-Drager syndrome; Sjogren's syndrome; Sleep apnea; Sleeping sickness; Snatiation; Sotos syndrome; Spasticity; Spina bifida; Spinal cord injury; Spinal cord tumors; Spinal muscular atrophy; Spinocerebellar ataxia; Steele-Richardson-Olszewski syndrome; Stiff-person syndrome; Stroke; Sturge-Weber syndrome; Subacute sclerosing panencephalitis; Subcortical arteriosclerotic encephalopathy; Superficial siderosis; Sydenham's chorea; Syncope; Synesthesia; Syringomyelia; Tarsal tunnel syndrome; Tardive dyskinesia; Tarlov cyst; Tay-Sachs disease; Temporal arteritis; Tetanus; Tethered spinal cord syndrome; Thomsen disease; Thoracic outlet syndrome; Tic Douloureux; Todd's paralysis; Tourette syndrome; Toxic encephalopathy; Transient ischemic attack; Transmissible spongiform encephalopathies; Transverse myelitis; Traumatic brain injury; Tremor; Trigeminal neuralgia; Tropical spastic paraparesis; Trypanosomiasis; Tuberous sclerosis; Von Hippel-Lindau disease; Viliuisk Encephalomyelitis; Wallenberg's syndrome; Werdnig-Hoffman disease; West syndrome; Whiplash; Williams syndrome; Wilson's disease; and Zellweger syndrome.

In embodiments, the subject has an oncological disease or disorder selected from melanomas, malignant melanomas, colon carcinomas, lymphomas, sarcomas, blastomas, renal carcinomas, gastrointestinal tumors, gliomas, prostate tumors, bladder cancer, rectal tumors, stomach cancer, esophageal cancer, pancreatic cancer, liver cancer, mammary carcinomas (breast cancer), uterine cancer, cervical cancer, acute myeloid leukemia (AML), acute lymphoid leukemia (ALL), chronic myeloid leukemia (CML), chronic lymphocytic leukemia (CLL), hepatomas, various virus-induced tumors such as, for example, papilloma virus-induced carcinomas (e.g. cervical carcinoma and cervical cancer), adenocarcinomas, herpes virus-induced tumors (e.g. Burkitt's lymphoma, EBV-induced ß-cell lymphoma), hepatitis ß-induced tumors (hepatocell carcinomas), HTLV-1- and HTLV-2-induced lymphomas, acoustic neuroma, lung carcinomas (e.g., lung cancer and bronchial carcinoma), small-cell lung carcinomas, pharyngeal cancer, anal carcinoma, glioblastoma, rectal carcinoma, astrocytoma, brain tumors, retinoblastoma, basalioma, brain metastases, medulloblastomas, vaginal cancer, pancreatic cancer, testicular cancer, Hodgkin's syndrome, meningiomas, Schneeberger disease, hypophysis tumor, Mycosis fungoides, carcinoids, neurinoma, spinalioma, Burkitt's lymphoma, laryngeal cancer, renal cancer, thymoma, corpus carcinoma, bone cancer, non-Hodgkin's lymphomas, urethral cancer, CUP syndrome, head/neck tumors, oligodendroglioma, vulval cancer, intestinal cancer, colon carcinoma, esophageal carcinoma (esophageal cancer), wart involvement, tumors of the small intestine, craniopharyngeomas, ovarian carcinoma, genital tumors, ovarian cancer (ovarian carcinoma), pancreatic carcinoma (pancreatic cancer), endometrial carcinoma, liver metastases, penile cancer, tongue cancer, gall bladder cancer, leukemia, plasmocytoma, lid tumor, and prostate cancer (prostate tumors).

In embodiments, the subject has an ophthalmologic disease selected from the group consisting of Disorders of eyelid, lacrimal system and orbit; Disorders of conjunctiva; Disorders of sclera, cornea, iris and ciliary body; Disorders of lens; Disorders of choroid and retina, Other disorders of choroid, Chorioretinal disorders in diseases classified elsewhere, Retinal detachments and breaks, Retinal vascular occlusions, other retinal disorders, Glaucoma; Disorders of vitreous body and globe; Disorders of optic nerve and visual pathways; Disorders of ocular muscles, binocular movement, accommodation and refraction; Visual disturbances and blindness; and Other disorders of eye and adnexa.

In embodiments, the subject has a respiratory disease selected from the group consisting of chronic obstructive pulmonary disease, asthma, pneumonia, hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, lung cancer, asbestosis, aspergilloma, aspergillosis, bronchiectasis, chronic bronchitis, emphysema, eosinophilic pneumonia, idiopathic pulmonary fibrosis, invasive pneumococcal disease, influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary histiocytosis X, pulmonary hypertension, pulmonary nocardiosis, pulmonary tuberculosis, pulmonary veno-occlusive disease, rheumatoid lung disease, sarcoidosis, and Wegener's granulomatosis. For example, the disease is chronic obstructive pulmonary disease (COPD). For example, the disease is asthma.

In embodiments, the subject has a urological disease selected from the group consisting of erectile dysfunction; impotence; premature ejaculation; female sexual dysfunction; female sexual arousal dysfunction; hypoactive sexual arousal disorder; vaginal atrophy; dyspaneuria; atrophic vaginitis; benign prostatic hyperplasia (BPH) or hypertrophy or enlargement; bladder outlet obstruction; bladder pain syndrome (BPS); interstitial cystitis (IC); overactive bladder, neurogenic bladder and incontinence; diabetic nephropathy;

The methods and compositions of present disclosure include any therapeutic agent useful for treating or reducing a symptom of a disease or disorder selected from a cardiovascular, endocrine, gastrointestinal, genetic, hematologic, infectious, metabolic, neurological/psychiatric, oncological (e.g., cancer), ophthalmologic, respiratory, and/or urological disease or disorder, as disclosed herein.

### Subjects

The term “subject” herein refers to a vertebrate, preferably a mammal, more preferably a human. The methods described herein can be useful in human therapeutics, veterinary applications, and/or preclinical studies in animal models of a disease or condition.

In embodiments, the subject is a mammal, e.g., a human, mouse, rat, guinea pig, dog, cat, horse, cow, pig, rabbit, sheep, or non-human primate, such as a monkey, chimpanzee, or baboon.

In embodiments, the subject is a non-human animal, and therefore the invention pertains to veterinary use. In a specific embodiment, the non-human animal is a companion animal, including a household pet. In another specific embodiment, the non-human animal is a livestock animal.

In embodiments, the mammal is a human.

In embodiments, the human is an adult human. In embodiments, the human has an age in a range of from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old, or older. In embodiments, the human is a juvenile. In embodiments, the human has an age in a range of from less than a year to about 10 years old, e.g., about 1 year old, about 2 years old, about 3 years old, about 4 years old, about 5 years old, about 6 years old, about 7 years old, about 8 years old, about 9 years old, and about 10 years old.

Several embodiments are described herein with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

### Definitions

The terminology used herein is for the purpose of describing particular cases only and is not intended to be limiting.

As used herein, the singular forms “a”, “an” and “the” are intended to include the plural forms as well, unless the context clearly indicates otherwise.

The terms “including”, “includes”, “having”, “has”, “with”, or variants thereof are used in either the detailed description and/or the claims, such terms are intended to be inclusive in a manner similar to the term “comprising”.

Ranges can be expressed herein as from “about” or “approximately” one particular value, and/or to “about” or “approximately” another particular value. When such a range is expressed, another case includes from the one particular value and/or to the other particular value. Similarly, when values are expressed as approximations, by use of the antecedent “about” or “approximately”, it will be understood that the particular value forms another case. It will be further understood that the endpoints of each of the ranges are significant both in relation to the other endpoint, and independently of the other endpoint. The term “about” or “approximately” as used herein refers to a range that is 15% plus or minus from a stated numerical value within the context of the particular usage. For example, about 10 would include a range from 8.5 to 11.5. The term “about” or “approximately” also accounts for typical error or imprecision in measurement of values.

The term “effective amount” or “therapeutically effective amount” refers to that amount of a composition described herein that is sufficient to affect the intended application, including but not limited to disease treatment, as defined below. The therapeutically effective amount may vary depending upon the intended treatment application (in a cell or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in a target cell. The specific dose will vary depending on the particular composition chosen, the dosing regimen to be followed, whether it is administered in combination with other compounds, timing of administration, the tissue to which it is administered, and the physical delivery system in which it is carried.

A “fragment” of a nucleotide or peptide sequence is meant to refer to a sequence that is less than that believed to be the “full-length” sequence.

A “functional fragment” of a DNA or protein sequence refers to a biologically active fragment of the sequence that is shorter than the full-length or reference DNA or protein sequence, but which retains at least one biological activity (either functional or structural) that is substantially similar to a biological activity of the full-length or reference DNA or protein sequence.

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

The terms “polypeptide,” “peptide,” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides such as anti-angiogenic polypeptides, neuroprotective polypeptides, and the like, when discussed in the context of delivering a gene product to a mammalian subject, and compositions therefor, refer to the respective intact polypeptide, or any fragment or genetically engineered derivative thereof, which retains the desired biochemical function of the intact protein. Similarly, references to nucleic acids encoding anti-angiogenic polypeptides, nucleic acids encoding neuroprotective polypeptides, and other such nucleic acids for use in delivery of a gene product to a mammalian subject (which may be referred to as “transgenes” to be delivered to a recipient cell), include polynucleotides encoding the intact polypeptide or any fragment or genetically engineered derivative possessing the desired biochemical function.

A “gene” refers to a polynucleotide containing at least one open reading frame that is capable of encoding a particular gene product after being transcribed, and sometimes also translated. The term “gene” or “coding sequence” refers to a nucleotide sequence in vitro or in vivo that encodes a gene product. In some instances, the gene consists or consists essentially of coding sequence, that is, sequence that encodes the gene product. In other instances, the gene comprises additional, non-coding, sequence. For example, the gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

The terms “composition” or “pharmaceutical composition” can refer to a biologically active compound, optionally mixed with at least one pharmaceutically acceptable chemical component, such as, though not limited to carriers, stabilizers, diluents, dispersing agents, suspending agents, thickening agents, excipients and the like.

The term “effective amount” or “therapeutically effective amount” refers to that amount of a composition described herein that is sufficient to affect the intended application, including but not limited to suppress immune response, suppress inflammatory cytokines, promote immunotolerance, as defined herein. The therapeutically effective amount may vary depending upon the intended treatment application (in a cell or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in a target cell. The specific dose will vary depending on the particular composition chosen, the dosing regimen to be followed, whether it is administered in combination with other compounds, timing of administration, the tissue to which it is administered, and the physical delivery system in which it is carried.

The terms “administer,” “administering”, “administration,” and the like, as used herein, can refer to the methods that are used to enable delivery of therapeutics or pharmaceutical compositions to a subject, and includes delivery systemically and locally to the desired site of biological action.

Any aspect or embodiment described herein can be combined with any other aspect or embodiment as disclosed herein.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Heat Shock Proteins Induce Immunotolerance

In this example, the anti-inflammatory effects of heat shock proteins on human dendritic cells (DCs) was characterized.

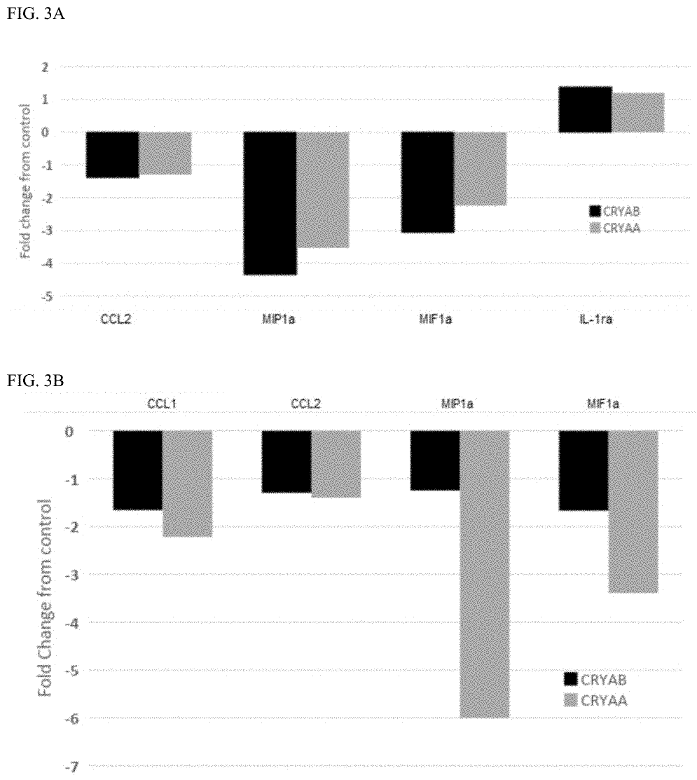

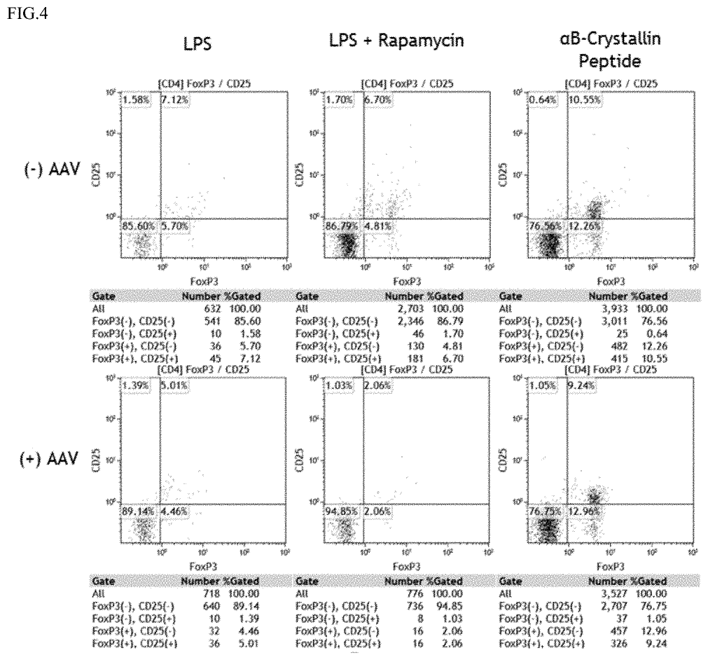

As shown in FIG. 3A, αA-crystallin (CRYAA) and αB-crystallin (CRYAB) were each observed to provide anti-inflammatory effects on human DCs.

Peripheral blood mononuclear cells (PBMCs) were isolated from healthy donor blood by density centrifugation over Lymphoprep. The resulting PBMCs were enriched for monocytes by adhesion to plastic, and subsequently induced to differentiate to DCs by culturing in RPMI-1640+10% fetal bovine serum (FBS) supplemented with granulocyte-macrophage colony-stimulating factor (GM-CSF; 800 U/ml) and interleukin four (IL-4; 500 U/ml) for 7 days at 37° C., 5% CO₂. Media was changed (75%) to fresh RPMI-1640+10% FBS and cytokines on days 3, 5 and 7. On day 7, cells were also treated with 10 μg/ml CRYAA, CRYAB, or phosphate-buffered saline (PBS) as a control and incubated for a further 24 hours at 37° C., 5% CO₂. The cells were then treated with 100 ng/ml MegaCD40L (Enzo Life Sciences) and incubated for a final 24 hours 37° C., 5% CO₂ before collecting the conditioned media. Relative cytokine concentrations in conditioned media from monocyte-derived dendritic cells (DCs) stimulated with CRYAA, CRYAB, or PBS was measured using a Proteome Profiler Human Cytokine Array Kit (R&D Systems) as per the manufacturer's instructions. Presented above are the changes in the expression of select cytokines (measured by densitometry), normalized to the PBS treated cells.

In embodiments, the effectiveness in providing immunotolerance is measured by detecting and quantifying IgM antibody levels, IgG antibody levels, or other antibody classes and serum IL-10 levels, TGF-β levels, IL-12 levels, TNF-α levels, and INF-γ levels, but not limiting to other inflammatory cytokine levels. In other embodiments, in vitro differentiation assays, such as quantifying Treg populations by flow cytometry were used to measure the effectiveness in providing immunotolerance. Other suitable assays, such as anti-antigen neutralizing antibody analysis, transgene expression level analysis, analysis of cytokine release, analysis of CD8/CD4 T-cell responses, adoptive transfer experiments, antigen specificity experiments, immunohistochemistry, T and B cell ELISpot assays, and quantitative reverse-transcription PCR (qRT-PCR) can be utilized.

As shown in FIG. 3B, αA-crystallin (CRYAA) and αB-crystallin (CRYAB) were each observed to provide anti-inflammatory effects on human M1 macrophages.

PBMCs were isolated from healthy donor blood by density centrifugation over Lymphoprep. The resulting PBMCs were enriched for monocytes by adhesion to plastic and induced to differentiate to M1 macrophages by culturing in RPMI-1640+10% fetal bovine serum (FBS) supplemented with GM-CSF (50 ng/ml) for 7 days at 37° C., 5% CO2. Media was changed (75%) to fresh RPMI-1640+10% FBS and cytokines on days 3, 5 and 7. On day 7, cells were also treated with 10 μg/ml αA-crystallin (CRYAA), αB-crystallin (CRYAB), or PBS (control) and incubated for 24 hours at 37° C., 5% CO₂. After 24 hours, conditioned media was collected from the treated cells. Relative cytokine concentrations in conditioned media from monocyte-derived dendritic cells (DCs) stimulated with CRYAA, CRYAB or PBS was measured using a Proteome Profiler Human Cytokine Array Kit (R&D Systems) as per the manufacturer's instructions. Presented are the changes in expression of select cytokines (measured by densitometry), normalized to the PBS-treated cells.

As shown in FIG. 4, regulatory T-cells were induced in αB-crystallin (CRYAB) peptide-treated dendritic cell co-culture.

PBMCs were isolated from healthy donor blood by density centrifugation over Lymphoprep. CD14+ monocytes were purified from the isolated PBMCs using an EasySep Human Monocyte Isolation Kit (Stemcell Technologies) and an EasySep Magnet, according to the manufacturer's instructions. From separately isolated PBMCs from the same blood donor, naive CD4⁺ T-cells were isolated using an EasySep Human Naive CD4⁺ T-Cell Isolation Kit (Stemcell Technologies) and EasySep magnet, according to the manufacturer's instructions. T-cells were stored in liquid nitrogen in Cryostor CS10 storage medium (Stemcell Technologies). The isolated CD14⁺ monocytes were immediately induced to differentiate into DCs by culturing in RPMI-1640+10% FBS supplemented with GM-CSF (800 U/ml) and IL-4 (500 U/ml) for 7 days at 37° C., 5% CO₂. Media was changed (75%) to fresh RPMI-1640+10% FBS and cytokines on days 3, 5 and 7. On day 7, immature DCs (imDCs) were counted and seeded in a 96-well plate at 10,000 imDCs per well. The plated imDCs were matured with 100 ng/ml LPS, 100 ng/ml LPS+100 ng/ml rapamycin, or 2 μg/ml αA-crystallin peptide (amino acids 73-92), in the presence or absence of 1×10⁹ AAV1 VG/ml. After 36 hours of maturation, DCs were washed twice with warm RPMI-1640+10% FBS, before re-suspending in the same medium. Previously isolated CD4⁺ naive T-cells were thawed and checked for viability with trypan blue. T-cells were diluted in RPMI-1640+10% FBS to a density of 250,000 cells/ml, and 100 μl of the suspension (25,000 T-cells) was added to each well of the 96-well plate containing the mature DCs. DCs and T-cells were co-cultured together at 37° C., 5% CO₂ for 5 days. At the end of the incubation, cells were stained with the following fluorophore-conjugated antibodies: anti-CD3-PE (clone UCHT1; Invitrogen), anti-CD4-PE-Cy7 (clone RPA-T4, eBioscience), anti-CD25-PerCP-Cy5.5 (clone BC96; eBioscience), and anti-FoxP3-FITC (clone PCH101; Invitrogen). FoxP3 staining was performed using a FoxP3/Transcription Factor Staining Buffer Set (eBioscience) according to the manufacturer's instructions. An Fc receptor-binding polyclonal antibody (eBioscience) was used to prevent non-specific binding of antibodies. The stained cells were re-suspended in PBS+0.5% bovine serum albumin (BSA)+2 mM ethylenediaminetetraacetic acid (EDTA), and flow cytometry was performed on a Guava EasyCyte Plus flow cytometer. Compensation was performed using UltraComp eBeads (Invitrogen) singly stained with each primary antibody. Analysis of the results was performed with Kaluza Analysis 2.1 (Beckman Coulter). FIG. 4 shows the expression of CD25 and FoxP3 gated on CD4⁺ cells. The frequency of each population within the CD4⁺ cells is overlaid in each quadrant.

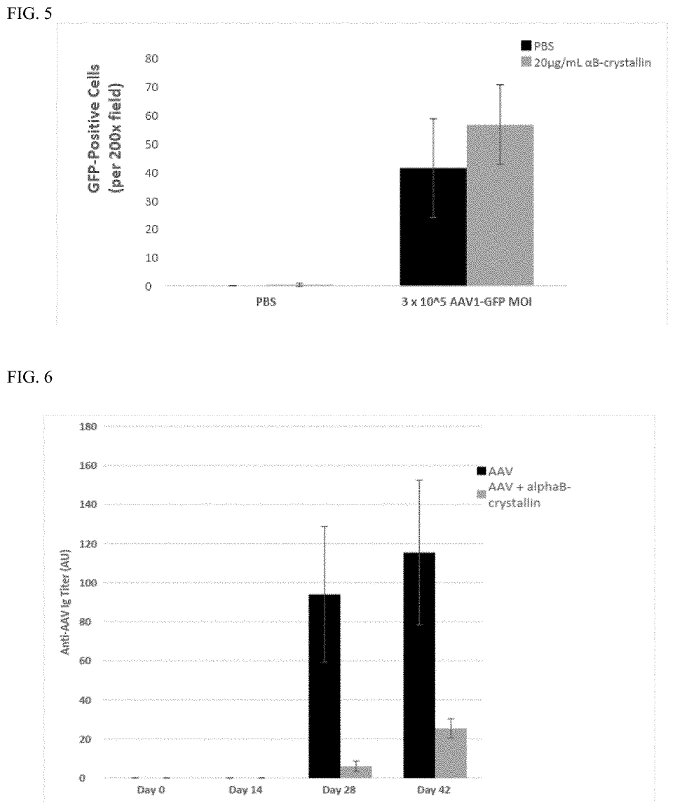

As shown in FIG. 5, αB-crystallin (CRYAB) did not interfere with AAV gene delivery.

HEK 293 cells were seeded at 10,000 cells per well in a 24-well plate, in 500 μl DMEM+10% FBS in the presence or absence of 20 μg/ml αB-crystallin (CRYAB) and AAV1-GFP (at an MOI of 3×10⁵). PBS was used as a control for both peptide treatment and viral transduction. Cells were incubated at 37° C., 5% CO₂ for 7 days. GFP-expressing cells were measured by acquiring images of three randomly selected 200× fields per well and counting the fluorescent cells.

HEK 293 cells were seeded at a density of 2,500 cells/well into 96-well plates, in 100 μl DMEM 10% FBS. Plates were incubated at 37° C., 5% CO₂ overnight. Day 42 sera from mice immunized 3 times (on days 0, 14, and 28) with PBS, αB-crystallin (CRYAB), AAV1, or AAV1+CRYAB was pooled (5 mice per group) and heat-inactivated at 56° C. for 30 min. The pooled serum was serially diluted into serum-free DMEM, and then mixed 1:1 with AAV1-GFP (2.5×10¹⁰ VG/ml in serum-free DMEM). The serum/AAV mixtures were incubated for 1 hr at 37° C. to allow for antibody binding. HEK 293 cells were treated with 25 μl of the appropriate serum/AAV mixture (multiplicity of infection: 1×10⁵) and incubated for a further 96 hours at 37° C., 5% CO₂. At the end of the incubation period, media was washed from the cells with cold PBS pH 7.4, and cells were trypsinized with trypsin-EDTA. The suspended cells were washed with PBS pH 7.4, pelleted by centrifugation at 500×g for 5 min, and resuspended in 200 μl PBS pH 7.4+0.5% BSA+2 mM EDTA. GFP Expression was detected by flow cytometry on a Guava EasyCyte Plus flow cytometer, with 10,000 events measured per well.

As shown in FIG. 6, mice injected with AAV+αB-crystallin (CRYAB) had reduced anti-AAV antibody titers compared to mice injected with AAV alone.

C57BL/6 mice aged 8-10 weeks old were injected with 50 μl of phosphate buffered saline solution (PBS) containing 1×10⁹ VG AAV1 in the presence or absence of 10 μg αB-crystallin (CRYAB) intramuscularly (IM) into the hind leg. Mice were injected on day 0, 14, and 28. Blood samples were taken on day 0, 28, and 42. Serum Anti-AAV1 Ig antibodies were detected in mouse sera by ELISA using 1×10¹⁰ VG/ml AAV1-null for plate coating and blocked with 3% bovine serum albumin (BSA) in PBS. Plates were revealed with donkey anti-mouse IgG (H+L) peroxidase conjugated and ABTS peroxidase substrate.

No detectable amounts of antibodies were observed in mice injected with PBS or αB-crystallin (CRYAB) alone.

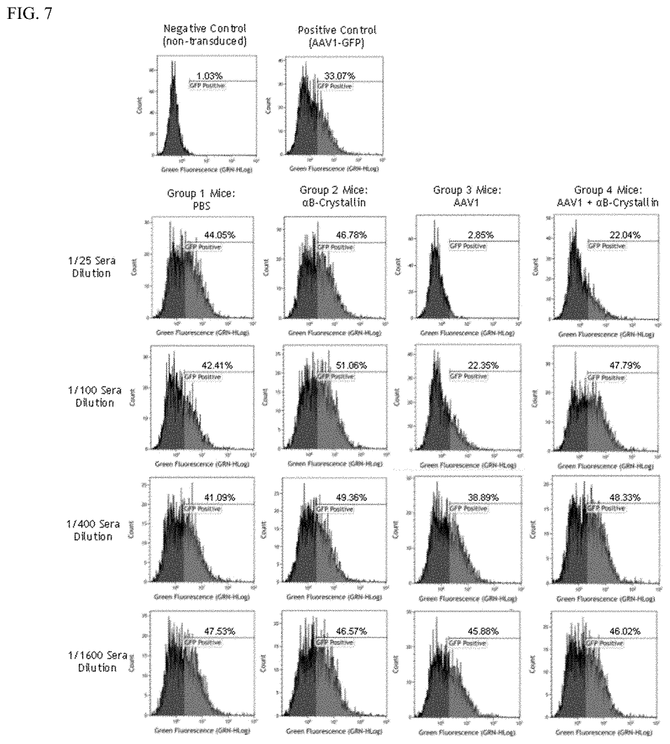

As shown in FIG. 7, αB-crystallin (CRYAB) reduced neutralizing antibodies.

HEK 293 cells were transduced (in triplicate) with AAV1-GFP and a range of dilutions of pooled serum from groups of mice previously immunized with PBS, αB-crystallin (CRYAB), AAV1, or a combination of CRYAB and AAV1. Cells were incubated for 4 days at 37° C., 5% CO₂ before measuring GFP expression by flow cytometry (10,000 events per well). Identified is the % GFP positive cells for each group.

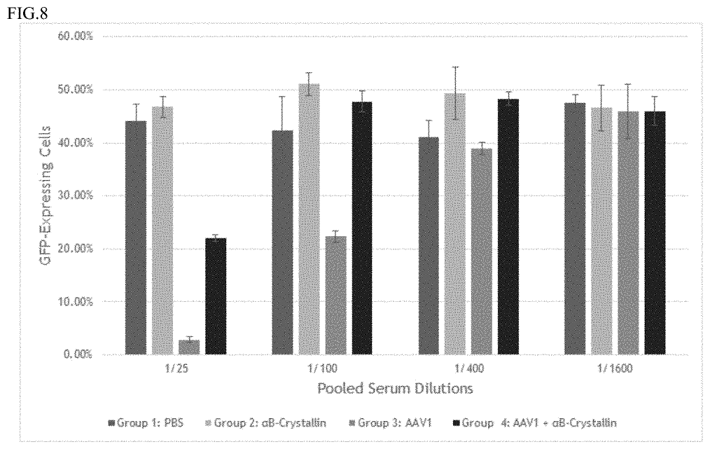

The average number of GFP expressing HEK293 cells in each treatment group after transduction with AAV1-GFP, as measured by flow cytometry, is presented in FIG. 8.

### Example 2: In the Presence of AAV1, αB-Crystallin (CRYAB) Induces Development of Regulatory T-Cells in Dendritic Cell Co-Cultures

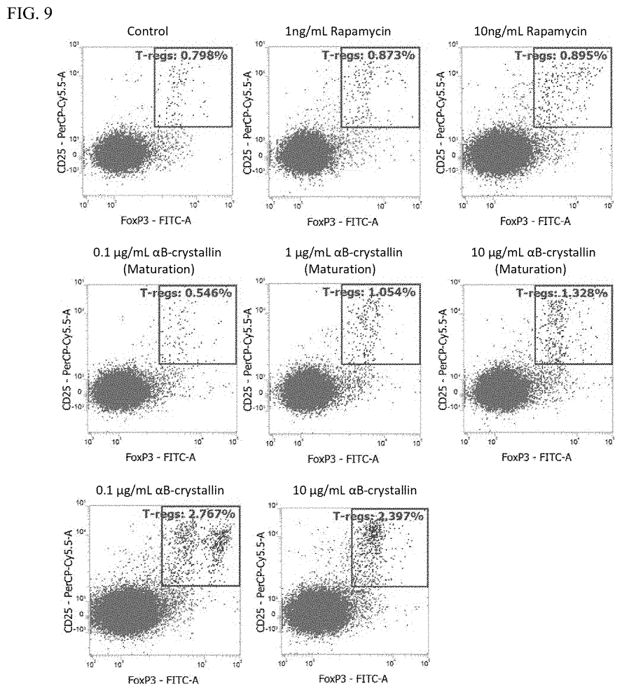

As shown in FIG. 9, αB-Crystallin (CRYAB) increased the frequency of CD25+/FoxP3+ Regulatory T cells (Tregs) within the CD3⁺/CD4⁺ (T-cell) population.

PBMCs were isolated from healthy donor blood by density centrifugation over Lymphoprep. CD14⁺ monocytes were purified from the isolated PBMCs using an EasySep Human Monocyte Isolation Kit (Stemcell Technologies) and an EasySep Magnet, according to the manufacturer's instructions. From separately isolated PBMCs from the same blood donor, naive CD4⁺ T-cells were isolated using an EasySep Human Naive CD4⁺ T-Cell Isolation Kit (Stemcell Technologies) and EasySep magnet, according to the manufacturer's instructions. T cells were stored in liquid nitrogen in Cryostor CS10 storage medium (Stemcell Technologies). The isolated CD14⁺ monocytes were immediately induced to differentiate into dendritic cells (DCs) by culturing in RPMI-1640+2% AB human serum supplemented with GM-CSF (800 U/ml) and IL-4 (500 U/ml) for 7 days at 37° C., 5% CO₂. Media was changed (75%) to fresh RPMI-1640+2% AB human serum and cytokines on days 3 and 5. During differentiation, some groups were treated with rapamycin (1 ng/ml or 10 ng/ml) or αB-crystallin (0.1 μg/ml or 10μg/ml) on days 3 and 5. On day 7, immature DCs (imDCs) were matured by changing to fresh RPMI-1640 supplemented with 2% AB human serum, 800 U/ml GM-CSF, 500 U/ml IL-4, maturation cocktail (long/ml IL-113, 10 ng/ml TNFα, and 1 μg/ml prostaglandin E2), and 1×10⁷ VG/ml AAV1-CAG-null as an antigen. DCs that had received rapamycin or αB-crystallin during differentiation were also treated with these agents during maturation, however several groups of DCs were also treated with αB-crystallin only during the maturation step.

After 48 hours, mature DCs were collected and seeded at 10,000 cells per well in a 96-well round bottom plate in fresh RPMI-1640 supplemented with 2% human serum. Previously-frozen naive CD4⁺T-cells were added to each well at a ratio of 10:1 T-cells to DCs (100,000 T-cells per well). Cells were co-cultured at 37° C., 5% CO₂ for 4 days. At the end of the co-culture period, cells were stained with the following fluorophore-conjugated antibodies: anti-CD3-PE (clone UCHT1; Invitrogen), anti-CD4-PE-Cy7 (clone RPA-T4, eBioscience), anti-CD25-PerCP-Cy5.5 (clone BC96; eBioscience), and anti-FoxP3-FITC (clone PCH101; Invitrogen). FoxP3 staining was performed using a FoxP3/Transcription Factor Staining Buffer Set (eBioscience) according to the manufacturer's instructions. An Fc receptor-binding polyclonal antibody (eBioscience) was used to prevent non-specific binding of antibodies. The stained cells were re-suspended in PBS+2% FBS+1 mM EDTA, and flow cytometry was performed on an Attune NxT flow cytometer (Thermo Fisher Scientific). Compensation was performed using UltraComp eBeads (Invitrogen) singly stained with each primary antibody. Presented are the frequency of CD25⁺/FoxP3⁺ (Treg) cells among the CD3⁺/CD4⁺ (T-cell) population. The frequency of each population among the CD4⁺ cells is overlaid in the “T-regs” gate.

### Example 3: Alpha ß-Crystallin (CRYAB) has a Tolerizing Effect in Mice as Demonstrated in a Reduction in Anti-AAV8 Antibody Titers

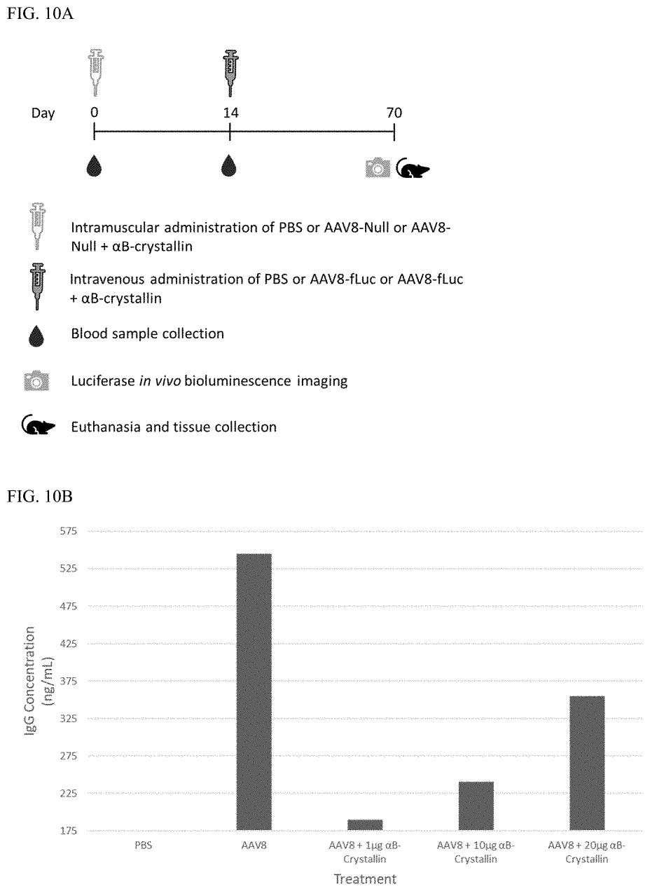

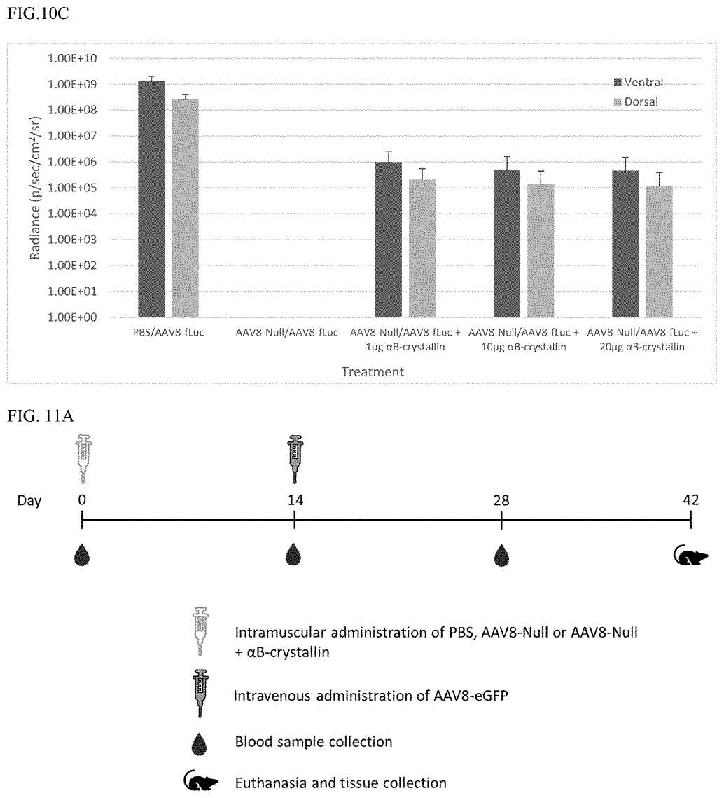

FIG. 10A is a schematic outlining a study that assessed the tolerizing effects of αB-crystallin (CRYAB) towards AAV in mice. This diagram indicates the timeline for injections, sampling, and luciferase activity measurements over the course of the 70-day study.

In these experiments, mice were immunized against AAV8 by intramuscular (IM) injection into the left hind leg of 2×10⁹ VG per mouse AAV8-null in 50 μl PBS on day 0 of the study to generate an anti-AAV8 immune response. Formulations for some groups of mice included αB-crystallin (CRYAB) at doses of 1 μg, 10 μg, or 20 μg per mouse to attenuate the anti-AAV8 response. Some mice were administered PBS alone on day 0 as a negative control. On day 14, mice were treated with 8×10¹⁰ VG per mouse of AAV8-fLuc intravenously, formulated in 50 μl PBS. Groups that received αB-crystallin in their day 0 immunization received the same dose of αB-crystallin on day 14, co-formulated with AAV8-fLuc. Some mice were administered PBS alone on day 14 as a negative control. Blood samples were collected from the mice on days 0 and 14, and subsequently processed to serum to assess the anti-AAV8 IgG response. Luciferase activity was measured by in vivo bioluminescence imaging. On day 70, mice were sacrificed and exsanguinated. Liver homogenates and splenocytes were collected from each mouse for further analysis.

As shown in FIG. 10B, in mice immunized with AAV8-Null and treated with AAV8-fLuc+αB-crystallin (CRYAB) had tolerizing effects in mice as demonstrated in a reduction in anti-AAV8 antibody titers at day 14.

FIG. 10C shows the preservation of luciferase expression in livers of mice that were challenged with AAV8-null and co-administered with αB-crystallin, and no detectable luciferase expression in livers of mice challenged with AAV8-null alone.

Mice were immunized intramuscularly with AAV8-Null in the presence or absence of αB-crystallin, and 14 days later were administered AAV8 bearing a luciferase transgene (AAV8-fLuc) intravenously, as described in FIG. 10A. Eight weeks following administration of AAV8-fLuc, mice were anesthetized with isoflurane and administered 150 mg/kg luciferin intraperitoneally (IP) 8 minutes before imaging. Luciferase activity was detected by measuring radiance (p/sec/cm²/sr) using an IVIS 13306 camera. Body temperature was maintained using a heated imaging chamber during this time.

Methods. Mice were injected with AAV8-Null and AAV8-fLuc in the presence or absence of αB-crystallin as described in FIG. 10A. Eight weeks after the administration of AAV8-fLuc, mice were anesthetized with isoflurane. Collected blood samples were rested at room temperature for 30 min before centrifuging for 8 min at 3,200×g. Serum was collected from the separated blood samples, aliquoted, and stored at −80° C. ELISA plates (96-well) were coated with 100 μl of 1×10¹¹ VG/ml AAV8-null overnight at 4° C. in a humidified chamber. Some wells were instead coated with goat anti-mouse IgG to prepare for standard curve generation. Plates were then washed three times with 200 μl wash buffer, and subsequently blocked with a 1% BSA blocking solution for 30 min at 37° C. Plates were again washed three times with 200 μl wash buffer before adding samples and standards (mouse serum with known IgG titers) in blocking buffer. Samples were added to the plate at an initial dilution of 1/20, and serially diluted 1/2 in the plate. Plates were incubated for two hours at 37° C. to allow for IgG binding. At the end of this period, plates were washed five times with wash buffer, and 100 μl of detection antibody (diluted 1/100,000) was added to each well. Plates were incubated for one hour at room temperature in a humidified chamber with the detection antibody. At the end of this period, plates were again washed five times with wash buffer. After washing, 100 μl of reconstituted TMB substrate was added to each well. Plates were then incubated for 30 min at room temperature, protected from light. The reaction was then stopped by adding 100 μl of stop solution to each well, and absorbance was measured at 450 nm on a SpectraMax iD3 Spectrophotometer (Molecular Devices). The lower limit of detection for the assay (LLOD) was 573 ng/ml. No antibodies were detected in any group on day zero.

### Example 4: Effect of Low Dose αB-Crystallin on Tolerization Effects

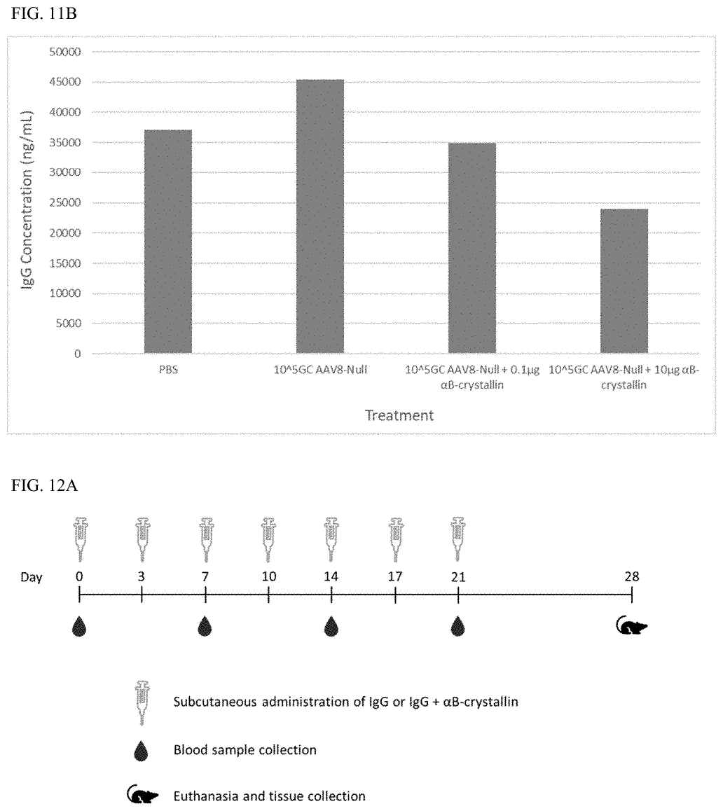

FIG. 11A is a schematic outlining a study designed to optimize the tolerizing effects of αB-crystallin (CRYAB) towards AAV in mice with various ratios of AAV to αB-crystallin. This diagram indicates the timeline for injections and blood sampling over the course of the 42-day study.

Mice were immunized against AAV8 by IM administration of 1×10⁵ VG AAV8-null (Vector Biolabs) formulated in 50 μl PBS on day 0 of the study. Formulations for some groups of mice included αB-crystallin at doses of 0.1 μg or 10 μg per mouse to attenuate the anti-AAV8 response. PBS was substituted for αB-crystallin in some groups as a negative control. On day 14, mice were treated with IV administrations of 8×10¹⁰ VG per mouse of AAV8-eGFP (Vector Biolabs) formulated in 50 μl PBS. Blood samples were collected from the mice on days 0, 14, and 28, and subsequently processed to serum to assess the anti-AAV8 IgG response that occurred. On day 42, mice were sacrificed and exsanguinated. Liver homogenates and splenocytes were collected from each mouse for further analysis.

Anti-AAV8 IgG in the serum of the mice described in FIG. 11A was measured by ELISA. Serum samples were collected from the mice on days 0, 14, and 28. Average antibody titers are plotted in FIG. 11B for each group. The lower limit of detection for the assay (LLOD) was 573 ng/ml. No antibodies were detected in any group on day 0 and day 14.

Methods. Mice were immunized with AAV8-null in the presence or absence of αB-crystallin at various ratios, as described in FIG. 11A. Fourteen days after immunization, mice were injected intravenously with AAV8-eGFP at a dose of 8×10⁸ VG per mouse. On day 42, mice were anesthetized with isoflurane and whole blood was collected via terminal heart puncture exsanguination using a 25 G ⅝″ needle. Blood was rested at room temperature for 30 min before centrifugation for 8 min at 3,200×g. Serum was collected from the separated blood samples, aliquoted, and stored at −80° C. ELISA plates (96-well) were coated with 100 μl of 1×10¹¹ VG/ml AAV8-null overnight at 4° C. in a humidified chamber. Some wells were instead coated with goat anti-mouse IgG to prepare for standard curve generation. Plates were then washed three times with 200 μl wash buffer, and subsequently blocked with a 1% BSA blocking solution for 30 min at 37° C. Plates were again washed three times with 200 μl wash buffer before adding samples and standards (mouse serum with known IgG titers) in blocking buffer. Samples were added to the plate at an initial dilution of 1/20, and serially diluted 1/2 in the plate. Plates were incubated for two hours at 37° C. to allow for IgG binding. At the end of this period, plates were washed five times with wash buffer, and 100 μl of detection antibody (diluted 1/100,000) was added to each well. Plates were incubated for one hour at room temperature in a humidified chamber with the detection antibody. At the end of this period, plates were again washed five times with wash buffer. After washing, 100 μl of reconstituted TMB substrate was added to each well. Plates were then incubated for 30 min at room temperature, protected from light. The reaction was then stopped by adding 100 μl of stop solution to each well, and absorbance was measured at 450 nm on a SpectraMax iD3 Spectrophotometer (Molecular Devices).

### Example 5: Tolerization of Mice Towards Human Immunoglobulin G in the Presence of αB-Crystallin

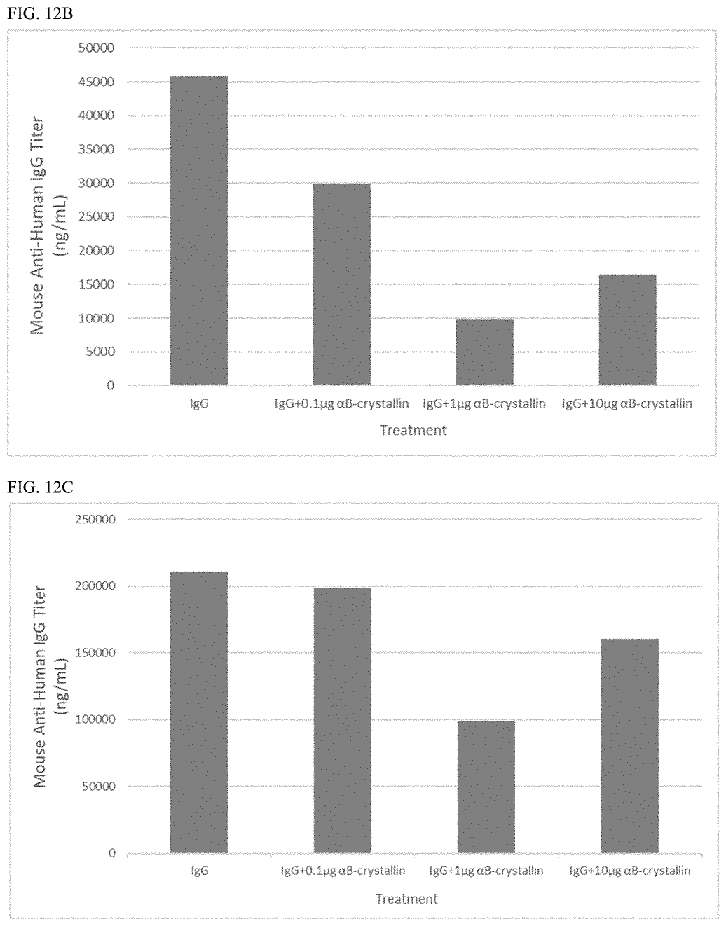

FIG. 12A is a schematic outlining a study designed to assess the tolerizing effects of αB-crystallin (CRYAB) towards human IgG in mice. Mice were immunized by subcutaneous (SC) administration of human IgG at several timepoints, in the presence or absence of a range of αB-crystallin concentrations. This diagram indicates the timeline for injections and blood sampling over the course of the 28-day study.

Mice were immunized against human IgG by SC administration of 10 μg polyclonal human IgG (Sigma) formulated in 50 μl PBS on days 0, 3, 7, 10, 14, 17, and 21 of the study to generate an anti-human IgG immune response. Formulations for some groups of mice included αB-crystallin (CRYAB) at doses of 0.1 μg, 1 μg, or 10 μg per mouse to attenuate the anti-human IgG response. PBS was substituted for αB-crystallin in some groups as a negative control. Blood samples were collected from the mice on days 0, 7, 14, and 21, and subsequently processed to serum to assess the anti-human IgG response that occurred. On day 28, mice were sacrificed and exsanguinated. Splenocytes were collected from each mouse for further analysis.

FIG. 12B and FIG. 12C show mouse anti-human IgG in the serum of the mice described (at day 14 and day 21, respectively) as measured by ELISA. Serum samples were collected from the mice on days 0, 7, 14 and 21. Average antibody titers plus standard deviation are plotted for each group. The lower limit of detection for the assay (LLOD) was 133 ng/ml. No antibodies were detected in any group on day 0 and day 7.

Methods. Blood samples were collected at several intervals via facial veins using 21 G 1″ disposable needles into 200 μL Z-Gel Microtubes (Sarstedt). Blood was rested at room temperature for 30 min before centrifugation for 8 min at 3,200×g. Serum was collected from the separated blood samples, aliquoted, and stored at −80° C. ELISA plates (96-well) were coated with 4 μg/mL human IgG overnight at 4° C. in a humidified chamber. Some wells were instead coated with goat anti-mouse IgG to prepare for standard curve generation. Plates were then washed three times with 2004 wash buffer, and subsequently blocked with a 1% BSA blocking solution for 30 min at 37° C. Plates were again washed three times with 2000_, wash buffer before adding samples and reference standards (mouse serum with known IgG titers) in blocking buffer. Samples were added to the plate at an initial dilution of 1/20, and serially diluted 1/2 in the plate. Plates were incubated for two hours at 37° C. to allow for IgG binding. At the end of this period, plates were washed five times with wash buffer, and 1004 of detection antibody (diluted 1/100,000) was added to each well. Plates were incubated for one hour at room temperature in a humidified chamber with the detection antibody. At the end of this period, plates were again washed five times with wash buffer. After washing, 1004 of reconstituted TMB substrate was added to each well. Plates were then incubated for 30 min at room temperature, protected from light. The reaction was then stopped by adding 1004 of stop solution to each well, and absorbance was measured at 450 nm on a SpectraMax iD3 Spectrophotometer (Molecular Devices).

### Example 6: Methods for Priming an Effective Response

In this example, a subject is initially administered a composition comprising a therapeutic agent at a low dosage.

Here, the subject is administered a composition comprising a therapeutic agent at a dose known or believed to be ineffective in producing a therapeutic response. The therapeutic agent is a nucleic acid encapsulated in a viral vector. Alternately, the composition comprises the therapeutic agent as a nucleic acid (without a packaging component) and pharmaceutically-acceptable excipients.

The subject may be administered additional subsequent compositions also at doses of the therapeutic agent known or believed to be ineffective in producing a therapeutic response.

Before administering the first (or subsequent) composition comprising a sub-effective dose of the therapeutic agent, the subject is administered a composition comprising a heat shock protein. Alternately, concurrently with administering the first (or subsequent) composition comprising a sub-effective dose of the therapeutic agent, the subject is administered a composition comprising a heat shock protein.

Administration is by intravenous injection or infusion.

A first blood sample is collected from the subject before administering any compositions, a second blood sample is collected from the subject after administering the first composition, and other blood samples are collected from the subject after each subsequent administration.

Each blood sample is assayed for evidence of a therapeutic benefit. If a blood sample collected after administering a composition comprising a sub-effective dose of the therapeutic agent demonstrates evidence of a therapeutic benefit, then the subject may continue to receive the same dose.

Without wishing to be bound by theory, such a result would suggest that the heat shock protein provides a favorable in vivo environment that allows efficacy of the therapeutic agent at lower doses, such as those with less efficacy and those in a sub-effective range (when not administered with a heat shock protein). By administering lower doses, the frequency of the dosing of the therapeutic agent may be reduced and/or the safety profile improved.

If necessary or desirable, the subject may be further administering one or more doses of the therapeutic agent at a higher dose, e.g., at a dose effective in producing a therapeutic response in the absence of a heat shock protein.

### Example 7: Methods Providing Peptide/Protein-Based Therapeutic Agents

In this example, a subject is administered a composition comprising a heat shock protein and comprising a peptide/protein-based therapeutic agent or a pair of compositions with a first composition comprising a heat shock protein and a second composition comprising a peptide/protein-based therapeutic agent.

Here, the subject is administered a therapeutic agent that is peptide-based or protein-based. Examples of such therapeutic agents include antibodies, peptides/proteins comprising antigen binding fragments, antibody-based drugs (e.g., antibody-drug conjugates (ADC)), Fc fusion proteins, anticoagulants, blood factors, bone morphogenetic proteins, engineered protein scaffolds, enzymes, growth factors, hormones, interferons, interleukins, and thrombolytics. Also, included are gene-editing proteins, e.g., a CRISPR-associated protein 9 (Cas9), a Transcription Activator-Like Effector Nucleases (TALEN), or a Zinc Finger Nuclease (ZFN).

When the subject is administered a pair of compositions (with a first composition comprising a heat shock protein and a second composition comprising a peptide/protein-based therapeutic agent), the subject is either administered the first composition before the second composition, administered the first composition after the second composition comprising the therapeutic agent, or administered the first composition concurrent with the second composition comprising the therapeutic agent.

Alternately, a subject is administered a third composition comprising a heat shock protein and comprising a peptide/protein-based therapeutic agent before, after, or concurrent with a first composition or a second composition, as described herein.

Administration is by intravenous injection or infusion, with a dose depending on the quantity of each composition needing to be administered.

A first blood sample is collected from the subject before administering any compositions, a second blood sample is collected from the subject after administering an earlier composition, and a third blood sample is collected from the subject after administering a later composition. The blood samples are screened for the presence and amounts of various markers that indicate inflammation. Comparisons are made among results from the collected blood samples. In certain cases, only a blood sample is collected from the subject after administering the composition(s) and comparisons are made between results from the collected sample(s) and historical controls.

If a blood sample collected after administering a composition comprising the heat shock protein indicates an increase in the presence and amounts of various markers that indicate inflammation, either a subsequent composition will comprise a higher dosage of the heat shock protein and/or a subsequent composition will comprise a lower dosage of the therapeutic agent. Alternately, if a blood sample collected after administering the composition comprising a heat shock protein indicates a decrease in the amounts of various markers that indicate inflammation, either a subsequent composition will comprise a lower dosage of the heat shock protein and/or a subsequent composition will comprise a higher dosage of the therapeutic agent.

Additionally, each blood sample may be assayed for evidence of a therapeutic benefit.

### Example 8: Methods Providing Co-Therapies with Immune Suppressants

In this example, a subject is administered a co-therapy comprising an immune suppressant which enhances an immune tolerance effect.

Here, a subject is administered an immune suppressant, a heat shock protein, and a therapeutic agent. All three of these ingredients may be in the same composition, two of the ingredients may be in one composition with the third ingredient in a second composition, or each ingredient may be in its own composition.

Examples of immunosuppressants include cyclosporine, prednisone, dexamethasone, hydrocortisone, methotrexate, azathioprine, mercaptopurine, dactinomycin, mycophenolate, mitomycin C, bleomycin, mithramycin, rapamycin, tacrolimus, deforolimus, everolimus, temsirolimus, zotarolimus, biolimus A9, pemecrolimus, voclosporin and sirolimus. Also, antibody therapeutics that modulate immune system are used. Non-limiting examples include use of an anti-TNFalpha antibody (e.g., adalimumab), an anti-CD20 antibody (e.g., rituximab), and anti-human thymocyte immunoglobulins (e.g., Thymoglobulin).

The immunosuppressant is administered in any combination with the heat shock protein and with the therapeutic agent. Thus, the immunosuppressant is administered before, after, or concurrent with the heat shock protein and the immunosuppressant is administered before, after, or concurrent with the therapeutic agent.

Administration route and dosage is as appropriate for the ingredient (immunosuppressant, heat shock protein, and therapeutic agent). For example, some immunosuppressants may be administered orally and other immunosuppressants may be administered by intravenous injection or infusion.

Blood samples are collected from the subject before administering a composition and/or after administering a composition. The blood samples are screened for the presence and amounts of various markers that indicate inflammation. Comparisons are made among results from the collected blood samples. In certain cases, only a blood sample is collected from the subject after administering the composition(s) and comparisons are made between results from the collected sample(s) and historical controls.

If a blood sample collected after administering the immunosuppressant indicates an increase in the presence and amounts of various markers that indicate inflammation, either a subsequent administration will comprise a higher dosage of the immunosuppressant, a subsequent administration will comprise a higher dosage of the shock protein, and/or a subsequent administration will comprise a lower dosage of the therapeutic agent. Alternately, if a blood sample collected after administering the immunosuppressant indicates a decrease in the amounts of various markers that indicate inflammation, either a subsequent administration will comprise a lower dosage of the immunosuppressant, a subsequent administration will comprise a lower dosage of the shock protein, and/or a subsequent administration will comprise a higher dosage of the therapeutic agent.

Additionally, each blood sample may be assayed for evidence of a therapeutic benefit.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

1.-74. (canceled)

75. A method for generating an immune tolerizing effect against a therapeutic agent administered to a subject comprising:
administering to the subject a therapeutic agent; and
administering to the subject an effective amount of a heat shock protein to generate an immune tolerizing effect against the therapeutic agent in the subject.

76. The method of claim 75, wherein the heat shock protein is αB-crystallin (CRYAB), αA-crystallin (CRYAA), HSP60, HSP70, HSP72, HSP84, HSP90, HSP104, GP96, HSP33, HSP27, HSP22, HSP20, HSP12, HSP10, or HSP7, or a functional fragment thereof.

77. The method of claim 75, wherein the heat shock protein comprises a small heat shock protein (sHsp) or a functional fragment thereof.

78. The method of claim 77, wherein the sHsp comprises one or more features selected from (i) a subunit molecular mass between about 12 and about 43 kDa, (ii) an α-crystallin domain, (iii) an N-terminal domain and (iv) C-terminal extension.

79. The method of claim 77, wherein the heat shock protein is αB-crystallin (CRYAB).

80. The method of claim 79, wherein said CRYAB comprises a sequence selected from the group consisting of SEQ ID NO: 18 to 25.

81. The method of claim 79, wherein said CRYAB comprises a sequence of SEQ ID NO: 18 or a sequence that is at least 65%, 70%, 75%, 80%, 85%, 90%, 95%, 96%, 97%, 98%, or 99% identical to SEQ ID NO: 18.

82. The method of claim 75, wherein the therapeutic agent comprises a nucleic acid, a peptide, a protein, a compound, a chemotherapeutic, or a cell, or any combination thereof.

83. The method of claim 82, wherein the nucleic acid is DNA or RNA.

84. The method of claim 75, wherein the therapeutic agent comprises a packaging component.

85. The method of claim 84, wherein the packaging component is a particle, a viral vector, a virus, or a virus-like particle.

86. The method of claim 85, wherein the viral vector is a lentivirus or an adeno-associated virus (AAV).

87. The method of claim 86, wherein the AAV is selected from the group consisting of AAV1, AAV2, AAV4, AAV5, AAV6, AAV7, AAV8, AAV9, AAV2/5, AAV2/2, AAV-DJ, and AAV-DJ8, or any combination thereof.

88. The method of claim 75, wherein the immune tolerizing effect comprises the modulation of the expression of one or more of IL-10, TGFβ, IL-35, perforin, granzyme, IDO1, IFNγ, PTX3, TNFAIP6, SLAMF1, CCL20, ANGPTL4, CCL19, BIRC3, ADM, TSG6, PD-L1, PD-1, and LAG3 and/or the immune tolerizing effect comprises a change in the number and/or ratio regulatory T cells, Tr1 cells, and/or exhausted T cells.

89. The method of claim 75, wherein the subject is a mammal.

90. The method of claim 89, wherein the mammal is a human.

91. The method of claim 75, further comprising administering to the subject one or more subsequent doses of the therapeutic agent, wherein the one or more subsequent doses is effective to generate a therapeutic response.

92. The method of claim 91, wherein the therapeutic agent is a gene therapy.

93. The method of claim 91, wherein the administration of the heat shock protein reduces, prevents, or alleviates an immune response to the one or more subsequent doses of the therapeutic agent.

94. The method of claim 91, wherein the administration of the heat shock protein reduces, prevents, or alleviates inactivation of the one or more subsequent doses of the therapeutic agent.

95. A pharmaceutical composition comprising an immune tolerizing effective amount of a heat shock protein, wherein the immune tolerizing effective amount of the heat shock protein is capable of reducing or inhibiting an immune response to a therapeutic agent administered to a subject.

96. The pharmaceutical composition of claim 95, wherein the heat shock protein comprises a small heat shock protein (sHsp) or a functional fragment thereof.

97. The pharmaceutical composition of claim 95, wherein the heat shock protein is CRYAB.

98. The pharmaceutical composition of claim 95, further comprising a therapeutic agent selected from the group consisting of a nucleic acid, a peptide, a protein, a compound, a chemotherapeutic, and a cell, or any combination thereof.

99. The pharmaceutical composition of claim 98, wherein the cell is selected from the group consisting of a tumor-infiltrating lymphocyte (TIL) cell, an engineered T cell receptor (TCR) cell, a chimeric antigen receptor (CAR) T cell, a Treg cell, a CAR-Treg cell, dendritic cell, a natural killer (NK) cell, and a stem cell.

100. A kit comprising the pharmaceutical composition of claim 95 and a second pharmaceutical composition comprising a therapeutic agent selected from the group consisting of a nucleic acid, a peptide, a protein, a compound, a chemotherapeutic, and a cell, or any combination thereof.

101. The kit of claim 100, wherein the cell is selected from the group consisting of a tumor-infiltrating lymphocyte (TIL) cell, an engineered T cell receptor (TCR) cell, a chimeric antigen receptor (CAR) T cell, a Treg cell, a CAR-Treg cell, dendritic cell, a natural killer (NK) cell, and a stem cell.

102. The kit of claim 100, wherein the therapeutic agent comprises a nucleic acid selected from the group consisting of DNA, RNA and a nucleic acid packaged in a viral vector.


SEQUENCE LISTING (52 sequences)

SEQ ID NO: 1 - protein, 858 aa, Homo sapiens
MSVVGLDVGSQSCYIAVARAGGIETIANEFSDRCTPSVISFGSKNRTIGVAAKNQQITHANNTVSNFKRFHGRAFNDPFIQKEKENLSYDLVPLKNGGVGIKVMYMGEEHLFSVEQITAMLLTKLKETAENSLKKPVTDCVISVPSFFTDAERRSVLDAAQIVGLNCLRLMNDMTAVALNYGIYKQDLPSLDEKPRIVVFVDMGHSAFQVSACAFNKGKLKVLGTAFDPFLGGKNFDEKLVEHFCAEFKTKYKLDAKSKIRALLRLYQECEKLKKLMSSNSTDLPLNIECFMNDKDVSGKMNRSQFEELCAELLQKIEVPLYSLLEQTHLKVEDVSAVEIVGGATRIPAVKERIAKFFGKDISTTLNADEAVARGCALQCAILSPAFKVREFSVTDAVPFPISLIWNHDSEDTEGVHEVFSRNHAAPFSKVLTFLRRGPFELEAFYSDPQGVPYPEAKIGRFVVQNVSAQKDGEKSRVKVKVRVNTHGIFTISTASMVEKVPTEENEMSSEADMECLNQRPPENPDTDKNVQQDNSEAGTQPQVQTDAQQTSQSPPSPELTSEENKIPDADKANEKKVDQPPEAKKPKIKVVNVELPIEANLVWQLGKDLLNMYIETEGKMIMQDKLEKERNDAKNAVEEYVYEFRDKLCGPYEKFICEQDHQNFLRLLTETEDWLYEEGEDQAKQAYVDKLEELMKIGTPVKVRFQEAEERPKMFEELGQRLQHYAKIAADFRNKDEKYNHIDESEMKKVEKSVNEVMEWMNNVMNAQAKKSLDQDPVVRAQEIKTKIKELNNTCEPVVTQPKPKIESPKLERTPNGPNIDKKEEDLEDKNNFGAEPPHQNGECYPNEKNSVNMDLD

SEQ ID NO: 2 - protein, 858 aa, Mus sp.
MSVVGLDVGSQSCYIAVARAGGIETIANEFSDRCTPSVISFGSKNRTIGVAAKNQQITHANNTVSSFKRFHGRAFNDPFIQKEKENLSYDLVPMKNGGVGIKVMYMDEEHFFSVEQITAMLLTKLKETAENNLKKPVTDCVISVPSFFTDAERRSVLDAAQIVGLNCLRLMNDMTAVALNYGIYKQDLPNAEEKPRVVVFVDMGHSSFQVSACAFNKGKLKVLGTAFDPFLGGKNFDEKLVEHFCAEFKTKYKLDAKSKIRALLRLHQECEKLKKLMSSNSTDLPLNIECFMNDKDVSGKMNRSQFEELCAELLQKIEVPLHSLMAQTQLKAEDVSAIEIVGGATRIPAVKERIAKFFGKDVSTTLNADEAVARGCALQCAILSPAFKVREFSVTDAVPFPISLVWNHDSEETEGVHEVFSRNHAAPFSKVLTFLRRGPFELEAFYSDPQGVPYPEAKIGRFVVQNVSAQKDGEKSRVKVKVRVNTHGIFTISTASMVEKVPTEEEDGSSLEADMECPNQRPTESSDVDKNIQQDNSEAGTQPQVQTDGQQTSQSPPSPELTSEESKTPDADKANEKKVDQPPEAKKPKIKVVNVELPVEANLVWQLGRDLLNMYIETEGKMIMQDKLEKERNDAKNAVEECVYEFRDKLCGPYEKFICEQEHEKFLRLLTETEDWLYEEGEDQAKQAYIDKLEELMKMGTPVKVRFQEAEERPKVLEELGQRLQHYAKIAADFRGKDEKYNHIDESEMKKVEKSVNEVMEWMNNVMNAQAKRSLDQDPVVRTHEIRAKVKELNNVCEPVVTQPKPKIESPKLERTPNGPNIDKKEDLEGKNNLGAEAPHQNGECHPNEKGSVNMDLD

SEQ ID NO: 3 - protein, 858 aa, Rattus sp.
MSVVGLDVGSQSCYIAVARAGGIETIANEFSDRCTPSVISFGPKNRTIGVAAKNQQITHANNTVSSFKRFHGRAFNDPFIQKEKENLSYDLVPMKNGGVGIKVMYMDEDHLFSVEQITAMLLTKLKETAENNLKKPVTDCVISVPSFFTDAERRSVLDAAQIVGLNCLRLMNDMTAVALNYGIYKQDLPNADEKPRVVVFVDMGHSSFQVSACAFNKGKLKVLGTAFDPFLGGKNFDEKLVEHFCAEFKTKYKLDAKSKIRALLRLHQECEKLKKLMSSNSTDLPLNIECFMNDKDVSAKMNRSQFEELCAELLQKIEVPLHLLMEQTHLKTEEVSAIEIVGGATRIPAVKERIARFFGKDVSTTLNADEAVARGCALQCAILSPAFKVREFSVTDAVPFPISLVWNHDSEETEGVHEVFSRNHAAPFSKVLTFLRRGPFELEAFYSDPQAVPYPEAKIGRFVVQNVSAQKDGEKSKVKVKVRVNTHGIFTISTASMVEKVPTEEEDGSSVEADMECPNQKPAESSDVDKNIQQDNSEAGTQPQVQTDGQQTSQSPPSPELTSEENKIPDADKANEKKVDQPPEAKKPKIKVVNVELPVEANLVWQLGRDLLNMYIETEGKMIMQDKLEKERNDAKNAVEECVYEFRDKLCGPYEKFICEQEHEKFLRLLTETEDWLYEEGEDQAKQAYIDKLEELMKMGTPVKVRFQEAEERPRVLEELGQRLQHYAKIAADFRGKDEKYNHIDESEMKKVEKSVNEVMEWMNNVMNAQAKRSLHQDPVVRTHEISAKVKELNNVCEPVVTQPKPKIESPKLERTPNGPNMDKKEDLEGKSNLGADAPHQNGECHPNEKGSVSMDLD

SEQ ID NO: 4 - protein, 724 aa, Homo sapiens
MPEEVHHGEEEVETFAFQAEIAQLMSLIINTFYSNKEIFLRELISNASDALDKIRYESLTDPSKLDSGKELKIDIIPNPQERTLTLVDTGIGMTKADLINNLGTIAKSGTKAFMEALQAGADISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVAAEEPNAAVPDEIPPLEGDEDASRMEEVD

SEQ ID NO: 5 - protein, 733 aa, Rattus sp.
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEEKEEEKEKEEKESDDKPEIEDVGSDEEEEEKKDGDKKKKKKIKEKYIDQEELNKTKPIWTRNPDDITNEEYGEFYKSLTNDWEEHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYFITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTVDDTSAAVTEEMPPLEGDDDTSRMEEVD

SEQ ID NO: 6 - protein, 724 aa, Rattus sp.
MPEEVHHGEEEVETFAFQAEIAQLMSLIINTFYSNKEIFLRELISNASDALDKIRYESLTDPSKLDSGKELKIDIIPNPQERTLTLVDTGIGMTKADLINNLGTIAKSGTKAFMEALQAGADISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKEDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVTAEEPSAAVPDEIPPLEGDEDASRMEEVD

SEQ ID NO: 7 - protein, 709 aa, Saccharomyces cerevisiae [sequence omitted]

SEQ ID NO: 8 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 9 - protein, 641 aa, Homo sapiens; identical to SEQ ID NO: 8

SEQ ID NO: 10 - protein, 641 aa, Mus sp.
MAKNTAIGIDLGTTYSCVGVFQHGKVEIIANDQGNRTTPSYVAFTDTERLIGDAAKNQVALNPQNTVFDAKRLIGRKFGDAVVQSDMKHWPFQVVNDGDKPKVQVNYKGESRSFFPEEISSMVLTKMKEIAEAYLGHPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVSHFVEEFKRKHKKDISQNKRAVRRLRTACERAKRTLSSSTQASLEIDSLFEGIDFYTSITRARFEELCSDLFRGTLEPVEKALRDAKMDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQTFTTYSDNQPGVLIQVYEGERAMTRDNNLLGRFELSGIPPAPRGVPQIEVTFDIDANGILNVTATDKSTGKANKITITNDKGRLSKEEIERMVQEAERYKAEDEVQRDRVAAKNALESYAFNMKSAVEDEGLKGKLSEADKKKVLDKCQEVISWLDSNTLADKEEFVHKREELERVCSPIISGLYQGAGAPGAGGFGAQAPKGASGSGPTIEEVD

SEQ ID NO: 11 - protein, 642 aa, Mus sp.
MAKNTAIGIDLGTTYSCVGVFQHGKVEIIANDQGNRTTPSYVAFTDTERLIGDAAKNQVALNPQNTVFDAKRLIGRKFGDAVVQSDMKHWPFQVVNDGDKPKVQVNYKGESRSFFPEEISSMVLTKMKEIAEAYLGHPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVSHFVEEFKRKHKKDISQNKRAVRRLRTACERAKRTLSSSTQASLEIDSLFEGIDFYTSITRARFEELCSDLFRGTLEPVEKALRDAKMDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQTFTTYSDNQPGVLIQVYEGERAMTRDNNLLGRFELSGIPPAPRGVPQIEVTFDIDANGILNVTATDKSTGKANKITITNDKGRLSKEEIERMVQEAERYKAEDEVQRDRVAAKNALESYAFNMKSAVEDEGLKGKLSEADKKKVLDKCQEVISWLDSNTLADKEEFVHKREELERVCSPIISGLYQGAGAPGAGGFGAQAPPKGASGSGPTIEEVD

SEQ ID NO: 12 - protein, 340 aa, Homo sapiens
MGKDYYQTLGLARGASDEEIKRAYRRQALRYHPDKNKEPGAEEKFKEIAEAYDVLSDPRKREIFDRYGEEGLKGSGPSGGSGGGANGTSFSYTFHGDPHAMFAEFFGGRNPFDTFFGQRNGEEGMDIDDPFSGFPMGMGGFTNVNFGRSRSAQEPARKKQDPPVTHDLRVSLEEIYSGCTKKMKISHKRLNPDGKSIRNEDKILTIEVKKGWKEGTKITFPKEGDQTSNNIPADIVFVLKDKPHNIFKRDGSDVIYPARISLREALCGCTVNVPTLDGRTIPVVFKDVIRPGMRRKVPGEGLPLPKTPEKRGDLIIEFEVIFPERIPQTSRTVLEQVLPI

SEQ ID NO: 13 - protein, 340 aa, Mus sp.
MGKDYYQTLGLARGASDDEIKRAYRRQALRYHPDKNKEPGAEEKFKEIAEAYDVLSDPRKREIFDRYGEEGLKGGSPSGGSSGGANGTSFSYTFHGDPHAMFAEFFGGRNPFDTFFGQRNGEEGMDIDDTFSSFPMGMGGFTNMNFGRSRPSQEPTRKKQDPPVTHDLRVSLEEIYSGCTKKMKISHKRLNPDGKSIRNEDKILTIEVKRGWKEGTKITFPKEGDQTSNNIPADIVFVLKDKPHNIFKRDGSDVIYPARISLREALCGCTVNVPTLDGRTIPVVFKDVIRPGMRRKVPGEGLPLPKTPEKRGDLVIEFEVIFPERIPVSSRTILEQVLPI

SEQ ID NO: 14 - protein, 357 aa, Rattus sp.
MVDYYEVLGVQRHASPEDIKKAYRKQALKWHPDKNPENKEEAERKFKQVAEAYEVLSDAKKRDIYDKYGKEGLNGGGGGGGSHFDSPFEFGFTFRNPDDVFREFFGGRDPFSFDFFEDPFDDFFGNRRGPRGSRSRGAGSFFSAFSGFPSFGSGFPAFDTGFTPFGSLGHGGLTSFSSASFGGSGMGNFKSISTSTKIVNGKKITTKRIVENGQERVEVEEDGQLKSLTINGVADENALAEECRRRGQPTPALAPGPAPAPARVPSQARPPTPAPTPAPAQTPAPSVSTRPQKPPRPAPTAKLVSKSNWEDEEQDRQRVPGNCDAPMTSAGLKEGGKRKKQKQKEDSKKKKSTKGNH

SEQ ID NO: 15 - protein, 205 aa, Homo sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 16 - protein, 206 aa, Rattus sp.
MTERRVPFSLLRSPSWEPFRDWYPAHSRLFDQAFGVPRFPDEWSQWFSSAGWPGYVRPLPAATAEGPAAVTLARPAFSRALNRQLSSGVSEIRQTADRWRVSLDVNHFAPEELTVKTKEGVVEITGKHEERQDEHGYISRCFTRKYTLPPGVDPTLVSSSLSPEGTLTVEAPLPKAVTQSAEITIPVTFEARAQIGGPESEQSGAK

SEQ ID NO: 17 - protein, 209 aa, Mus sp.
MTERRVPFSLLRSPSWEPFRDWYPAHSRLFDQAFGVPRLPDEWSQWFSAAGWPGYVRPLPAATAEGPAAVTLAAPAFSRALNRQLSSGVSEIRQTADRWRVSLDVNHFAPEELTVKTKEGVVEITGKHEERQDEHGYISRCFTRKYTLPPGVDPTLVSSSLSPEGTLTVEAPLPKAVTQSAEITIPVTFEARAQIGGPEAGKSEQSGAK

SEQ ID NO: 18 - protein, 175 aa, Homo sapiens
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK

SEQ ID NO: 19 - protein, 175 aa, Bos taurus
MDIAIHHPWIRRPFFPFHSPSRLFDQFFGEHLLESDLFPASTSLSPFYLRPPSFLRAPSWIDTGLSEMRLEKDRFSVNLDVKHFSPEELKVKVLGDVIEVHGKHEERQDEHGFISREFHRKYRIPADVDPLAITSSLSSDGVLTVNGPRKQASGPERTIPITREEKPAVTAAPKK

SEQ ID NO: 20 - protein, 175 aa, Rattus sp.
MDIAIHHPWIRRPFFPFHSPSRLFDQFFGEHLLESDLFSTATSLSPFYLRPPSFLRAPSWIDTGLSEMRMEKDRFSVNLDVKHFSPEELKVKVLGDVIEVHGKHEERQDEHGFISREFHRKYRIPADVDPLTITSSLSSDGVLTVNGPRKQASGPERTIPITREEKPAVTAAPKK

SEQ ID NO: 21 - protein, 175 aa, Mus sp.
MDIAIHHPWIRRPFFPFHSPSRLFDQFFGEHLLESDLFSTATSLSPFYLRPPSFLRAPSWIDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVAAAPKK

SEQ ID NO: 22 - protein, 174 aa, Gallus gallus
MDITIHNPLIRRPLFSWLTPSRIFDQIFGEHLQESELLPTSPSLSPFLMRSPFFRMPSWLETGLSEMRLEKDKFSVNLDVKHFSPEELKVKVLGDMIEIHGKHEERQDEHGFIAREFSRKYRIPADVDPLTITSSLSLDGVLTVSAPRKQSDVPERSIPITREEKPAIAGSQRK

SEQ ID NO: 23 - protein, 175 aa, Oryctolagus cuniculus
MDIAIHHPWIRRPFFPFHSPSRLFDQFFGEHLLESDLFPTSTSLSPFYLRPPSFLRAPSWIDTGLSEMRLEKDRFSVNLDVKHFSPEELKVKVLGDVIEVHGKHEERQDEHGFISREFHRKYRIPADVDPLTITSSLSSDGVLTVNGPRKQAPGPERTIPITREEKPAVTAAPKK

SEQ ID NO: 24 - protein, 237 aa, Sus scrofa
MRRRLRSEVRPQQSQRDPSSCRRRARLSEYWKLHKTAYIRGWLELQLKELTGQLTLYIHPAAMDIAIHHPWIRRPFFPFHSPSRLFDQFFGEHLLESDLFPASTSLSPFYFRPPSFLRAPSWIDTGLSEMRLEKDRFSVNLDVKHFSPEELKVKVLGDVIEVHGKHEERQDEHGFISREFHRKYRIPADVDPLTITSSLSSDGVLTVNGPRRQASGPERTIPITREEKPAVTAAPKK

SEQ ID NO: 25 - protein, 175 aa, Ovis aries
MDIAIHHPWIRRPFFPFHSPSRLFDQFFGEHLLESDLFPASTSLSPFYLRPPSFLRAPSWIDTGLSEVRLEKDRFSVNLDVKHFSPEELKVKVLGDVIEVHGKHEERQDEHGFISREFHRKYRIPADVDPLTITSSLSSDGVLTMNGPRKQASGPERTIPITREEKPAVTAAPKK

SEQ ID NO: 26 - protein, 173 aa, Homo sapiens
MDVTIQHPWFKRTLGPFYPSRLFDQFFGEGLFEYDLLPFLSSTISPYYRQSLFRTVLDSGISEVRSDRDKFVIFLDVKHFSPEDLTVKVQDDFVEIHGKHNERQDDHGYISREFHRRYRLPSNVDQSALSCSLSADGMLTFCGPKIQTGLDATHAERAIPVSREEKPTSAPSS

SEQ ID NO: 27 - protein, 196 aa, Rattus sp.
MDVTIQHPWFKRALGPFYPSRLFDQFFGEGLFEYDLLPFLSSTISPYYRQSLFRTVLDSGISELMTHMWFVMHQPHAGNPKNNPGKVRSDRDKFVIFLDVKHFSPEDLTVKVLEDFVEIHGKHNERQDDHGYISREFHRRYRLPSNVDQSALSCSLSADGMLTFSGPKVQSGLDAGHSERAIPVSREEKPSSAPSS

SEQ ID NO: 28 - protein, 173 aa, Bos taurus
MDIAIQHPWFKRTLGPFYPSRLFDQFFGEGLFEYDLLPFLSSTISPYYRQSLFRTVLDSGISEVRSDRDKFVIFLDVKHFSPEDLTVKVQEDFVEIHGKHNERQDDHGYISREFHRRYRLPSNVDQSALSCSLSADGMLTFSGPKIPSGVDAGHSERAIPVSREEKPSSAPSS

SEQ ID NO: 29 - protein, 196 aa, Mus sp.
MDVTIQHPWFKRALGPFYPSRLFDQFFGEGLFEYDLLPFLSSTISPYYRQSLFRTVLDSGISELMTHMWFVMHQPHAGNPKNNPVKVRSDRDKFVIFLDVKHFSPEDLTVKVLEDFVEIHGKHNERQDDHGYISREFHRRYRLPSNVDQSALSCSLSADGMLTFSGPKVQSGLDAGHSERAIPVSREEKPSSAPSS

SEQ ID NO: 30 - protein, 173 aa, Gallus gallus
MDITIQHPWFKRALGPLIPSRLFDQFFGEGLLEYDLLPLFSSTISPYYRQSLFRSVLESGISEVRSDRDKFTIMLDVKHFSPEDLSVKIIDDFVEIHGKHSERQDDHGYISREFHRRYRLPANVDQSAITCSLSSDGMLTFSGPKVPSNMDPSHSERPIPVSREEKPTSAPSS

SEQ ID NO: 31 - protein, 173 aa, Canis lupus familiaris
MDIAIQHPWFKRALGPFYPSRLFDQFFGEGLFEYDLLPFLSSTISPYYRQSLFRTVLDSGISEVRSDRDKFVIFLDVKHFSPEDLTVKVLEDFVEIHGKHNERQDDHGYISREFHRRYRLPSNVDQSALSCSLSADGMLTFSGPKVPSGVDAGHSERAIPVSREEKPSSAPSS

SEQ ID NO: 32 - protein, 173 aa, Felis sp.; identical to SEQ ID NO: 31

SEQ ID NO: 33 - protein, 173 aa, Sus scrofa
MDIAIQHPWFKRALGPFYPSRLFDQFFGEGLFEYDLLPFLSSTISPYYRQSLFRTVLDSGVSEVRSDRDKFVIFLDVKHFSPEDLTVKVQEDFVEIHGKHNERQDDHGYISREFHRRYRLPSNVDQSALSCSLSADGMLTFSGPKVPSGVDAGHSERAIPVSREEKPSSAPTS

SEQ ID NO: 34 - protein, 196 aa, Homo sapiens
MADGQMPFSCHYPSRLRRDPFRDSPLSSRLLDDGFGMDPFPDDLTASWPDWALPRLSSAWPGTLRSGMVPRGPTATARFGVPAEGRTPPPFPGEPWKVCVNVHSFKPEELMVKTKDGYVEVSGKHEEKQQEGGIVSKNFTKKIQLPAEVDPVTVFASLSPEGLLIIEAPQVPPYSTFGESSFNNELPQDSQEVTCT

SEQ ID NO: 35 - protein, 142 aa, Escherichia coli
MRNFDLSPLMRQWIGFDKLANALQNAGESQSFPPYNIEKSDDNHYRITLALAGFRQEDLEIQLEGTRLSVKGTPEQPKEEKKWLHQGLMNQPFSLSFTLAENMEVSGATFVNGLLHIDLIRNEPEPIAAQRIAISERPALNS

SEQ ID NO: 36 - protein, 214 aa, Saccharomyces cerevisiae
MSFNSPFFDFFDNINNEVDAFNRLLGEGGLRGYAPRRQLANTPAKDSTGKEVARPNNYAGALYDPRDETLDDWFDNDLSLFPSGFGFPRSVAVPVDILDHDNNYELKVVVPGVKSKKDIDIEYHQNKNQILVSGEIPSTLNEESKDKVKVKESSSGKFKRVITLPDYPGVDADNIKADYANGVLTLTVPKLKPQKDGKNHVKKIEVSSQESWGN

SEQ ID NO: 37 - protein, 170 aa, Homo sapiens
MSHRTSSTFRAERSFHSSSSSSSSSTSSSASRALPAQDPPMEKALSMFSDDFGSFMRPHSEPLAFPARPGGAGNIKTLGDAYEFAVDVRDFSPEDIIVTTSNNHIEVRAEKLAADGTVMNTFAHKCQLPEDVDPTSVTSALREDGSLTIRARRHPHTEHVQQTFRTEIKI

SEQ ID NO: 38 - protein, 162 aa, Rattus sp.
MEIRVPVQPSWLRRASAPLPGFSTPGRLFDQRFGEGLLEAELASLCPAAIAPYYLRAPSVALPTAQVPTDPGYFSVLLDVKHFSPEEISVKVVGDHVEVHARHEERPDEHGFIAREFHRRYRLPPGVDPAAVTSALSPEGVLSIQATPASAQASLPSPPAAK

SEQ ID NO: 39 - protein, 20 aa, Artificial Sequence
  Description of Artificial Sequence Synthetic peptide
DRFSVNLDVKHFSPEELKVK

SEQ ID NO: 40 - protein, 144 aa, Mycobacterium tuberculosis
MATTLPVQRHPRSLFPEFSELFAAFPSFAGLRPTFDTRLMRLEDEMKEGRYEVRAELPGVDPDKDVDIMVRDGQLTIKAERTEQKDFDGRSEFAYGSFVRTVSLPVGADEDDIKATYDKGILTVSVAVSEGKPTEKHIQIRSTN

SEQ ID NO: 41 - protein, 159 aa, Mycobacterium tuberculosis
MNNLALWSRPVWDVEPWDRWLRDFFGPAATTDWYRPVAGDFTPAAEIVKDGDDAVVRLELPGIDVDKDVNVELDPGQPVSRLVIRGEHRDEHTQDAGDKDGRTLREIRYGSFRRSFRLPAHVTSEAIAASYDAGVLTVRVAGAYKAPAETQAQRIAITK

SEQ ID NO: 42 - protein, 102 aa, Homo sapiens
MAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 43 - protein, 196 aa, Homo sapiens; identical to SEQ ID NO: 34

SEQ ID NO: 44 - protein, 160 aa, Strongyloides ratti
MNDRWMTPFVRDPLSVCPLGYGGPANLFNEMNMLERKMMNSLNMVDRNLTNNMELMEPCPEVVNNDKEFRVKMDVSHYGPNELKVTVRDNYLQVEGKHEEKTDKYGTIQRSFVRKYALPKGLTEENVKSELTKDGVLTVGGNKMAIEDKNVKTVPIEYRK

SEQ ID NO: 45 - protein, 180 aa, Loa loa
MSLFRYNPRDYFYTSPMERFIVNLLDNTFDDRSYRPLQSVAPYWLHQPILNECNIGNALGEVLDEKDKFGVQVDVSHFHPKELSVSVRDRELTIEGHHKERTDQSGHGSIERHFVRKYVMPEEVQPDTIESHLSDKGVLTICAAKTTVGTPAARNIPIRASPKEPEAGDKSTSNSTEQSK

SEQ ID NO: 46 - protein, 20 aa, Homo sapiens
DRFSVNLDVKHFSPEELKVK

SEQ ID NO: 47 - protein, 9 aa, Homo sapiens
WIRRPFFPF

SEQ ID NO: 48 - protein, 15 aa, Homo sapiens
EKDRFSVNLDVKHFS

SEQ ID NO: 49 - protein, 20 aa, Homo sapiens
IFLDVKHFSPEDLTVKVQDD

SEQ ID NO: 50 - protein, 15 aa, Homo sapiens
FVIFLDVKHFSPEDL

SEQ ID NO: 51 - protein, 20 aa, Homo sapiens
YSRALSRQLSSGVSEIRHTA

SEQ ID NO: 52 - protein, 15 aa, Homo sapiens
PAYSRALSRQLSSGV